(19) United States
(12) Patent Application Publication
(10) Pub. No.: US 2023/0181710 A1
(43) Pub. Date: Jun. 15, 2023

(54) RECOMBINANT CLASSICAL SWINE FEVER VIRUS

(71) Applicant: Boehringer Ingelheim Vetmedica (China) Co. Ltd., Taizhou, Jiangsu (CN)

(72) Inventors: Ning CHEN, Shanghai (CN); Xiaohong JIANG, Shanghai (CN); Huanhuan LIU, Shanghai (CN); Chao TONG, Shanghai (CN); Jiaying WANG, Shanghai (CN)

(21) Appl. No.: 17/604,298

(22) PCT Filed: Apr. 16, 2020
(86) PCT No.: PCT/CN2020/085036

§ 371 Date: Oct. 15, 2021

(30) Foreign Application Priority Data
Apr. 18, 2019 (WO) PCT/CN2019/083197

(51) Int. Cl. A61K 39/12; C12N 15/86; G01N 33/569; A61P 31/14

(52) U.S. Cl. CPC A61K 39/12; C12N 15/86; G01N 33/56983; A61P 31/14; C12N 2770/24322; C12N 2770/24334; C12N 2770/24343; C12N 2770/24362; C12N 2770/24371; G01N 2469/10; A61K 2039/5254

(57) ABSTRACT

Provided are a recombinant classical swine fever virus comprising at least one substitution within the epitope of the E2 protein specifically recognized by the 6B8 monoclonal antibody, an immunogenic composition comprising said CSFV, the use of said immunogenic composition for preventing and/or treating diseases associated with CSFV in an animal, a method or a kit for differentiating animals infected with CSFV from animals vaccinated with said immunogenic composition, and an attenuated classical swine fever viruses.

### CROSS-REFERENCE TO RELATED APPLICATIONS

This Application is a national stage application under 35 U.S.C. § 371 of International Patent Application No. PCT/CN2020/085036, filed Apr. 16, 2020, which claims the benefit of and priority to International Patent Application No. PCT/CN2019/083197, filed Apr. 18, 2019, the entire contents of which are hereby expressly incorporated by reference herein.

### INCORPORATION BY REFERENCE OF SEQUENCE LISTING

The content of the text file named “WP202101206_seq.TXT”, which was created on Apr. 18, 2019 and is 327,680 bytes in size, is hereby incorporated by reference in its entirety.

### TECHNICAL FIELD

The present invention relates the field of animal health. Particularly, the present invention relates to a recombinant classical swine fever virus comprising at least one mutation within the 6B8 epitope of the E2 protein, wherein the (unmodified) 6B8 epitope is specifically recognized by the 6B8 monoclonal antibody. Further, the present invention provides an immunogenic composition comprising the recombinant CSFV of the present invention and the use of the immunogenic composition for preventing and/or treating diseases associated with CSFV in an animal. Moreover, the present invention provides a method and a kit for differentiating animals infected with CSFV from animals vaccinated with the immunogenic composition of the present invention.

### TECHNICAL BACKGROUND

Classical swine fever (CSF) is a highly contagious disease of pigs and wild boars that causes significant economic losses. The causative agent of the disease is classical swine fever virus (CSFV). In China, a combination of prophylactic vaccination and stamping out strategy is implemented to control CSF outbreaks. However, sporadic CSF outbreaks and persistent infection are still reported in most parts of China.

The currently available CSFV vaccines are based on C-strain that has been attenuated by serial passaging in rabbit and has been proven to be safe and efficacious against CSF. A strong immune response is elicited within 5 days post vaccination that provides solid protection. However, one of the limitations is that animals infected by field strains cannot be differentiated from those vaccinated by standard serological means.

There is still a need in the art for a new CSFV vaccine that is safe, effective and animals vaccinated by which can be differentiated from those infected by wild type field strains.

### BRIEF DESCRIPTION OF THE INVENTION

In one aspect, the present invention provides a recombinant CSFV (classical swine fever virus) comprising at least one mutation within the 6B8 epitope of the E2 protein, wherein the (unmodified) 6B8 epitope is specifically recognized by the 6B8 monoclonal antibody, and wherein the 6B8 monoclonal antibody is produced by a hybridoma deposited at CCTCC under the accession number CCTCC C2018120, or wherein the 6B8 monoclonal antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or wherein the 6B8 monoclonal antibody comprises the CDRs of the monoclonal antibody produced by a hybridoma deposited at CCTCC under the accession number CCTCC C2018120, or wherein the 6B8 monoclonal antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

In one aspect, the present invention provides an attenuated CSFV, which may or may not comprise one or more mutations within the 6B8 epitope of the E2 protein as disclosed herein. In one aspect, the present invention provides attenuated CSFV, which have at least one mutation within the Erns protein and/or a mutation within the Npro protein that causes attenuation. Preferably, the mutation within the Erns protein is a deletion of amino acid position 79 and/or a deletion of amino acid position 171, and the mutation within the Npro is a deletion of the Npro protein except for the first four amino terminal amino acids. In one aspect, the present invention provides an attenuated CSFV, which have at least one mutation within the Erns protein and/or a mutation within the Npro protein that causes attenuation as disclosed herein, and a mutation within the 6B8 epitope of the E2 protein as provided herein. In one aspect, the attenuated CSFV according to the invention, which may or may not comprise one or more mutations within the 6B8 epitope of the E2 protein as disclosed herein, is derived from C-strain or field strain QZ07 or GD18.

In one aspect, the present invention provides an isolate nucleic acid coding for a recombinant CSFV of the present invention.

In one aspect, the present invention provides a vector comprising the nucleic acid of the present invention.

In one aspect, the present invention provides an immunogenic composition comprising the recombinant CSFV according to the present invention.

In one aspect, the present invention provides a method of preventing and/or treating diseases associated with CSFV in an animal, the method comprising the step of administering the immunogenic composition of the present invention to an animal in need thereof.

In one aspect, the present invention provides a method of marking a CSFV vaccine comprising introducing into a CSFV at least one mutation within the 6B8 epitope of the E2 protein, wherein the (unmodified) 6B8 epitope is specifically recognized by the 6B8 monoclonal antibody, and wherein the 6B8 monoclonal antibody is produced by a hybridoma deposited at CCTCC under the accession number CCTCC C2018120, or wherein the 6B8 monoclonal antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or wherein the 6B8 monoclonal antibody comprises the CDRs of the monoclonal antibody produced by a hybridoma deposited at CCTCC under the accession number CCTCC C2018120, or wherein the 6B8 monoclonal antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

In one aspect, the present invention provides a method of differentiating animals infected with CSFVfrom animals vaccinated with the immunogenic composition of the present invention, comprising a) obtaining a sample from an animal; and b) analyzing said sample in an immuno test.

In one aspect, the present invention provides an antibody or an antigen-binding fragment thereof, wherein said antibody is produced by a hybridoma deposited at CCTCC under the accession number CCTCC C2018120, or wherein said antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or wherein the antibody comprises the CDRs of the monoclonal antibody produced by a hybridoma deposited at CCTCC under the accession number CCTCC C2018120, or wherein the antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

In one aspect, the present invention provides a kit for differentiating animals infected with CSFV from animals vaccinated with the immunogenic composition of the present invention, which comprises the antibody of the present invention, or an antigen-binding fragment thereof.

### BRIEF DESCRIPTION OF THE DRAWINGS

FIG. 1: Growth characteristics of attenuation candidate viruses with double deletions or single deletion.

FIG. 2: mAb 6B8 recognizes most CSFV strains while has no reaction with BVDV viruses.

FIG. 3: Sequence alignment of CSFV isolates, BVDV strains and some other Pestiviruses.

FIG. 4: IFA results showing that amino acid residues at position 14, 22, or 24/25 are critical for mAb 6B8 binding.

FIG. 5: IFA results of rescued viruses of GD18-ddNpro-ErnsH with various DIVA mutations.

FIG. 6: Growth curve of GD18-ddNpro-ErnsH P10 viruses with various DIVA mutations. Different 6B8 epitope mutations yielded viable GD18 candidates in vitro and showed growth ability comparable to original attenuated GD18-ddNpro-ErnsH candidates.

FIG. 7: IFA results of rescued viruses of various attenuation candidates with DIVA mutation KARD. Candidates containing four amino acids mutation KARD were infectious in vitro and did not react with 6B8 mAb.

FIG. 8: Body temperature (Mean) in safety experiments.

FIG. 9: Body temperature (Mean) in efficacy experiments.

FIG. 10: Total clinical score (Mean) in efficacy experiments.

FIG. 11: 6B8 dcELISA results showing that different mutant forms of 6B8 epitope, RD, KRD, and KARD are all feasible as DIVA option.

FIG. 12: Comparison of cELISA and dcELISA for DIVA. Differentiation of overdose vaccination sera and challenge sera can be achieved by DIVA dcELISA.

### DETAILED DESCRIPTION

Before the aspects of the present invention are described, it must be noted that as used herein and in the appended claims, the singular forms “a”, “an”, and “the” include plural reference unless the context clearly dictates otherwise. Thus, for example, reference to “a or an epitope” includes a plurality of epitopes, reference to the “virus” is a reference to one or more viruses and equivalents thereof known to those skilled in the art, and so forth. The term “and/or” is intended to encompass any combinations of the items connected by this term, equivalent to listing all the combinations individually. For example, “A, B and/or C” encompasses “A”, “B”, “C”, “A and B”, “A and C”, “B and C”, and “A and B and C”. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the virus strains, the cell lines, vectors, and methodologies as reported in the publications which might be used in connection with the invention. Nothing herein is to be construed as an admission that the invention is not entitled to antedate such disclosure by virtue of prior invention.

In one aspect, the present invention provides a recombinant CSFV (classical swine fever virus) comprising at least one mutation within the 6B8 epitope of the E2 protein, wherein the (unmodified) 6B8 epitope is specifically recognized by the 6B8 monoclonal antibody. In one aspect, the recombinant CSFV of the invention is derived from a wildtype CSFV having a 6B8 epitope specifically recognized by the 6B8 monoclonal antibody in its E2 protein. In one aspect, the 6B8 monoclonal antibody is produced by a hybridoma deposited at CCTCC under the accession number CCTCC C2018120, or the 6B8 monoclonal antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or the 6B8 monoclonal antibody comprises the CDRs of the monoclonal antibody produced by a hybridoma deposited at CCTCC under the accession number CCTCC C2018120, or the 6B8 monoclonal antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

The term “CSFV” as used herein refers to all viruses belonging to species of classical swine fever virus (CSFV) in the genus Pestivirus within the family Flaviviridae.

The term “recombinant” refers to a CSFV that has been altered, rearranged, or modified by genetic engineering. However, the term does not refer to alterations in polynucleotide or amino acid sequence that result from naturally occurring events, such as spontaneous mutations.

“The 6B8 epitope of the E2 protein” herein also refers to an epitope of the E2 protein specifically recognized by the 6B8 monoclonal antibody as defined herein. The 6B8 epitope may comprise at least the amino acid sequence STNEIGPLGAEG (SEQ ID NO:11) or STDEIGLLGAGG (SEQ ID NO:12).

The term “6B8 monoclonal antibody” refers to the 6B8 monoclonal antibody or an antigen-binding fragment thereof, wherein the 6B8 monoclonal antibody specifically recognizes the 6B8 epitope, in particular the 6B8 epitope that comprises at least the amino acid sequence STNEIGPLGAEG (SEQ ID NO:11) or STDEIGLLGAGG (SEQ ID NO:12). Preferably, the term 6B8 monoclonal antibody refers to a monoclonal antibody that comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30. More preferably, the term 6B8 monoclonal antibody refers to a monoclonal antibody that comprises a heavy chain variable region (V_{H}) having an amino acid sequence as set forth in SEQ ID NO: 9 and a light chain variable region (V_{L}) having an amino acid sequence as set forth in SEQ ID NO: 10. More preferably the term 6B8 monoclonal antibody refers to the monoclonal antibody produced by a hybridoma deposited at CCTCC under the accession number CCTCC C2018120.

The term “antigen-binding fragment of the 6B8 monoclonal antibody” refers to a fragment of the 6B8 monoclonal antibody or at least encodes for an amino acid sequence that specifically recognizes the 6B8 epitope, in particular the 6B8 epitope that comprises at least the amino acid sequence STNEIGPLGAEG (SEQ ID NO:11) or STDEIGLLGAGG (SEQ ID NO:12). The term further encompasses an amino acid fragment coding for a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, and/or a VL CDR1 comprising the amino acid sequence set forth in SEQ ID NO:28, a VL CDR2 comprising the amino acid sequence set forth in SEQ ID NO:29, and a VLCDR3 comprising the amino acid sequence set forth in SEQ ID NO:30. Moreover, the term also encompasses an amino acid fragment that comprises a heavy chain variable region (V_{H}) having an amino acid sequence as set forth in SEQ ID NO: 9 and/or a light chain variable region (V_{L}) having an amino acid sequence as set forth in SEQ ID NO: 10. More preferably the term encompasses an amino acid fragment encoded by the monoclonal antibody produced by a hybridoma deposited at CCTCC under the accession number CCTCC C2018120, which amino acid fragment specifically binds to the 6B8 epitope.

The term “mutation” includes substitution, deletion or addition of one or more amino acids. The term mutation is well known to the person skilled in the art and the person skilled in the art can generate mutations without further ado.

In one aspect, the at least one mutation within the 6B8 epitope of the E2 protein of the invention leads to a specific inhibition of the binding of the 6B8 monoclonal antibody to such mutated 6B8 epitope.

The term “specifically inhibits” or “specific inhibition” means that the 6B8 antibody binds with an at least 2-times, preferably 5-times, more preferably 10-times and even more preferably 50-times lower affinity to the mutated 6B8 epitope in comparison to the unmodified 6B8 epitope, in particular to the unmodified 6B8 epitope having the amino acid sequence STNEIGPLGAEG (SEQ ID NO: 1) or STDEIGLLGAGG (SEQ ID NO: 2). “Affinity” is the interaction between a single antigen-binding site on an antibody molecule and a single epitope. It is expressed by the association constant KA=kass/kdiss, or the dissociation constant KD=kdiss/kass. More preferably, the term “specifically inhibits” or “specific inhibition” means that the 6B8 monoclonal antibody as defined herein, in particular the monoclonal antibody produced by a hybridoma deposited at CCTCC under the accession number CCTCC C2018120 does not detectably bind to the mutated 6B8 epitope according the invention in an specific immunofluorescence assay, preferably in the specific immunofluorescence assay as described in example 6.

The term “substitution” means that an amino acid is replaced by another amino acid at the same position. Thus, the term “substitution” covers the removal/deletion of an amino acid, followed by insertion of another amino acid at the same position.

The term “E2 protein” refers to the processed E2 protein which results as final cleavage product from the polyprotein (Npro-C-Erns-E1-E2-p7-NS2-NS3-NS4A-NS4B-NS5A-NS5B) of the CSFV. For example, the E2 protein of the field strain QZ07 has the amino acid sequence set forth in SEQ ID NO:7, the E2 protein of the field strain GD18 has the amino acid sequence set forth in SEQ ID NO:8, the E2 protein of the C-strain has the amino acid sequence set forth in SEQ ID NO:35.

In one aspect of the invention, the 6B8 epitope of the E2 protein specifically recognized by the 6B8 monoclonal antibody is defined at least by the amino acid residue at position 14, position 22, position 24 and/or positions 24 and 25 (“24/25”) of the E2 protein.

In one aspect of the invention, the 6B8 epitope of the E2 protein specifically recognized by the 6B8 monoclonal antibody is defined at least by the amino acid residue S14, G22, E24, and/or E24/G25 of the E2 protein, such as for isolates QZ07, GD18 or GD191. In one aspect of the invention, the 6B8 epitope of the E2 protein specifically recognized by the 6B8 monoclonal antibody is defined at least by the amino acid residue S14, G22, G24, and/or G24/G25 of the E2 protein, such as for C-strain.

The numbering of the amino acid residue refers to the amino acid position in the processed E2 protein from the N-terminal, e.g. to the amino acid position as provide in SEQ ID NO:7 in an exemplary manner. However, the amino acid position can further be defined in relation to the polyprotein (containing Npro-C-Erns-E1-E2-p7-NS2-NS3-NS4A-NS4B-NS5A-NS5B).

In one aspect of the invention, the 6B8 epitope of the E2 protein specifically recognized by the 6B8 monoclonal antibody is defined at least by the amino acid sequence STN EIGPLGAEG (SEQ ID NO:11) (such as for isolates QZ07, GD18 or GD191) or STDEIGLLGAGG (such as for C-strain). In one aspect of the invention, the 6B8 epitope of the E2 protein specifically recognized by the 6B8 monoclonal antibody is defined at least by the amino acid sequence STNEIGPLGAEG (SEQ ID NO:11) (such as for isolates QZ07, GD18 or GD191). In one aspect of the invention, the 6B8 epitope of the E2 protein specifically recognized by the 6B8 monoclonal antibody is defined at least by the amino acid sequence STDEIGLLGAGG (SEQ ID NO:12) (such as for C-strain).

In one aspect of the invention, the recombinant CSFV according to the invention comprises a substitution at amino acid position 24 of the E2 protein, a substitution at amino acid positions 24/25 of the E2 protein, a substitution at amino acid position 14 of the E2 protein, and/or a substitution at amino acid position 22 of the E2 protein.

In one aspect of the invention, the recombinant CSFV according to the invention comprises a substitution at amino acid position 24 of the E2 protein and a substitution at amino acid position 25 of the E2 protein.

In one aspect of the invention, the recombinant CSFV according to the invention comprises a substitution at amino acid position 24 of the E2 protein and a substitution at amino acid position 14 of the E2 protein.

In one aspect of the invention, the recombinant CSFV according to the invention comprises a substitution at amino acid position 24 of the E2 protein, a substitution at amino acid position 25 of the E2 protein and a substitution at amino acid position 14 of the E2 protein.

In one aspect of the invention, the recombinant CSFV according to the invention comprises a substitution at amino acid position 24 of the E2 protein and a substitution at amino acid position 22 of the E2 protein.

In one aspect of the invention, the recombinant CSFV according to the invention comprises a substitution at amino acid position 24 of the E2 protein, a substitution at amino acid position 25 of the E2 protein and a substitution at amino acid position 22 of the E2 protein.

In one aspect of the invention, the recombinant CSFV according to the invention comprises a substitution at amino acid position 14 of the E2 protein and a substitution at amino acid position 22 of the E2 protein.

In one aspect of the invention, the recombinant CSFV according to the invention comprises a substitution at amino acid position 24 of the E2 protein, a substitution at amino acid position 14 of the E2 protein, and a substitution at amino acid position 22 of the E2 protein.

In one aspect of the invention, the recombinant CSFV according to the invention comprises a substitution at amino acid position 24 of the E2 protein, a substitution at amino acid position 25 of the E2 protein, a substitution at amino acid position 14 of the E2 protein, and a substitution at amino acid position 22 of the E2 protein.

In one aspect of the invention, in the recombinant CSFV according to the invention, the amino acid at position 24 of the E2 protein is substituted to R or K, the amino acid at position 24 is substituted to R or K and the amino acid at position 25 of the E2 protein is substituted to D respectively, the amino acid at position 14 of the E2 protein is substituted to K, Q or R, and/or the amino acid at position 22 of the E2 protein is substituted to A, R, Q, or E, with A and R being preferred.

In one aspect of the invention, in the recombinant CSFV according to the invention, the amino acid at position 24 of the E2 protein is substituted to R or K, and the amino acid at position 25 of the E2 protein is substituted to D.

In one aspect of the invention, in the recombinant CSFV according to the invention, the amino acid at position 24 of the E2 protein is substituted to R or K, and the amino acid at position 14 of the E2 protein is substituted to K, Q or R.

In one aspect of the invention, in the recombinant CSFV according to the invention, the amino acid at position 24 of the E2 protein is substituted to R or K, the amino acid at position 25 of the E2 protein is substituted to D, and the amino acid at position 14 of the E2 protein is substituted to K, Q or R.

In one aspect of the invention, in the recombinant CSFV according to the invention, the amino acid at position 24 of the E2 protein is substituted to R or K, and the amino acid at position 22 of the E2 protein is substituted to A, R, Q, or E, with A and R being preferred.

In one aspect of the invention, in the recombinant CSFV according to the invention, the amino acid at position 24 of the E2 protein is substituted to R or K, the amino acid at position 25 of the E2 protein is substituted to D, and the amino acid at position 22 of the E2 protein is substituted to A, R, Q, or E, with A and R being preferred.

In one aspect of the invention, in the recombinant CSFV according to the invention, the amino acid at position 24 of the E2 protein is substituted to R or K, the amino acid at position 14 of the E2 protein is substituted to K, Q or R, and the amino acid at position 22 of the E2 protein is substituted to A, R, Q, or E, with A and R being preferred.

In one aspect of the invention, in the recombinant CSFV according to the invention, the amino acid at position 24 of the E2 protein is substituted to R or K, the amino acid at position 25 of the E2 protein is substituted to D, the amino acid at position 14 of the E2 protein is substituted to K, Q or R, and the amino acid at position 22 of the E2 protein is substituted to A, R, Q, or E, with A and R being preferred.

In one aspect of the invention, the recombinant CSFV according to the invention comprises a substitution of E or G to R or K at amino acid position 24 of the E2 protein, a substitution of E or G to R or K at amino acid position 24 of the E2 protein and a substitution of G to D at amino acid position 25 of the E2 protein, a substitution of S to K, Q or R at amino acid position 14 of the E2 protein, and/or a substitution of G to A, R, Q, or E, with A and R being preferred at amino acid position 22 of the E2 protein.

In one aspect of the invention, the recombinant CSFV according to the invention comprises a substitution of E or G to R or K at amino acid position 24 of the E2 protein and a substitution of G to D at amino acid position 25 of the E2 protein.

In one aspect of the invention, the recombinant CSFV according to the invention comprises a substitution of E or G to R or K at amino acid position 24 of the E2 protein, and a substitution of S to K, Q or R at amino acid position 14 of the E2 protein.

In one aspect of the invention, the recombinant CSFV according to the invention comprises a substitution of E or G to R or K at amino acid position 24 of the E2 protein, a substitution of G to D at amino acid position 25 of the E2 protein, and a substitution of S to K, Q or R at amino acid position 14 of the E2 protein.

In one aspect of the invention, the recombinant CSFV according to the invention comprises a substitution of E or G to R or K at amino acid position 24 of the E2 protein, a substitution of S to K, Q or R at amino acid position 14 of the E2 protein, and a substitution of G to A, R, Q, or E, with A and R being preferred, at amino acid position 22 of the E2 protein.

In one aspect of the invention, the recombinant CSFV according to the invention comprises a substitution of E or G to R or K at amino acid position 24 of the E2 protein, a substitution of G to D at amino acid position 25 of the E2 protein, a substitution of S to K, Q or R at amino acid position 14 of the E2 protein, and a substitution of G to A, R, Q, or E, with A and R being preferred, at amino acid position 22 of the E2 protein.

In one aspect of the invention, the amino acid substitution within the 6B8 epitope of the E2 protein of the recombinant CSFV according to the invention results in a mutated 6B8 epitope sequence KTNEIGPLGARD (SEQ ID NO:13) or KTNEIGPLAARD (SEQ ID NO:14) or STNEIGPLGARD (SEQ ID NO:31) or STDEIGLLGARD (SEQ ID NO:32) or KTDEIGLLGARD (SEQ ID NO:33) or KTDEIGLLAARD (SEQ ID NO:34). In one aspect of the invention, the amino acid substitution within the 6B8 epitope of the E2 protein results in a mutated 6B8 epitope sequence KTNEIGPLGARD (SEQ ID NO:13). In one aspect of the invention, the amino acid substitution within the 6B8 epitope of the E2 protein results in a mutated 6B8 epitope sequence KTNEIGPLAARD (SEQ ID NO:14). In one aspect of the invention, the amino acid substitution within the 6B8 epitope of the E2 protein results in a mutated 6B8 epitope sequence STNEIGPLGARD (SEQ ID NO:31). In one aspect of the invention, the amino acid substitution within the 6B8 epitope of the E2 protein results in a mutated 6B8 epitope sequence STDEIGLLGARD (SEQ ID NO:32). In one aspect of the invention, the amino acid substitution within the 6B8 epitope of the E2 protein results in a mutated 6B8 epitope sequence KTDEIGLLGARD (SEQ ID NO:33). In one aspect of the invention, the amino acid substitution within the 6B8 epitope of the E2 protein results in a mutated 6B8 epitope sequence KTDEIGLLAARD (SEQ ID NO:34).

In one aspect of the invention, the E2 protein of the recombinant CSFV of the invention comprises an amino acid sequence having at least 75%, at least 80%, at least 85%, at least 90%, at least 91%, at least 92%, at least 93%, at least 94%, at least 95%, at least 96%, at least 97%, at least 98%, at least 99% sequence identity to any one of SEQ ID NO:7, 8 and 35, but contains the mutations within the 6B8 epitope as defined herein above.

In a preferred aspect of the invention, the E2 protein having at least one mutation within the 6B8 epitope as disclosed herein is immunogenic and preferably confers protective immunity against CSFV. The E2 protein contains four antigenic domains, A, B, C and D domain, and all these domains are located at the N-terminal of the E2 protein. The four domains constitute two independent antigenic units, one is the unit of B/C domains and the other comprises A/D domain. The B/C domain is from amino acid position 1 to positions 84/111 and D/A domain is located from amino acid position 77 to positions 111/177. Furthermore, the B/C domain is linked by a putative disulfide bond between amino acid 4C and 48C, while the unit D/A is formed with two disulfide bonds, one between amino acids 103C and 167C, and the other between amino acids 129C and 139C. Those Cysteine residues are crucial for conformation antigenic structure of E2 protein. Antigenic motif (82-85LLFD) are important for the antigenic structure of E2 protein for convalescent serum binding. Another motif (RYLASLHKKALPT, amino acid positions 64 to 76) is also identified important for the structural integrity of conformational epitope recognition of E2 protein. In addition it is reported that E2 protein containing merely one of above mentioned antigenic domain remained immunogenic and can protects pigs from infectious CSFV challenge. Therefore, in a preferred aspect of the invention, the E2 protein having at least one modification within the 6B8 epitope as described herein retains at least one, preferably at least one of the antigenic domains as described above. Preferably, the E2 protein of the invention can confer protective immunity against CSFV. In one aspect, the at least one mutation within the 6B8 epitope as defined herein can be introduced without substantially affects the protective immunogenicity of the E2 protein against CSFV.

“Sequence identity” between two polypeptide sequences indicates the percentage of amino acids that are identical between the sequences. Methods for evaluating the level of sequence identity between amino acid or nucleotide sequences are known in the art. For example, sequence analysis softwares are often used to determine the identity of amino acid sequences. For example, identity can be determined by using the BLAST program at NCBI database. For determination of sequence identity, see e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987 and Sequence Analysis Primer, Gribskov, M. and Devereux, J., eds., M Stockton Press, New York, 1991.

In one aspect of the invention, the amino acid substitution within the 6B8 epitope of the E2 protein is a stable amino acid substitution. Said stable amino acid substitution results in a stable recombinant CSFV according to the invention.

The term “stable amino acid substitution” refers to an amino acid substitution which is still present after several passages of the CSFV virus in cell culture. Preferably, the amino acid substitution within the 6B8epitope of the E2 protein is still present after at least 3 passages, more preferably after at least 6 passages, even more preferably after at least 9 passages, even more preferably after at least 12 passages, even more preferably after at least 15 passages, even more preferably after at least 20 passages, even more preferably after at least 30 passages, even more preferably after at least 50 passages, even more preferably after 100 passages, most preferred after 250 passages of the CSFV in cell culture. The term “cell culture” or “passages in cell culture” is known by the person skilled in the art. The term relates to the propagation of the virus in cells cultured outside the organism. Said term also refers to the propagation of cells outside the organism in a cell system. Such cell system comprises host cells (such as SK-6 cells, ST cells or PK-15 cells and the alike) and cell culture medium suitable for the propagation of such cells outside of the organism. Suitable cell culture media are known to a person skilled in the art and are commercially available. They may comprise nutrients, salts, growth factors, antibiotics, serum (e.g. fetal calf serum) and pH-indicators (e.g. phenol red). Whether an amino acid is still present within the 6B8 epitope of the E2 protein can be determined by the person skilled in the art without further ado. Further, the term “stable amino acid substitution” also refers to an amino acid substitution which is still present after re-isolation of the CSFV from vaccinated animals which prior have been vaccinated with the CSFV of the present invention. Preferably, the amino acid substitution within the 6B8 epitope of the E2 protein is still present at least 3 days, more preferably at least 4 days, even more preferably at least 5 days, even more preferably at least 6 days, even more preferably at least 7 days, even more preferably at least 8 days, even more preferably at least 9 days, even more preferably at least 10 days, even more preferably at least 12 days, even more preferably at least 15 days, even more preferably at least 20 days, even more preferably at least 25 days, even more preferably at least 35 days, even more preferably at least 50 days, most preferred at least 100 days after the vaccination in the re-isolated CSFV from vaccinated animals which prior have been vaccinated with the CSFV of the present invention. The vaccination, re-isolation of the CSFV and the determination whether an amino acid is still present within the 6B8 epitope of the E2 protein can be done by the person skilled in the art.

Surprisingly, it has been found that the substitutions within the 6B8 epitope of the E2 protein is highly suitable for generating marker or DIVA vaccines due to the stability of said substitutions. Said substitution within the 6B8 epitope according to the present invention is stable after several passages of the CSFV virus according to the invention in cell culture. Moreover, it has been shown that the substitutions within the 6B8 epitope according to the present invention cannot be recognized by antibodies specific for the intact (wildtype) 6B8 epitope of the E2 protein. Thus, the substitution within the 6B8 epitope according to the present invention can be used as a negative marker for generating marker or DIVA vaccines. In one aspect, said at least one substitution within the 6B8 epitope of the E2 protein specifically recognized by the 6B8 monoclonal antibody inhibits the binding of 6B8 to the E2 protein.

In one aspect of the invention, the recombinant CSFV is attenuated.

The term “attenuated” means that the virulence of the CSFV has been reduced. In the present invention “attenuation” is synonymous with “avirulent”. In the present invention, an attenuated CSFV is one in which the virulence has been reduced so that it does not cause clinical signs of a CSFV infection but is capable of inducing an immune response in the target animal, but may also mean that the clinical signs are reduced in incidence or severity in animals infected with the attenuated CSFV in comparison with a “control group” of animals infected with non-attenuated CSFV and not receiving the attenuated virus. In this context, the term “reduce/reduced” means a reduction of at least 10%, preferably 25%, even more preferably 50%, most preferably of more than 100% as compared to the control group as defined above. Thus, an attenuated CSFV strain is one that suitable for incorporation into an immunogenic composition.

The attenuation of the CSFV can be done by serial passaging. The attenuation by serial passaging of the CSFV in cell culture is well known by the person skilled in the art and can be done by the person skilled in the art without further ado.

Further, attenuation can be achieved by mutating the CSFV. Attenuated CSFV strains can be generated by mutation of the Erns gene (WO 99/64604, WO2005/111201, WO 2009/156448 A1, Mayer et a I., 2003. Virus Res. 98: 105-16, Meyers et al., 1999. J. Virol. 73: 10224-10235, Widjojoatmodjo et al., 2000. J. Virol. 74: 2973-80), by deletion of Npro from CSFV virulent strains (Tratschin, J., et al., 1998. J. Virol. 72: 7681-7684), by combining mutations in Erns and deletion of Npro (WO2005/111201, WO 2009/156448 A1), by combining mutations in Erns and E2 (van Gen nip et al. 2004. J. Virol, 78: 3812-3823), by mutation of the E1 gene (Risatti et al., 2005. Virology 343: 116-127), and by mutation of the E2 gene (Risatti et al., 2007. Virology 364: 371-82).

In one aspect of the invention, the recombinant CSFV has at least one mutation in the Erns protein and/or at least one mutation in the Npro protein. In one aspect, said at least one mutation in the Erns protein and/or at least one mutation in the Npro protein results in attenuation of the recombinant CSFV. Mutations within the sequence of Npro and Erns already have been described in the prior art as set forth above (see exemplary WO 99/64604, WO2005/111201 A, WO2009/156448 A1).

In one aspect of the invention, the mutation in the Erns protein is a deletion of amino acid at amino acid position 79 of Erns protein and/or a deletion of amino acid at amino acid position 171 of Erns protein. In one aspect of the invention, the mutation in Npro protein is a deletion of the Npro protein except for the first four amino terminal amino acids. The amino acid position refers to the position in the processed Erns protein (e.g. to the amino acid position as provide in SEQ ID NO:16 in an exemplary manner) or processed Npro protein (e.g. to the amino acid position as provide in SEQ ID NO:15 in an exemplary manner), respectively.

In one aspect of the invention, the recombinant CSFV has a deletion of amino acid at amino acid position 79 of Erns protein. In one aspect of the invention, the recombinant CSFV has a deletion of amino acid at amino acid position 79 of Erns protein and a deletion of amino acid at amino acid position 171 of Erns protein. In one aspect of the invention, the recombinant CSFV has a deletion of amino acid at amino acid position 79 of Erns protein and a deletion of the Npro protein except for the first four amino terminal amino acids. In one aspect of the invention, the recombinant CSFV has a deletion of amino acid at amino acid position 79 of Erns protein, a deletion of amino acid at amino acid position 171 of Erns protein, and a deletion of the Npro protein except for the first four amino terminal amino acids.

The term “Npro” as understood herein relates to the first protein encoded by the viral open reading frame and cleaves itself from the rest of the synthesized polyprotein (Stark, et al., J. Virol. 67:7088-7093 (1993); Wiskerchen, et al., Virol. 65:4508-4514 (1991)). Said term, depending on the context, may also relate to the remaining “Npro” amino acids after mutation of the sequence for said protein itself. For example, the amino acid sequence of the Npro protein of the field strain GD18 is set forth in SEQ ID NO:15, the amino acid sequence of the Npro protein of the field strain QZ07 is set forth in SEQ ID NO:18.

“Erns” as used herein relates to the glycoprotein Erns which represents a structural component of the pestivirus virion (Thiel et al., 1991. J. Virol. 65: 4705-4712). Erns lacks a typical membrane anchor and is secreted in considerable amounts from the infected cells; this protein has been reported to exhibit RNase activity (Hulst et al., 1994. Virology 200: 558-565; Schneider et al., 1993. Science 261: 1169-1171; Windisch et al., 1996. J. Virol. 70: 352-358). It should be noted that the term glycoprotein E0 is often used synonymously to glycoprotein Erns in publications. Said term, depending on the context, may also relate to the mutated “Erns” protein. For example, the amino acid sequence of the Erns protein of the field strain GD18 is set forth in SEQ ID NO:16, the amino acid sequence of the Erns protein of the field strain QZ07 is set forth in SEQ ID NO:19.

The term “deletion of Npro protein except for the first four amino terminal amino acids” as used herein refers to the deletion of almost the complete Npro coding region; however, four amino terminal amino acids remain.

A person skilled in the art would acknowledge that the recombinant CSFV of the invention can be derived from various CSFV isolates, as the 6B8 epitope is evolutionarily conserved among different CSFV strains.

In one aspect of the invention, the recombinant CSFV of the invention is derived from an isolate of genogroup 2.1. In one aspect of the invention, the recombinant CSFV is derived for example from the field strain GD18 or QZ07. The field strain QZ07 has a full length nucleotide sequence as shown in SEQ ID NO: 1, or comprises or expresses a polyprotein with the amino acid sequence set forth in SEQ ID NO:20. The field strain GD18 has a full length nucleotide sequence as shown in SEQ ID NO: 2, or comprises or expresses a polyprotein with the amino acid sequence set forth in SEQ ID NO:17.

In one aspect of the invention, the recombinant CSFV of the invention is derived from an isolate of genogroup 1. In one aspect of the invention, the recombinant CSFV is derived from the C-strain well known in the art.

In one aspect, the invention provides a recombinant CSFV comprising a deletion of amino acid at amino acid position 79 of Erns protein and at least a mutation within the 6B8 epitope of the E2 protein as disclosed herein.

In one aspect of the invention, the recombinant CSFV is derived, for example from a field strain QZ07, and comprises a deletion of amino acid at amino acid position 79 of Erns protein, a substitution of E to R or K at amino acid position 24 of the E2 protein, or a substitution of E to R or K at amino acid position 24 of the E2 protein and a substitution of G to D at amino acid position 25 of the E2 protein, and optionally further comprises a substitution of S to K, Q or R at amino acid position 14 of the E2 protein and/or a substitution of G to A, R, Q, or E, with A and R being preferred, at amino acid position 22 of the E2 protein.

In one aspect, the recombinant CSFV has a full length nucleotide sequence as shown in SEQ ID NO: 3, or comprises or expresses a polyprotein with the amino acid sequence set forth in SEQ ID NO:21.

In one aspect, the present invention provides a recombinant CSFV comprising a deletion of amino acid at amino acid position 79 of Erns protein and a deletion of amino acid at amino acid position 171 of Erns protein and at least a mutation within the 6B8 epitope of the E2 protein as disclosed herein.

In one aspect of the invention, the recombinant CSFV is derived, for example from a field strain QZ07, and comprises a deletion of amino acid at amino acid position 79 of Erns protein, a deletion of amino acid at amino acid position 171 of Erns protein, a substitution of E to R or K at amino acid position 24 of the E2 protein, or a substitution of E to R or K at amino acid position 24 of the E2 protein and a substitution of G to D at amino acid position 25 of the E2 protein, and optionally further comprises a substitution of S to K, Q or R at amino acid position 14 of the E2 protein and/or a substitution of G to A, R, Q, or E, with A and R being preferred, at amino acid position 22 of the E2 protein.

In one aspect, the recombinant CSFV has a full length nucleotide sequence as shown in SEQ ID NO: 4, or comprises or expresses a polyprotein with the amino acid sequence set forth in SEQ ID NO:22.

In one aspect, the present invention provides a recombinant CSFV comprising a deletion of amino acid at amino acid position 79 of Erns protein and a deletion of the Npro protein except for the first four amino terminal amino acids and at least another mutation within the 6B8 epitope of the E2 protein as disclosed herein.

In one aspect of the invention, the recombinant CSFV is derived, for example from a field strain GD18, and comprises a deletion of amino acid at amino acid position 79 of Erns protein, a deletion of the Npro protein except for the first four amino terminal amino acids, a substitution of E to R or K at amino acid position 24 of the E2 protein, or a substitution of E to R or K at amino acid position 24 of the E2 protein and a substitution of G to D at amino acid position 25 of the E2 protein, and optionally further comprises a substitution of S to K, Q or R at amino acid position 14 of the E2 protein and/or a substitution of G to A, R, Q, or E, with A and R being preferred, at amino acid position 22 of the E2 protein.

In one aspect, the recombinant CSFV has a full length nucleotide sequence as shown in SEQ ID NO: 5 or comprises or expresses a polyprotein with the amino acid sequence set forth in SEQ ID NO:23.

In one aspect of the invention, the recombinant CSFV is derived, for example from a field strain GD18, and comprises a deletion of amino acid at amino acid position 79 of Erns protein, a deletion of amino acid at amino acid position 171 of Erns protein, a substitution of E to R or K at amino acid position 24 of the E2 protein, or a substitution of E to R or K at amino acid position 24 of the E2 protein and a substitution of G to D at amino acid position 25 of the E2 protein, and optionally further comprises a substitution of S to K, Q or R at amino acid position 14 of the E2 protein and/or a substitution of G to A, R, Q, or E, with A and R being preferred, at amino acid position 22 of the E2 protein.

In one aspect, the recombinant CSFV has a full length nucleotide sequence as shown in SEQ ID NO: 6, or comprises or expresses a polyprotein with the amino acid sequence set forth in SEQ ID NO:24.

In one aspect, the present invention also provides a nucleic acid coding for the recombinant CSFV according to the present invention.

The term “nucleic acid” refers to polynucleotides including DNA molecules, RNA molecules, cDNA molecules or derivatives. The term encompasses single as well as double stranded polynucleotides. The nucleic acid of the present invention encompasses isolated polynucleotides (i.e. isolated from its natural context) and genetically modified forms. Moreover, comprised are also chemically modified polynucleotides including naturally occurring modified polynucleotides such as glycosylated or methylated polynucleotides or artificial modified one such as biotinylated polynucleotides. Further, it is to be understood that the CSFV of the present invention may be encoded by a large number of polynucleotides due to the degenerated genetic code.

In one aspect, the present invention also provides a vector comprising the nucleic acid coding for the recombinant CSFV according to the present invention.

The term “vector” encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the nucleic acid of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. More preferably, the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. For example, the techniques are described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

In one aspect, the present invention also provides an immunogenic composition comprising the recombinant CSFV according to the present invention or the nucleic acid coding for the recombinant CSFV according to the present invention or the vector comprising the nucleic acid coding for the recombinant CSFV according to the present invention. In one aspect, the recombinant CSFV that is part of the immunogenic composition according to the invention is attenuated, preferably as described herein.

The term “immunogenic composition” as used herein refers to a composition that comprises at least one antigen, which elicits an immunological response in the host to which the immunogenic composition is administered. Such immunological response may be a cellular and/or antibody-mediated immune response to the immunogenic composition of the invention. The host is also described as “subject”. Preferably, any of the hosts or subjects described or mentioned herein is an animal.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutically response.

A “protective immunological response” will be demonstrated by either a reduction or lack of clinical signs normally displayed by an infected host, a quicker recovery time and/or a lowered duration of infectivity or lowered pathogen titer in the tissues or body fluids or excretions of the infected host.

An “antigen” as used herein refers to, but is not limited to, components which elicit an immunological response in a host to an immunogenic composition or vaccine of interest comprising such antigen or an immunologically active component thereof. The antigen or immunologically active component may be a microorganism that is whole (in inactivated or modified live form), or any fragment or fraction thereof, which, if administered to a host, can elicit an immunological response in the host. The antigen may be or may comprise complete live organisms in either its original form or as attenuated organisms in a so called modified live vaccine (MLV). The antigen may further comprise appropriate elements of said organisms (subunit vaccines) whereby these elements are generated either by destroying the whole organism or the growth cultures of such organisms and subsequent purification steps yielding in the desired structure(s), or by synthetic processes induced by an appropriate manipulation of a suitable system like, but not restricted to bacteria, insects, mammalian or other species, and optionally by subsequent isolation and purification procedures, or by induction of said synthetic processes in the animal needing a vaccine by direct incorporation of genetic material using suitable pharmaceutical compositions (polynucleotide vaccination). The antigen may comprise whole organisms inactivated by appropriate methods in a so called killed vaccine (KV).

In case where the host displays a protective immunological response such that resistance to new infection will be enhanced and/or the clinical severity of the disease reduced, the immunogenic composition is described as a “vaccine”.

In one aspect, the immunogenic composition of the present invention is a vaccine.

The term “vaccine” as understood herein is a vaccine for veterinary use comprising antigenic substances and is administered for the purpose of inducing a specific and active immunity against a disease provoked by a CSFV infection.

Preferably, the vaccine according to the invention is an attenuated live CSFV vaccine, comprising a live attenuated CSFV, preferably as described herein, eliciting a protective immune response in the host animal, but does not invoke the viral disease due to a mutation in its genome. Live attenuated vaccines have the advantage over inactivated vaccines that they mimic the natural infection more closely. As a consequence they provide in general a higher level of protection than their inactivated counterparts. The attenuated CSFV as described herein, confer active immunity that may be transferred passively via maternal antibodies against the immunogens it contains and sometimes also against antigenically related organisms. A vaccine of the invention refers to a vaccine as defined above, wherein one immunologically active component is a CSFV or of pestiviral origin or derived from a nucleotide sequence that is more than 70% homologous to any known pestivirus sequence (sense or antisense). However, the present invention also relates to vaccines comprising inactivated CSFV according to the present invention.

A vaccine may additionally comprise further components typical to pharmaceutical compositions.

Additional components to enhance the immune response are constituents commonly referred to as “adjuvants”, like e.g. aluminiumhydroxide, mineral or other oils or ancillary molecules added to the vaccine or generated by the body after the respective induction by such additional components, like but not restricted to interferons, interleukins or growth factors.

In one aspect of the present invention, the at least one mutation within the 6B8 epitope of the E2 protein as defined herein, such as for example a substitution at amino acid position 24 of the E2 protein, a substitution at amino acid positions 24/25 of the E2 protein, a substitution at amino acid position 14 of the E2 protein, and/or a substitution at amino acid position 22 of the E2 protein, is used as a marker.

The term “marker” as used herein refers to the mutant 6B8 epitope according to the present invention. The mutant 6B8 epitope according to the present invention is different from the 6B8 epitope sequence of a wildtype CSFV (6B8 epitope that has not been genetically modified). Thus, the mutant 6B8 epitope according to the present invention allows the differentiation of naturally infected animals having a non-mutated 6B8 epitope from vaccinated animals having a mutant 6B8 epitope according to the present invention by exemplary immuno tests and/or genomic analytical tests.

In one aspect of the invention, the immunogenic composition of the present invention is a marker vaccine or a DIVA (differentiation between infected and vaccinated animals) vaccine.

The term “marker vaccine” or “DIVA (differentiation between infected and vaccinated animals)” refers to a vaccine having a marker as set forth above. Thus, a marker vaccine can be used for differentiating a vaccinated animal from a naturally infected animal. The immunogenic composition of the present invention acts as a marker vaccine because, in contrast to infection with wildtype CSFV, in animals vaccinated with the CSFV of the present invention the substituted 6B8 epitope according to the present invention can be specifically detected. By exemplary immuno tests and/or genomic analytical tests the substituted 6B8 epitope according to the present invention can be differentiated from the 6B8 epitope sequence of a wildtype CSFV (a 6B8 epitope that has not been genetically modified). Finally, the marker epitope should be specific for the pathogen in order to avoid false-positive serological results which are induced by other organisms that may appear in livestock.

However, as shown in the Examples, the 6B8 epitope is evolutionarily conserved (sequence alignment) and specific for CSFV (6B8 mAb does not bind to BVDV). Thus, the substituted 6B8 epitope according to the present invention is highly suitable to be used in a marker vaccine.

Preferably, the marker vaccine according to the invention is an attenuated live vaccine, comprising a live attenuated CSFV eliciting a protective immune response in the host animal, but does not invoke the viral disease due to a mutation in its genome. Live attenuated vaccines have the advantage over inactivated vaccines that they mimic the natural infection more closely. As a consequence they provide in general a higher level of protection than their inactivated counterparts. Suitable live attenuated CSFV marker vaccines according to the invention comprise the mutation within the Erns and/or Npro protein, and a mutant 6B8 epitope, each as disclosed herein.

However this does not necessarily mean that the vaccine must replicate in the target animal in order to act as a vaccine. A recombinant CSFV according to the present invention inherently carries its marker-characteristics (e.g. the mutant 6B8 epitope according to the present invention). Therefore, the virus functions as a marker vaccine in the target animal regardless if it replicates in the target animal or not. Thus, the present invention also relates to marker vaccines comprising inactivated CSFV according to the present invention.

(Non-marker-) live attenuated viruses of CSFV have been described in the art and are even commercially available. And thus, such viruses constitute a very suitable starting material for the construction of viruses according to the invention, i.e. replication-competent CSFV having the mutation in the 6B8 epitope according to the present invention. Such viruses do inherently behave attenuated compared to their wild-type counterparts, and they can thus be used as a basis for marker viruses in a marker vaccine.

A major advantage of an efficacious marker vaccine is that it allows the detection of pigs acutely infected or infected some time (for example at least ca. 3 weeks) before taking samples in a vaccinated pig population, and thus offers the possibility to monitor the spread or re-introduction of CSFV in a pig population. Thus, it makes it possible to declare, with a certain level of confidence, that a vaccinated pig population is free of CSFV on the basis of laboratory test results.

The marker vaccine of the present invention is ideally suited for an emergency vaccination in the case of swine fever detection or outbreak. The marker vaccine facilitates fast and effective administration and allows discrimination between animals infected with the field virus (disease-associated) and vaccinated animals.

In one aspect of the present invention, the animals treated with the immunogenic composition of the present invention can be differentiated from animals infected with naturally occurring swine fever virus via analysis of samples obtained from said animals using immuno tests and/or genomic analytical tests.

The term “sample” refers to a sample of a body fluid, to a sample of separated cells or to a sample from a tissue or an organ. Samples of body fluids can be obtained by well-known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the bodyfluids or the tissues or organs by separating techniques such as centrifugation or cell sorting.

The term “obtained” may comprise an isolation and/or purification step known to the person skilled in the art, preferably using precipitation, columns etc.

The term “immuno tests” and “genomic analytical tests” are specified below. However, the analysis of said “immuno tests” and “genomic analytical tests”, respectively, is the basis for differentiating animals vaccinated with the immunogenic composition according to the present invention and animals infected with the naturally occurring (disease-associated) swine fever virus.

In one aspect of the present invention said immunogenic composition is formulated for a single-dose administration.

Advantageously, the experimental data provided by the present invention disclose that a single dose administration of the immunogenic composition of the present invention reliably and effectively stimulated a protective immune response. Thus, in one aspect of the invention said immunogenic composition is formulated for and effective by a single-dose administration.

Also, the invention provides the use of the immunogenic composition of the present invention for use as a medicament.

In one aspect, the invention provides a method of preventing and/or treating diseases associated with CSFV in an animal, the method comprising the step of administering the immunogenic composition according to the invention to an animal in need thereof. In one aspect, the disease associated with CSFV is CSF.

The present invention also relates to a method for immunizing an animal, comprising administering to such animal any of the immunogenic compositions according to the present invention.

The present invention also relates to a method for immunizing an animal, comprising a single administering to such animal any of the immunogenic compositions according to the present invention.

Preferably, the method for immunizing an animal is effective by the single administration of the immunogenic compositions according to the present invention to such animal

The term “immunizing” relates to an active immunization by the administration of an immunogenic composition to an animal to be immunized, thereby causing an immunological response against the antigen included in such immunogenic composition.

The immunization results in lessening of the incidence of the particular CSFV infection in a herd or in the reduction in the severity of clinical signs caused by or associated with the particular CSFV infection. Preferably, the immunization results in lessening of the incidence of the particular CSFV infection in a herd or in the reduction in the severity of clinical signs caused by or associated with the particular CSFV infection by a single administration of the immunogenic composition according to the present invention.

According to one aspect of the invention, the immunization of an animal in need with the immunogenic compositions as provided herewith, results in preventing infection of a subject by CSFV infection, preferably by a single administration of the immunogenic composition according to the present invention. Even more preferably, immunization results in an effective, long-lasting, immunological-response against CSFV infection. It will be understood that the said period of time will last more than 2 months, preferably more than 3 months, more preferably more than 4 months, more preferably more than 5 months, more preferably more than 6 months. It is to be understood that immunization may not be effective in all animals immunized. However, the term requires that a significant portion of animals of a herd are effectively immunized.

Preferably, a herd of animals is envisaged in this context which normally, i.e. without immunization, would develop clinical signs normally caused by or associated with a CSFV infection. Whether the animals of a herd are effectively immunized can be determined without further ado by the person skilled in the art. Preferably, the immunization shall be effective if clinical signs in at least 33%, at least 50%, at least 60%, at least 70%, at least 80%, or at least 90% of the animals of a given herd are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to animals that are either not immunized or immunized with an immunogenic composition that was available prior to the present invention but subsequently infected by CSFV.

In one aspect of the present invention, the animal is swine. In one aspect the animal is a piglet. Piglets are normally younger than 3 to 4 weeks of age. In one aspect the piglets are vaccinated between 1 to 4 weeks of age. In one aspect the animal is a sow. In one aspect the animal is a pregnant sow.

In one aspect of the present invention, the immunogenic composition is administered intradermal, intratracheal, intravaginal, intramuscular, intranasal, intravenous, intraarterial, intraperitoneal, oral, intrathecal, subcutaneous, intracutaneous, intracardial, intralobal, intramedullar, intrapulmonary, and combinations thereof. However, depending on the nature and mode of action of a compound, the immunogenic composition may be administered by other routes as well.

The present invention also provides a method of reducing the incidence of or severity in an animal of one or more clinical signs associated with CSF, the method comprising the step of administering the immunogenic composition according to the present invention to an animal in need thereof, wherein the reduction of the incidence of or the severity of the one or more clinical signs is relative to an animal not receiving the immunogenic composition. Preferably, the method comprises the administration of a single dose of the immunogenic composition and is effective in reduction of the incidence of or the severity of the one or more clinical signs by such single administration of the immunogenic composition.

The term “clinical signs” as used herein refers to signs of infection of an animal from CSFV. The clinical signs are defined further below. However, the clinical signs also include but are not limited to clinical signs that are directly observable from a live animal. Examples for clinical signs that are directly observable from a live animal include nasal and ocular discharge, lethargy, coughing, wheezing, thumping, elevated fever, weight gain or loss, dehydration, diarrhea, joint swelling, lameness, wasting, paleness of the skin, unthriftiness, and the like. Mittelholzer et al. (Vet. Microbiol., 2000. 74(4): p. 293-308) developed a checklist for the determination of the clinical scores in CSF animal experiments. This checklist contains the parameters liveliness, body tension, body shape, breathing, walking, skin, eyes/conjunctiva, appetite, defecation and leftovers in feeding through.

Preferably, clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to subjects that are either not treated or treated with an immunogenic composition that was available prior to the present invention but subsequently infected by CSFV.

In one aspect of the invention the immunogenic composition is administered once only and is efficacious by such single-dose administration.

As shown in the Examples the immunogenic composition as provided herein has been proven to be efficacious after the administration of a single dose of said immunogenic composition to an animal of need.

However, while the single dose administration is preferred, the immunogenic composition can also be administered twice or several times, with a first dose being administered prior to the administration of a second (booster) dose. Preferably, the second dose is administered at least 15 days after the first dose. More preferably, the second dose is administered between 15 and 40 days after the first dose. Even more preferably, the second dose is administered at least 17 days after the first dose. Still more preferably, the second dose is administered between 17 and 30 days after the first dose. Even more preferably, the second dose is administered at least 19 days after the first dose. Still more preferably, the second dose is administered between 19 and 25 days after the first dose. Most preferably the second dose is administered at least 21 days after the first dose. In a preferred aspect of the two-time administration regimen, both the first and second doses of the immunogenic composition are administered in the same amount. In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned above.

The amount of the CSFV to be administered may be an amount of the virus that elicits or is able to elicit an immune response in an animal, to which the dose of the virus is administered. If an inactivated virus or a modified live virus preparation is used, an amount of the vaccine containing about 10² to about 10⁹ TCID₅₀ (tissue culture infective dose 50% end point), more preferably 10⁴ to about 10⁸ TCID₅₀, and still more preferably from about 10⁴ to about 10⁶ TCID₅₀ per dose may be recommended.

Preferably, the single-dose has a total volume between about 0.5 ml and 2.5 ml, more preferably between about 0.6 ml and 2.0 ml, even more preferably between about 0.7 ml and 1.75 ml, still more preferably between about 0.8 ml and 1.5 ml, even more preferably between about 0.9 ml and 1.25 ml, with a single 1.0 ml dose being the most preferred.

In one aspect of the invention the one or more clinical signs are selected from the group consisting of: respiratory distress, labored breathing, coughing, sneezing, rhinitis, tachypnea, dyspnea, pneumonia, red/blue discolouration of the ears and vulva, jaundice, lymphocytic infiltrates, lymphadenopathy, hepatitis, nephritis, anorexia, fever, lethargy, agalatia, diarrhea, nasal extrudate, conjunctivitis, progressive weight loss, reduced weight gain, paleness of the skin, gastric ulcers, macroscopic and microscopic lesions on organs and tissues, lymphoid lesions, mortality, virus induced abortion, stillbirth, malformation of piglets, mummification and combinations thereof.

In one aspect, the present invention also provides a method of marking a CSFV vaccine comprising introducing into a CSFV at least one mutation within the 6B8 epitope of the E2 protein, wherein the (unmodified) 6B8 epitope is specifically recognized by the 6B8 monoclonal antibody. In one aspect the 6B8 monoclonal antibody is produced by a hybridoma deposited at CCTCC under the accession number CCTCC C2018120, or the 6B8 monoclonal antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or the 6B8 monoclonal antibody comprises the CDRs of the monoclonal antibody produced by a hybridoma deposited at CCTCC under the accession number CCTCC C2018120, or the 6B8 monoclonal antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30. In one aspect, the mutated 6B8 epitope is one of the modified 6B8 epitopes as disclosed herein.

In one aspect, said mutation is a substitution at amino acid position 24 of the E2 protein, a substitution at amino acid positions 24/25 of the E2 protein, a substitution at amino acid position 14 of the E2 protein and/or a substitution at amino acid position 22 of the E2 protein. In one aspect, said mutation is a substitution at amino acid position 24 of the E2 protein and a substitution at amino acid position 25 of the E2 protein. In one aspect, said mutation is a substitution at amino acid position 24 of the E2 protein, and a substitution at amino acid position 14 of the E2 protein. In one aspect, said mutation is a substitution at amino acid position 24 of the E2 protein, a substitution at amino acid position 25 of the E2 protein, and a substitution at amino acid position 14 of the E2 protein. In one aspect, said mutation is a substitution at amino acid position 24 of the E2 protein, and a substitution at amino acid position 22 of the E2 protein. In one aspect, said mutation is a substitution at amino acid position 24 of the E2 protein, a substitution at amino acid position 25 of the E2 protein, and a substitution at amino acid position 22 of the E2 protein. In one aspect, said mutation is a substitution at amino acid position 24 of the E2 protein, a substitution at amino acid position 14 of the E2 protein and a substitution at amino acid position 22 of the E2 protein. In one aspect, said mutation is a substitution at amino acid position 24 of the E2 protein, a substitution at amino acid position 25 of the E2 protein, a substitution at amino acid position 14 of the E2 protein and a substitution at amino acid position 22 of the E2 protein.

In one aspect, the amino acid at position 24 of the E2 protein is substituted to R or K, the amino acid at positions 24/25 of the E2 protein is substituted to R/D or K/D, the amino acid at position 14 of the E2 protein is substituted to K, Q or R, and/or the amino acid at position 22 of the E2 protein is substituted to A, R, Q or E, with A and R being preferred. In one aspect, the amino acid at position 24 of the E2 protein is substituted to R or K, and the amino acid at position 25 of the E2 protein is substituted to D. In one aspect, the amino acid at position 24 of the E2 protein is substituted to R or K, and the amino acid at position 14 of the E2 protein is substituted to K, Q or R. In one aspect, the amino acid at position 24 of the E2 protein is substituted to R or K, the amino acid at position 25 of the E2 protein is substituted to D, and the amino acid at position 14 of the E2 protein is substituted to K, Q or R.

In one aspect, the amino acid at position 24 of the E2 protein is substituted to R or K, and the amino acid at position 22 of the E2 protein is substituted to A, R, Q or E, with A and R being preferred. In one aspect, the amino acid at position 24 of the E2 protein is substituted to R or K, the amino acid at position 25 of the E2 protein is substituted to D, and the amino acid at position 22 of the E2 protein is substituted to A, R, Q or E, with A and R being preferred.

In one aspect, the amino acid at position 24 of the E2 protein is substituted to R or K, the amino acid at position 14 of the E2 protein is substituted to K, Q or R, and the amino acid at position 22 of the E2 protein is substituted to A, R, Q or E, with A and R being preferred. In one aspect, the amino acid at position 24 of the E2 protein is substituted to R or K, the amino acid at position 25 of the E2 protein is substituted to D, the amino acid at position 14 of the E2 protein is substituted to K, Q or R, and the amino acid at position 22 of the E2 protein is substituted to A, R, Q or E, with A and R being preferred.

In one aspect, said mutation is a substitution of E or G to R or K at amino acid position 24 of the E2 protein, a substitution of E or G to R or K at amino acid position 24 of the E2 protein and a substitution of G to D at amino acid position 25 of the E2 protein, a substitution of S to K, Q or R at amino acid position 14 of the E2 protein and/or a substitution of G to A, R, Q or E, with A and R being preferred at amino acid position 22 of the E2 protein. In one aspect, said mutation is a substitution of E or G to R or K at amino acid position 24 of the E2 protein, and a substitution of G to D at amino acid position 25 of the E2 protein. In one aspect, said mutation is a substitution of E or G to R or K at amino acid position 24 of the E2 protein, and a substitution of S to K, Q or R at amino acid position 14 of the E2 protein. In one aspect, said mutation is a substitution of E or G to R or K at amino acid position 24 of the E2 protein, a substitution of G to D at amino acid position 25 of the E2 protein, and a substitution of S to K, Q or R at amino acid position 14 of the E2 protein. In one aspect, said mutation is a substitution of E or G to R or K at amino acid position 24 of the E2 protein, and a substitution of G to A, R, Q or E, with A and R being preferred at amino acid position 22 of the E2 protein. In one aspect, said mutation is a substitution of E or G to R or K at amino acid position 24 of the E2 protein, a substitution of G to D at amino acid position 25 of the E2 protein, and a substitution of G to A, R, Q or E, with A and R being preferred at amino acid position 22 of the E2 protein.

In one aspect, said mutation is a substitution of E or G to R or K at amino acid position 24 of the E2 protein, a substitution of S to K, Q or R at amino acid position 14 of the E2 protein and a substitution of G to A, R, Q or E, with A and R being preferred at amino acid position 22 of the E2 protein. In one aspect, said mutation is a substitution of E or G to R or K at amino acid position 24 of the E2 protein, a substitution of G to D at amino acid position 25 of the E2 protein, a substitution of S to K, Q or R at amino acid position 14 of the E2 protein and a substitution of G to A, R, Q or E, with A and R being preferred at amino acid position 22 of the E2 protein.

The term “marking” as used herein refers to the introduction of a “marker” as defined above into a CSFV or CSFV vaccine. Thus, it has to be understood that the method of the present invention also refers to the marking of a CSFV and is not restricted to a method of making a CSFV vaccine.

Thus, a “marker vaccine” or a “DIVA vaccine” as defined above may be produced by marking a CSFV vaccine according to the method of the present invention. In one aspect of the present invention, the CSFV vaccine is an attenuated vaccine.

Attenuated CSFV vaccines already have been defined above. Further, it has to be understood that the method according to the present invention is not restricted to the production of attenuated CSFV vaccines. In contrast, as set forth above, a virus functions as a marker vaccine in the target animal regardless if it replicates in the target animal or not. Thus, the present invention also relates to marker vaccines comprising inactivated CSFV according to the present invention.

In a further aspect, the present invention also provides a method of differentiating animals infected with CSFV from animals vaccinated with the immunogenic composition according to the present invention, comprising

a) obtaining a sample, and

b) testing said sample in an immuno test and/or genomic analytical test.

The term “immuno test” refers to a test comprising an antibody specific for the 6B8 epitope of the E2 protein of the CSFV. The antibody may be specific for the mutant 6B8 epitope according to the present invention or for the 6B8 epitope of a wildtype CSFV (6B8 epitope that has not been genetically modified). However, the term “immuno test” does also refer to a test comprising mutant 6B8 epitope peptides according to the present invention or 6B8 epitope peptides of a wildtype CSFV (6B8 epitope that has not been genetically modified). Examples of immuno tests include any enzyme-immunological or immunochemical detection method such as ELISA (enzyme linked immunosorbent assay), EIA (enzyme immunoassay), RIA (radioimmunoassay), sandwich enzyme immune tests, fluorescent antibody test (FAT), electrochemiluminescence sandwich immunoassays (ECLIA), dissociation-enhanced lanthanide fluoro immuno assay (DELFIA) or solid phase immune tests, immunofluorescent test (IFT), immunohistological staining, Western blot analysis or any other suitable method available to technicians skilled in the art. Depending upon the assay used, the antigens or the antibodies can be labeled by an enzyme, a fluorophore or a radioisotope. See, e.g., Coligan et al. Current Protocols in Immunology, John Wiley & Sons Inc., New York, N.Y. (1994); and Frye et al., Oncogen 4: 1153-1157, 1987.

Preferably, an antibody specific for the 6B8 epitope of a wildtype CSFV is used to detect CSFV antigen in serum cells (such as leucocytes) or cryostat sections of isolated organs (such as tonsils, spleen, kidney, lymph nodes, distal portions of the ileum) from an animal (such as a pig) that is suspected to be infected with wildtype CSFV or that is vaccinated with a vaccine comprising a recombinant CSFV according to the invention. In such a case, only the sample of an animal infected with wildtype CSFV will show positive results by said 6B8 epitope specific antibody. In contrast, the sample of an animal vaccinated with the vaccine comprising a recombinant CSFV of the present invention will show no results by said 6B8 epitope specific antibody due to the mutation within the 6B8 epitope according to the present invention. In an alternative test, CSFV is isolated from, for example, organs (such as the tonsils of an animal) or serum cells (such as leukoyctes) infected, suspected to be infected with wildtype CSFV or vaccinated animals and incubated with a suitable cell line (such as SK-6 cells or PK-15 cells) for infection of the cells with the virus. The replicated virus is subsequently detected in the cells using 6B8 epitope specific antibodies that differentiate between the field (wildtype, disease associated) CSFV and the recombinant CSFV according to the invention. Further, peptides could be used to block unspecific cross-reactivity. Moreover, antibodies specific for other epitopes of the wildtype CSFV could be used as a positive control.

More preferably, an ELISA is used, wherein the antibody specific for the 6B8 epitope of a wildtype CSFV (6B8 epitope that has not been genetically modified) is cross-linked to micro-well assay plates for differentiating between infected pigs from pigs vaccinated with the vaccine according to the present invention. Said cross-linking preferably is performed through an anchor protein such as, for example, poly-L-lysine. ELISAs employing such cross-linking are in general more sensitive when compared to ELISAs employing a passively coated technique. The wildtype (disease associated) CSFV binds to the antibody specific for the 6B8 epitope of a wildtype CSFV (6B8 epitope that has not been genetically modified). The detection of the binding of the wildtype CSFV to the antibody specific for the 6B8 epitope of a wildtype CSFV can be performed by a further antibody specific for CSFV. In such a case, only the sample of the infected pig will show positive results by the 6B8 epitope specific antibody. In contrast, the recombinant CSFV of a pig vaccinated with the vaccine according to the present invention will express only the mutant 6B8 epitope, and, thus, will not bind to the antibody specific for the 6B8 epitope of a wildtype CSFV (6B8 epitope that has not been genetically modified) that has been cross-linked to the micro-well assay plates. Further, peptides could be used to block unspecific cross-reactivity. Moreover, antibodies specific for other epitopes of the wildtype CSFV could be used as a positive control.

Alternatively, the micro-well assay plates may be cross-linked with an antibody specific for CSFV other than the antibody specific for the 6B8 epitope of a wildtype CSFV (6B8 epitope that has not been genetically modified). The wildtype (disease associated) CSFV binds to the cross linked antibody. The detection of the binding of the wildtype CSFV to the cross linked antibody can be performed by the antibody specific for the 6B8 epitope of a wildtype CSFV (6B8 epitope that has not been genetically modified).

As already set forth above the 6B8 epitope is evolutionarily conserved and specific for wildtype CSFV.

Therefore, more preferably, an ELISA is used for detecting in the sample antibodies that are directed against the mutant 6B8 epitope according to the present invention or the 6B8 epitope of a wildtype CSFV (6B8 epitope that has not been genetically modified). Such a test comprises mutant 6B8 epitope peptides according to the present invention or the 6B8 epitope peptides of a wildtype CSFV (6B8 epitope that has not been genetically modified).

Such a test could e.g. comprise wells with a substituted 6B8 epitope according to the present invention or the 6B8 epitope of a wildtype CSFV (6B8 epitope that has not been genetically modified) cross-linked to micro-well assay plates. Said cross-linking preferably is performed through an anchor protein such as, for example, poly-L-lysine. Expression systems for obtaining a mutant or wildtype 6B8 epitope are well known to the person skilled in the art. Alternatively, said 6B8 epitopes could be chemically synthesized. It has to be understood that although the mutant or wildtype 6B8 epitope as such can be used in a test according to the invention, it can be convenient to use a protein comprising the complete E2 protein or a fragment of the E2 protein comprising the said 6B8 epitope, instead of the relatively short epitope as such. Especially when the epitope is for example used for the coating of a well in a standard ELISA test, it may be more efficient to use a larger protein comprising the epitope, for the coating step.

Animals vaccinated with the vaccine comprising a recombinant CSFV according to the present invention have not raised antibodies against the wild-type 6B8 epitope. However, such animals have raised antibodies against the substituted 6B8 epitope according to the present invention. As a consequence, no antibodies bind to a well coated with the wildtype 6B8 epitope. In contrast, if a well has been coated with the mutant 6B8 epitope according to the present invention, antibodies bind to said mutant 6B8 epitope.

Animals infected with the wildtype CSFV will however have raised antibodies against the wildtype epitope of CSFV. However, such animals have not raised antibodies against the mutant 6B8 epitope according to the present invention. As a consequence, no antibodies bind to a well coated with the mutant 6B8 epitope according to the present invention. In contrast, if a well has been coated with the wildtype 6B8 epitope, antibodies bind to the wildtype 6B8 epitope.

The binding of the antibodies to the mutant 6B8 epitope according to the present invention or the 6B8 epitope of a wildtype CSFV (6B8 epitope that has not been genetically modified) can be done by methods well known to the person skilled in the art.

Preferably, the ELISA is a sandwich type ELISA. More preferably, the ELISA is a competitive ELISA. Most preferably, the ELISA is a double competitive ELISA. However, the different ELISA techniques are well known to the person skilled in the art. ELISA have been described exemplary by Wensvoort G. et al., 1988 (Vet. Microbiol. 17(2): 129-140), by Robiolo B. et al., 2010 (J. Virol. Methods. 166(1-2): 21-27) and by Colijn, E. O. et al., 1997 (Vet. Microbiology 59: 15-25).

The term “genomic analytical test” refers to a genomic analytical method based upon the polymerase chain reaction (PCR), reverse transcription polymerase chain reaction (RT-PCR), real-time PCR (r-PCR) or real time reverse transcription PCR (rRT-PCR), Templex-PCR, nucleic-acid sequence based amplification (NASBA), and isothermal amplification methods using polymerases and specific oligonucleotides as primers. The aforementioned amplification methods are well known in the art.

Preferably, the test for differentiating an animal that is infected with wildtype CSFV or vaccinated with a recombinant CSFV of the invention is provided by RNA isolation of the CSFV and reverse transcription followed by amplification of the cDNA. The cDNA is then sequenced for detecting whether the 6B8 epitope is intact and refers to a wildtype CSFV. In such a case the pig is infected with the wildtype CSFV. However, if the sequence of the 6B8 epitope is substituted according to the present invention, the animal has been vaccinated with the vaccine of the present invention.

Further, when using any real time based technique primers and/or probes may be used recognizing either the modified (mutants according to the present invention) and/or disease-associated (wildtype) viral nucleotide sequence of the 6B8 epitope. However, such methods are well known in the art.

In one aspect of the present invention the immuno test comprises testing whether antibodies specifically recognizing the intact 6B8 epitope of the CSFV E2 protein are binding to the CSFV E2 protein in the sample. In one aspect of the present invention the immuno test comprises testing whether an antibody specifically recognizing a 6B8 epitope of the CSFV E2 protein is present in the sample, and/or testing whether an antibody specifically recognizing a mutated 6B8 epitope of the CSFV E2 protein is present in the sample. Such a mutated 6B8 epitope comprises mutation(s) in the 6B8 epitope as disclosed herein.

In one aspect of the present invention the immuno test is an EIA (enzyme immunoassay) or ELISA (enzyme linked immunosorbent assay). In one aspect of the present invention the ELISA is an indirect ELISA, Sandwich ELISA, a competitive ELISA or double competitive ELISA, preferably a double competitive ELISA. In one aspect of the present invention the genomic analytical test is a PCR (polymerase chain reaction), RT-PCR (reverse transcriptase polymerase chain reaction) or real time PCR (polymerase chain reaction). In one aspect of the present invention the sample is a serum sample. In one aspect of the present invention the animal is swine.

In one aspect, the invention relates to an antibody or an antigen binding fragment thereof, wherein the antibody specifically recognizes the 6B8 epitope as defined herein above. In one aspect, the invention relates to an antibody or an antigen binding fragment thereof, wherein the antibody is produced by a hybridoma deposited at CCTCC under the accession number CCTCC C2018120. In one aspect, the invention relates to an antibody or an antigen binding fragment thereof, wherein the antibody comprises a heavy chain variable region (V_{H}) having an amino acid sequence as set forth in SEQ ID NO: 9 and a light chain variable region (V_{L}) having an amino acid sequence as set forth in SEQ ID NO: 10. In one aspect, the invention relates to an antibody or an antigen binding fragment thereof, wherein the antibody comprises the CDRs of the monoclonal antibody produced by a hybridoma deposited at CCTCC under the accession number CCTCC C2018120. In one aspect, the invention relates to an antibody or an antigen binding fragment thereof, wherein the antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

Said antibody may be also designated “6B8” or “6B8 monoclonal antibody” and is capable of specifically binding to E2 protein of classical swine fever virus (CSFV), and thus can be used for developing a marker or DIVA vaccine against CSFV.

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

The term “variable region” as used herein means an immunoglobulin domain essentially consisting of four “framework regions” which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1” or “CDR1”; as “complementarity determining region 2” or “CDR2”; and as “complementarity determining region 3” or “CDR3”, respectively. Thus, the general structure or sequence of an immunoglobulin variable region can be indicated as follows: FR1-CDR1-FR2-CDR2-FR3-CDR3-FR4. VH or V_{H} refers to a heavy chain variable region, and VL or V_{L} refers to a light chain variable region. Similarly, VH CDR1, VH CDR2 and VH CDR3 refer to CDR1, CDR2 and CDR3 of a heavy chain variable region, respectively. VL CDR1, VL CDR2 and VL CDR3 refer to CDR1, CDR2 and CDR3 of a light chain variable region, respectively.

As used herein, an “antibody fragment” or “antigen-binding fragment” of an antibody refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically, e.g. recombinantly produced derivatives. An antibody fragment is included among antibodies. Examples of antibody fragments include, but are not limited to, Fab, Fab′, F(ab′)2, single-chain Fv (scFv), Fv, dsFv, diabody, Fd and Fd′ fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antigen-binding fragment includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen.

In one aspect, the present invention also provides a mutant E2 protein of CSFV for use in the DIVA method of the invention, which comprises at least one mutation within the 6B8 epitope. The 6B8 epitope is already defined above.

In one aspect, the mutant E2 protein for use in the DIVA method of the invention comprises a substitution at amino acid position 24 of the E2 protein, a substitution at amino acid positions 24/25 of the E2 protein, a substitution at amino acid position 14 of the E2 protein, and/or a substitution at amino acid position 22 of the E2 protein.

In one aspect, the mutant E2 protein for use in the DIVA method of the invention comprises a substitution at amino acid position 24 of the E2 protein and a substitution at amino acid position 25 of the E2 protein.

In one aspect, the mutant E2 protein for use in the DIVA method of the invention comprises a substitution at amino acid position 24 of the E2 protein and a substitution at amino acid position 14 of the E2 protein. In one aspect, the mutant E2 protein for use in the DIVA method of the invention comprises a substitution at amino acid position 24 of the E2 protein, a substitution at amino acid position 25 of the E2 protein, and a substitution at amino acid position 14 of the E2 protein.

In one aspect, the mutant E2 protein for use in the DIVA method of the invention comprises a substitution at amino acid position 24 of the E2 protein, and a substitution at amino acid position 22 of the E2 protein. In one aspect, the mutant E2 protein for use in the DIVA method of the invention comprises a substitution at amino acid position 24 of the E2 protein, a substitution at amino acid position 25 of the E2 protein, and a substitution at amino acid position 22 of the E2 protein.

In one aspect, the mutant E2 protein for use in the DIVA method of the invention comprises a substitution at amino acid position 14 of the E2 protein, and a substitution at amino acid position 22 of the E2 protein.

In one aspect, the mutant E2 protein for use in the DIVA method of the invention comprises a substitution at amino acid position 24 of the E2 protein, a substitution at amino acid position 14 of the E2 protein and a substitution at amino acid position 22 of the E2 protein. In one aspect, the mutant E2 protein for use in the DIVA method of the invention comprises a substitution at amino acid position 24 of the E2 protein, a substitution at amino acid position 25 of the E2 protein, a substitution at amino acid position 14 of the E2 protein and a substitution at amino acid position 22 of the E2 protein.

In one aspect, the amino acid at position 24 of the E2 protein is substituted to R or K, the amino acid at position 24/25 of the E2 protein is substituted to R/D or K/D, the amino acid at position 14 of the E2 protein is substituted to K, Q or R, and/or the amino acid at position 22 of the E2 protein is substituted to A, R, Q or E, with A and R being preferred.

In one aspect, the amino acid at position 24 of the E2 protein is substituted to R or K, and the amino acid at position 25 of the E2 protein is substituted to D.

In one aspect, the amino acid at position 24 of the E2 protein is substituted to R or K, and the amino acid at position 14 of the E2 protein is substituted to K, Q or R. In one aspect, the amino acid at position 24 of the E2 protein is substituted to R or K, the amino acid at position 25 of the E2 protein is substituted to D, and the amino acid at position 14 of the E2 protein is substituted to K, Q or R.

In one aspect, the amino acid at position 24 of the E2 protein is substituted to R or K, and the amino acid at position 22 of the E2 protein is substituted to A, R, Q or E, with A and R being preferred.

In one aspect, the amino acid at position 24 of the E2 protein is substituted to R or K, the amino acid at position 25 of the E2 protein is substituted to D, and the amino acid at position 22 of the E2 protein is substituted to A, R, Q or E, with A and R being preferred.

In one aspect, the amino acid at position 14 of the E2 protein is substituted to K, Q or R, and the amino acid at position 22 of the E2 protein is substituted to A, R, Q or E, with A and R being preferred.

In one aspect, the amino acid at position 24 of the E2 protein is substituted to R or K, the amino acid at position 14 of the E2 protein is substituted to K, Q or R, and the amino acid at position 22 of the E2 protein is substituted to A, R, Q or E, with A and R being preferred. In one aspect, the amino acid at position 24 of the E2 protein is substituted to R or K, the amino acid at position 25 of the E2 protein is substituted to D, the amino acid at position 14 of the E2 protein is substituted to K, Q or R, and the amino acid at position 22 of the E2 protein is substituted to A, R, Q or E, with A and R being preferred.

In one aspect, the mutant E2 protein comprises a substitution of E or G to R or K at amino acid position 24 of the E2 protein, a substitution of E or G to R or K at amino acid position 24 of the E2 protein and a substitution of G to D at amino acid position 25 of the E2 protein, a substitution of S to K, Q or R at amino acid position 14 of the E2 protein, and/or a substitution of G to A, R, Q or E, with A and R being preferred, at amino acid position 22 of the E2 protein.

In one aspect, said mutation is a substitution of E or G to R or K at amino acid position 24 of the E2 protein, and a substitution of G to D at amino acid position 25 of the E2 protein.

In one aspect, said mutation is a substitution of E or G to R or K at amino acid position 24 of the E2 protein, and a substitution of S to K, Q or R at amino acid position 14 of the E2 protein. In one aspect, said mutation is a substitution of E or G to R or K at amino acid position 24 of the E2 protein, a substitution of G to D at amino acid position 25 of the E2 protein, and a substitution of S to K, Q or R at amino acid position 14 of the E2 protein.

In one aspect, said mutation is a substitution of E or G to R or K at amino acid position 24 of the E2 protein, and a substitution of G to A, R, Q or E, with A and R being preferred, at amino acid position 22 of the E2 protein. In one aspect, said mutation is a substitution of E or G to R or K at amino acid position 24 of the E2 protein, a substitution of G to D at amino acid position 25 of the E2 protein, and a substitution of G to A, R, Q or E, with A and R being preferred, at amino acid position 22 of the E2 protein.

In one aspect, said mutation is a substitution of E or G to R at amino acid position 24 of the E2 protein, a substitution of S to K, Q or R at amino acid position 14 of the E2 protein and a substitution of G to A, R, Q or E, with A and R being preferred, at amino acid position 22 of the E2 protein. In one aspect, said mutation is a substitution of E or G to R at amino acid position 24 of the E2 protein, a substitution of G to D at amino acid position 25 of the E2 protein, a substitution of S to K, Q or R at amino acid position 14 of the E2 protein and a substitution of G to A, R, Q or E, with A and R being prefer, at amino acid position 22 of the E2 protein.

The mutant E2 protein of the invention is especially suitable for use in the DIVA method of the invention, for example, through a double competition ELISA.

In one aspect, the mutant E2 protein of CSFV for use in the DIVA method of the invention may be a truncation of the full length E2 protein, for example, a trans-membrane region may be deleted.

In one aspect, the present invention provides a kit for differentiating animals infected with CSFV from animals vaccinated with the immunogenic composition of the invention, which comprises the antibody of the invention or an antigen-binding fragment thereof, a mutant E2 protein of the invention, and/or a wildtype E2 polypeptide of CSFV comprising the 6B8 epitope as defined herein. The kit may also contain instructions for use.

In one aspect, the present invention provides an attenuated CSFV, wherein the attenuated CSFV is derived from the field strain QZ07 or the field strain GD18 or C-strain.

In one aspect, the attenuated CSFV contains at least one mutation in the Erns protein. Preferably such attenuated CSFV has one or more mutations within the 6B8 epitope of the E2 protein as disclosed herein.

Such mutation in the Erns protein can be a deletion of amino acid at amino acid position 79 of Erns protein and/or a deletion of amino acid at amino acid position 171 of Erns protein. Such mutation in the Erns protein can be a deletion of amino acid at amino acid position 79 of Erns protein. Such mutation in the Erns protein can be a deletion of amino acid at amino acid position 171 of Erns protein. Such mutation in the Erns protein can be a deletion of amino acid at amino acid position 79 of Erns protein and a deletion of amino acid at amino acid position 171 of Erns protein.

In one aspect, the attenuated CSFV contains at least one mutation in the Npro protein. Preferably such attenuated CSFV has one or more mutations within the 6B8 epitope of the E2 protein as disclosed herein.

Such mutation in Npro protein can be a deletion of the Npro protein except for the first four amino terminal amino acids. Other modifications may also be introduced for attenuation.

In one aspect, the attenuated CSFV contains at least one mutation in the Erns protein and/or at least one mutation in the Npro protein. Preferably such attenuated CSFV has one or more mutations within the 6B8 epitope of the E2 protein as disclosed herein.

In one aspect, the attenuated CSFV is derived from the field strain QZ07 and contains a deletion of amino acid at amino acid position 79 of Erns protein. Preferably such attenuated CSFV has one or more mutations within the 6B8 epitope of the E2 protein as disclosed herein.

In one aspect, the attenuated CSFV is derived from the field strain QZ07 and contains a deletion of amino acid at amino acid position 79 of Erns protein and a deletion of amino acid at amino acid position 171 of Erns protein. Preferably such attenuated CSFV has one or more mutations within the 6B8 epitope of the E2 protein as disclosed herein.

In one aspect, the attenuated CSFV is derived from the field strain QZ07 and contains a deletion of amino acid at amino acid position 79 of Erns protein and a deletion of the Npro protein except for the first four amino terminal amino acids. Preferably such attenuated CSFV has one or more mutations within the 6B8 epitope of the E2 protein as disclosed herein.

In one aspect, the attenuated CSFV is derived from the field strain QZ07 and contains a deletion of amino acid at amino acid position 79 of Erns protein, a deletion of amino acid at amino acid position 171 of Ems protein and a deletion of the Npro protein except for the first four amino terminal amino acids. Preferably such attenuated CSFV has one or more mutations within the 6B8 epitope of the E2 protein as disclosed herein.

In one aspect, the attenuated CSFV is derived from the field strain GD18 and contains a deletion of amino acid at amino acid position 79 of Erns protein and a deletion of the Npro protein except for the first four amino terminal amino acids. Preferably such attenuated CSFV has one or more mutations within the 6B8 epitope of the E2 protein as disclosed herein.

In one aspect, the attenuated CSFV is derived from the field strain GD18 and contains a deletion of amino acid at amino acid position 79 of Erns protein and a deletion of amino acid at amino acid position 171 of Erns protein. Preferably such attenuated CSFV has one or more mutations within the 6B8 epitope of the E2 protein as disclosed herein.

In one aspect, the attenuated CSFV is derived from the field strain GD18 and contains a deletion of amino acid at amino acid position 79 of Erns protein, a deletion of amino acid at amino acid position 171 of Erns protein, and a deletion of the Npro protein except for the first four amino terminal amino acids. Preferably such attenuated CSFV has one or more mutations within the 6B8 epitope of the E2 protein as disclosed herein.

The following clauses are also described herein and part of disclosure of the invention:

1. A recombinant CSFV (classical swine fever virus) comprising at least one mutation within the 6B8 epitope of the E2 protein, wherein the unmodified 6B8 epitope is specifically recognized by the 6B8 monoclonal antibody.

2. The recombinant CSFV according to clause 1, wherein the at least one mutation within the 6B8 epitope of the E2 protein leads to a specific inhibition of the binding of a 6B8 monoclonal antibody to such mutated 6B8 epitope.

3. The recombinant CSFV according to clause 1 or 2, wherein the 6B8 monoclonal antibody

i) is produced by a hybridoma deposited at CCTCC under the accession number CCTCC C2018120, or

ii) comprises a heavy chain variable region (V_{H}) having an amino acid sequence as set forth in SEQ ID NO: 9 and a light chain variable region (V_{L}) having an amino acid sequence as set forth in SEQ ID NO: 10, or

iii) comprises the CDRs of the monoclonal antibody produced by a hybridoma deposited at CCTCC under the accession number CCTCC C2018120, or

iv) comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

4. The recombinant CSFV according to any one of clauses 1 to 3, wherein the 6B8 epitope of the E2 protein specifically recognized by the 6B8 monoclonal antibody is defined at least by the amino acid residue at position 14, position 22, position 24 and/or positions 24/25 of the E2 protein.

5. The recombinant CSFV according to any one of clauses 1 to 3, wherein the 6B8 epitope of the E2 protein specifically recognized by the 6B8 monoclonal antibody is defined at least by the amino acid residue S14, G22, E24, and/or E24/G25 of the E2 protein, or is defined at least by the amino acid residue S14, G22, G24, and/or G24/G25 of the E2 protein.

6. The recombinant CSFV according to any one of clauses 1 to 3, wherein the 6B8 epitope of the E2 protein specifically recognized by the 6B8 monoclonal antibody is defined at least by the amino acid sequence STNEIGPLGAEG or STDEIGLLGAGG.

7. The recombinant CSFV according to any one of clauses 1 to 6, which comprises a substitution at amino acid position 24 of the E2 protein, a substitution at amino acid positions 24/25 of the E2 protein, a substitution at amino acid position 14 of the E2 protein, and/or a substitution at amino acid position 22 of the E2 protein.

8. The recombinant CSFV according to any one of clauses 1 to 7, in which the amino acid at position 24 of the E2 protein is substituted to R or K, the amino acid at positions 24/25 of the E2 protein is substituted to R/D or K/D, the amino acid at position 14 of the E2 protein is substituted to K, Q or R, and/or the amino acid at position 22 of the E2 protein is substituted to A, R, Q or E, with A and R being preferred.

9. The recombinant CSFV according to any one of clauses 1 to 8, which comprises a substitution of E or G to R or K at amino acid position 24 of the E2 protein, a substitution of E or G to R or K at amino acid position 24 and a substitution of G to D at amino acid position 25 of the E2 protein, a substitution of S to K, Q or R at amino acid position 14 of the E2 protein, and/or a substitution of G to A, R, Q or E, with A and R being preferred, at amino acid position 22 of the E2 protein.

10. The recombinant CSFV according to any one of clauses 1 to 9, wherein the amino acid substitution within the 6B8 epitope of the E2 protein results in a mutated 6B8 epitope sequence of anyone of SEQ ID Nos: 13-14, 31-34.

11. The recombinant CSFV according to any one of clauses 1 to 10, wherein the recombinant CSFV is attenuated.

12. The recombinant CSFV according to any one of clauses 1 to 11, wherein the recombinant CSFV has at least one mutation in in the Erns protein and/or at least one mutation in the Npro protein; preferably such mutation in the Erns protein is a deletion of amino acid at amino acid position 79 of Erns protein and/or a deletion of amino acid at amino acid position 171 of Erns protein, and the mutation in Npro protein is a deletion of the Npro protein except for the first four amino terminal amino acids.

13. The recombinant CSFV according to any one of clauses 1 to 12, wherein the recombinant CSFV is derived from C-strain or a field strain QZ07, GD191, or GD18.

14. The recombinant CSFV according to any one of clauses 1 to 13, wherein the recombinant CSFV is derived from a field strain QZ07, and

(i) comprises a deletion of amino acid at amino acid position 79 of Erns protein, and

(ii) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein.

15. The recombinant CSFV according to any one of clauses 1 to 13, wherein the recombinant CSFV is derived from a field strain QZ07, and

(i) comprises a deletion of amino acid at amino acid position 79 of Erns protein, a deletion of amino acid at amino acid position 171 of Erns protein,

(ii) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein.

16. The recombinant CSFV according to any one of clauses 1 to 13, wherein the recombinant CSFV is derived from a field strain GD18, and

(i) comprises a deletion of amino acid at amino acid position 79 of Erns protein, a deletion of the Npro protein except for the first four amino terminal amino acids, and

(ii) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein.

17. The recombinant CSFV according to any one of clauses 1 to 13, wherein the recombinant CSFV is derived from a field strain GD18, and

(i) comprises a deletion of amino acid at amino acid position 79 of Erns protein, a deletion of amino acid at amino acid position 171 of Erns protein, and

(ii) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein.

18. An isolate nucleic acid coding for a recombinant CSFV according to any one of clauses 1 to 17.

19. A vector comprising the nucleic acid of clause 18.

20. An immunogenic composition comprising the recombinant CSFV according to any one of claims 1 to 16, the isolate nucleic acid coding for a recombinant CSFV according to clause 17, or the vector according to clause 19.

21. The immunogenic composition according to clause 20, wherein said immunogenic composition is a marker vaccine or a DIVA (differentiation between infected and vaccinated animals) vaccine.

22. An immunogenic composition according to clause 20 or 21 for use in a method of preventing and/or treating diseases associated with CSFV in an animal, the method comprising the step of administering the immunogenic composition according to clause 20 or 21 to an animal.

23. The immunogenic composition according to clause 20 or 21 for use in a method of preventing and/or treating diseases associated with CSFV in an animal according to clause 20 or 21, wherein said animal is swine.

24. The immunogenic composition according to clause 20 or 21 for use in a method of preventing and/or treating diseases associated with CSFV in an animal according to clause 20 or 21, wherein said animal is a piglet.

25. The immunogenic composition according to clause 20 or 21 for use in a method of preventing and/or treating diseases associated with CSFV in an animal according to clause 20 or 21, wherein said animal is a piglet of 1 to 4 weeks of age.

26. The immunogenic composition according to clause 20 or 21 for use in a method of preventing and/or treating diseases associated with CSFV in an animal according to clause 20 or 21, wherein said animal is a sow.

27. The immunogenic composition according to clause 20 or 21 for use in a method of preventing and/or treating diseases associated with CSFV in an animal according to clause 20 or 21, wherein said animal is a pregnant sow.

28. The immunogenic composition according to clause 20 or 21 for use in a method of preventing and/or treating diseases associated with CSFV in an animal according to clause 20 or 21, wherein said immunogenic composition is administered only once.

29. The immunogenic composition according to clause 20 or 21 for use in a method of preventing and/or treating diseases associated with CSFV in an animal according to clause 20 or 21, wherein said immunogenic composition is administered only once to the animal and effective in preventing and/or treating diseases associated with CSFV after said single administration of the immunogenic composition.

30. The immunogenic composition according to clause 20 or 21 for use in a method of preventing and/or treating diseases associated with CSFV in an animal according to clause 20 or 21, wherein said immunogenic composition is administered one or several times.

31. The immunogenic composition according to clause 20 or 21 for use in a method of preventing and/or treating diseases associated with CSFV in an animal according to clause 20 or 21, wherein said immunogenic composition is administered one or several times to the animal and effective in preventing and/or treating diseases associated with CSFV after said single or multiple administration of the immunogenic composition.

32. A method of preventing and/or treating diseases associated with CSFV in an animal, the method comprising the step of administering the immunogenic composition according to clause 20 or 21 to an animal in need thereof.

33. A method of marking a CSFV vaccine comprising introducing into a CSFV at least one mutation within the 6B8 epitope of the E2 protein specifically recognized by the 6B8 monoclonal antibody.

34. The method of clause 33 wherein the 6B8 monoclonal antibody

i) is produced by a hybridoma deposited at CCTCC under the accession number CCTCC C2018120, or

ii) comprises a heavy chain variable region (V_{H}) having an amino acid sequence as set forth in SEQ ID NO: 9 and a light chain variable region (V_{L}) having an amino acid sequence as set forth in SEQ ID NO: 10, or

iii) comprises the CDRs of the monoclonal antibody produced by a hybridoma deposited at CCTCC under the accession number CCTCC C2018120, or

iv) comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

35. The method according to clause 33 or 34, wherein the 6B8 epitope of the E2 protein specifically recognized by the 6B8 monoclonal antibody is defined at least by the amino acid residue at position 14, position 22, position 24 and/or positions 24/25 of the E2 protein, for example, the 6B8 epitope of the E2 protein specifically recognized by the 6B8 monoclonal antibody is defined at least by the amino acid residue S14, G22, E24, and/or E24/G25 of the E2 protein, or is defined at least by the amino acid residue S14, G22, G24, and/or G24/G25 of the E2 protein.

36. The method according to clause 33 or 34, wherein the 6B8 epitope of the E2 protein specifically recognized by the 6B8 monoclonal antibody is defined at least by the amino acid sequence STNEIGPLGAEG or STDEIGLLGAGG.

37. The method according to clause 33 or 34, wherein said mutation is a substitution at amino acid position 24 of the E2 protein, a substitution at amino acid positions 24/25 of the E2 protein, a substitution at amino acid position 14 of the E2 protein and/or a substitution at amino acid position 22 of the E2 protein.

38. The method according to clause 33 or 34, wherein the amino acid at position 24 of the E2 protein is substituted to R or K, the amino acid at position 24 of the E2 protein is substituted to R or K and the amino acid at position 25 of the E2 protein is substituted to D respectively, the amino acid at position 14 of the E2 protein is substituted to K, Q or R, and/or the amino acid at position 22 of the E2 protein is substituted to A, R, Q or E, with A and R being preferred.

39. The method according to clause 33 or 34, wherein said mutation is a substitution of E or G to R or K at amino acid position 24 of the E2 protein, a substitution of E or G to R or K at amino acid position 24 and a substitution of G to D at amino acid position 25 of the E2 protein, a substitution of S to K, Q or R at amino acid position 14 of the E2 protein and/or a substitution of G to A, R, Q or E, with A and R being preferred, at amino acid position 22 of the E2 protein.

40. The method according to clause 33 or 34, wherein the mutation within the 6B8 epitope of the E2 protein results in a mutated 6B8 epitope sequence of any one of SEQ ID Nos: 13-14, 31-34.

41. The method according to clause 33 or 34, wherein the CSFV vaccine is an attenuated vaccine.

42. The method according to clause 33 or 34, wherein the CSFV is derived from C-strain or a field strain QZ07 or GD18.

43. A method of differentiating animals infected with CSFV from animals vaccinated with the immunogenic composition of any one of clause 20 or 21, comprising

a) obtaining a sample, and

b) testing said sample in an immuno test.

44. The method according to clause 43, wherein the immuno test comprises testing whether an antibody specifically recognizing the 6B8 epitope of the CSFV E2 protein can bind to the CSFV E2 protein in the sample.

45. The method according to clause 43, wherein the antibody specifically recognizing the 6B8 epitope

i) is produced by a hybridoma deposited at CCTCC under the accession number CCTCC C2018120, or

ii) comprises a heavy chain variable region (V_{H}) having an amino acid sequence as set forth in SEQ ID NO: 9 and a light chain variable region (V_{L}) having an amino acid sequence as set forth in SEQ ID NO: 10, or

iii) comprises the CDRs of the monoclonal antibody produced by a hybridoma deposited at CCTCC under the accession number CCTCC C2018120, or

iv) comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

46. The method according to clause 43, wherein the immuno test comprises testing whether an antibody specifically recognizing a 6B8 epitope of the CSFV E2 protein is present in the sample, and/or testing whether an antibody specifically recognizing a mutated 6B8 epitope of the CSFV E2 protein is present in the sample.

47. The method according to any one of clauses 43 to 46, wherein the immuno test is an EIA (enzyme immunoassay) or ELISA (enzyme linked immunosorbent assay), preferably a double competitive ELISA.

48. An antibody or an antigen-binding fragment thereof, wherein said antibody is produced by a hybridoma deposited at CCTCC under the accession number CCTCC C2018120, or wherein said antibody comprises a heavy chain variable region (V_{H}) having an amino acid sequence as set forth in SEQ ID NO: 9 and a light chain variable region (V_{L}) having an amino acid sequence as set forth in SEQ ID NO: 10, or wherein the antibody comprises the CDRs of the monoclonal antibody produced by a hybridoma deposited at CCTCC under the accession number CCTCC C2018120, or wherein the antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

49. A kit for differentiating animals infected with CSFV from animals vaccinated with the immunogenic composition of any one of clause 20 or 21, which comprises the antibody of claim 39, or an antigen-binding fragment thereof.

50. An recombinant attenuated CSFV, wherein the recombinant attenuated CSFV has at least one mutation in the Erns protein and/or at least one mutation in the Npro protein; preferably such mutation in the Erns protein is a deletion of amino acid at amino acid position 79 of Erns protein and/or a deletion of amino acid at amino acid position 171 of Erns protein, and the mutation in Npro protein is a deletion of the Npro protein except for the first four amino terminal amino acids.

51. The recombinant attenuated CSFV according to clause 50, wherein the recombinant attenuated CSFV is derived from C-strain or a field strain QZ07 or GD18.

52. The recombinant attenuated CSFV according to clause 51, wherein the recombinant attenuated CSFV is derived from a field strain QZ07, and comprises a deletion of amino acid at amino acid position 79 of Erns protein.

53. The recombinant attenuated CSFV according to clause 51, wherein the recombinant CSFV is derived from a field strain QZ07, and comprises a deletion of amino acid at amino acid position 79 of Erns protein, and a deletion of amino acid at amino acid position 171 of Erns protein.

54. The recombinant attenuated CSFV according to clause 51, wherein the recombinant CSFV is derived from a field strain GD18, and comprises a deletion of amino acid at amino acid position 79 of Erns protein, and a deletion of the Npro protein except for the first four amino terminal amino acids.

55. The recombinant attenuated CSFV according to clause 51, wherein the recombinant CSFV is derived from a field strain GD18, and comprises a deletion of amino acid at amino acid position 79 of Erns protein, and a deletion of amino acid at amino acid position 171 of Erns protein.

56. An isolate nucleic acid coding for a recombinant attenuated CSFV according to any one of clauses 51 to 55.

57. A vector comprising the nucleic acid of clause 56.

58. An immunogenic composition comprising the recombinant attenuated CSFV according to any one of clauses 50 to 55, the isolate nucleic acid coding for a recombinant attenuated CSFV according to clause 56, or the vector according to clause 57.

59. An immunogenic composition according to clause 58 for use in a method of preventing and/or treating diseases associated with CSFV in an animal, the method comprising the step of administering the immunogenic composition according to clause 58 to an animal in need thereof.

60. The immunogenic composition according to clause 58 or 59 for use in a method of preventing and/or treating diseases associated with CSFV in an animal according to clause 58 or 59, wherein said animal is swine.

61. The immunogenic composition according to clause 58 or 59 for use in a method of preventing and/or treating diseases associated with CSFV in an animal according to clause 58 or 59, wherein said animal is a piglet.

62. The immunogenic composition according to clause 58 or 59 for use in a method of preventing and/or treating diseases associated with CSFV in an animal according to clause 58 or 59, wherein said animal is a piglet of 1 to 4 weeks of age.

63. The immunogenic composition according to clause 58 or 59 for use in a method of preventing and/or treating diseases associated with CSFV in an animal according to clause 58 or 59, wherein said animal is a sow.

64. The immunogenic composition according to clause 58 or 59 for use in a method of preventing and/or treating diseases associated with CSFV in an animal according to clause 58 or 59, wherein said animal is a pregnant sow.

65. The immunogenic composition according to clause 58 or 59 for use in a method of preventing and/or treating diseases associated with CSFV in an animal according to clause 58 or 59, wherein said immunogenic composition is administered only once.

66. The immunogenic composition according to clause 58 or 59 for use in a method of preventing and/or treating diseases associated with CSFV in an animal according to clause 58 or 59, wherein said immunogenic composition is administered only once to the animal and effective in preventing and/or treating diseases associated with CSFV after said single administration of the immunogenic composition.

67. The immunogenic composition according to clause 58 or 59 for use in a method of preventing and/or treating diseases associated with CSFV in an animal according to clause 58 or 59, wherein said immunogenic composition is administered one or several times.

68. The immunogenic composition according to clause 58 or 59 for use in a method of preventing and/or treating diseases associated with CSFV in an animal according to clause 58 or 59, wherein said immunogenic composition is administered one or several times to the animal and effective in preventing and/or treating diseases associated with CSFV after said single or multiple administration of the immunogenic composition.

69. A method of preventing and/or treating diseases associated with CSFV in an animal, the method comprising the step of administering the immunogenic composition according to clause 58 to an animal in need thereof.

70. A method of preventing and/or treating diseases associated with CSFV in an animal, the method comprising the step of administering the immunogenic composition according to clause 59 to an animal in need thereof.

71. A method of making a recombinant attenuated CSFV vaccine comprising introducing into a CSFV at least one mutation in the Erns protein and/or at least one mutation in the Npro protein; preferably such mutation in the Erns protein is a deletion of amino acid at amino acid position 79 of Erns protein and/or a deletion of amino acid at amino acid position 171 of Erns protein, and the mutation in Npro protein is a deletion of the Npro protein except for the first four amino terminal amino acids.

72. The method according to clause 71, wherein the recombinant attenuated CSFV is derived from C-strain or a field strain QZ07 or GD18.

73. The method according to clause 72, wherein the recombinant attenuated CSFV is derived from QZ07, and wherein amino acid at amino acid position 79 of Erns protein is deleted.

74. The method according to clause 72, wherein the recombinant attenuated CSFV is derived from QZ07, and wherein the amino acids at amino acid position 79 of and 171 of Erns protein are deleted.

75. The method according to clause 72, wherein the recombinant CSFV is derived from a field strain GD18, and wherein amino acid at amino acid position 79 of Erns protein, the amino acids of the Npro protein except for the first four amino terminal amino acids are deleted.

76. The method according to clause 72, wherein the recombinant CSFV is derived from a field strain GD18, and wherein amino acids at amino acid position 79 and 171 of Erns protein are deleted.

### EXAMPLES

The subsequent examples further illustrate the invention in an exemplified manner. It is understood that the invention is not limited to any of those examples as described below. A person skilled in the art understands that the performance, results and findings of these examples can be adapted and applied in a broader sense in view of the general description of the invention.

### Example 1: CSFV Attenuation by Deletions in Npro and/or Erns

Three field isolates of genogroup 2.1 of CSFV, QZ07, GD18, and GD191, which have been adapted to PK/WRL cell culture and determined as representing strains of low, moderate, and high virulence, were used to construct the infectious clone for GMO attenuation. The whole genome sequences of field isolates QZ07, and GD18 were shown in SEQ ID NO:1, and SEQ ID NO:2, respectively.

In brief, the 5′-end 2 kb fragment of the whole 12 kb genome was first cloned into pACYC and via this platform, deletions (the deletion of Npro gene from position 5 to 168 of the Npro protein, and an amino acid (Histidine) deletion in Erns protein at position 79) were performed. Then, two DNA segments covering the other portion of the whole 12 kb genome (2 kb position to the 3′ end of the genome) were amplified from cDNA of each field isolate and inserted into BAC plasmid pBeloBAC. The mutated first 2 kb of CSFV genome was inserted into the pBeloBAC with the two more segments to assemble the CSFV infectious clone via RED recombination method. The resulted infectious clones were named as GD18-ddNpro-ErnsH, GD191-ddNpro-ErnsH, QZ07-sdErnsH and QZ07-ddNpro-ErnsH. QZ07-sdErnsH only comprises the single Histidine deletion at amino acid position 79 of the Ems protein. Live viruses with the deletions were rescued from the infectious clones of the three field isolates.

After 10 passages of the rescued live virus, the structural sequences of the four P10 viruses were amplified and sequenced. Specifically, viral RNA was extracted from p10 virus stock using QIAamp viral kit, and then the structural genes (1-4 kb) were amplified using SuperScript III Platinum One Step RT-PCR kit. The amplified DNA was then purified, sequenced and compared to the parental virus sequence (wild type) after alignment. Sequencing results revealed that the deletions are stable after 10 passages.

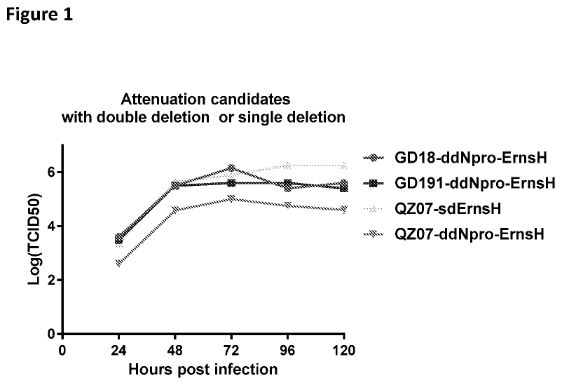

To find out the peak titer of each passaged virus, the growth curve of each virus was plotted by sampling at every 24 hours after infection at MOI of 0.01. Specifically, T25 flasks with PK/WRL cells were infected with each P10 stock virus at MOI of 0.001. The infection was done with corresponding amount of virus diluted in 1 ml MEM infection medium. After 1 hour incubation, the infection medium was discarded and the flask was replenished with 5 ml of fresh medium. 200 μl of sample was taken at every 24 hours interval up to 120 hours after infection. The samples were then titrated to plot the growth curve as shown in FIG. 1. The data suggested that the peak titer meets the desired criteria after 10 passages.

Solely for a strict safety consideration (avoiding back mutation), G0191, which is highly virulent, was excluded from further analysis, although it is also a promising candidate. In addition, the deletion in Npro was excluded from QZ07. And a further amino acid (Cysteine) deletion in Erns protein at position 171 was introduced to either GD18 or QZ07 to increase the safety profile, for example for use in pregnant sows.

Finally, four candidates construed with mutations for attenuation: QZ07-sdErnsH, QZ07-ddErnsHC, GD18-ddNproErnsH, and GD18-ddErnsHC.

TABLE 1
Attenuation Candidates

| | Parental | | Deletion | | |
|---|---|---|---|---|---|
| Parental Strain | Virulence | Candidates* | N^{pro} | E^{ms}-His (79) | E^{ms}-Cys(171) |
| GD18 | Moderate | GD18-ddNproErnsH | ✓ | ✓ | No |
| | | GD18-ddErnsHC | No | ✓ | ✓ |
| QZ07 | Low | QZ07-sdErnsH | No | ✓ | No |
| | | QZ07-ddErnsHC | No | ✓ | ✓ |

sd, single deletion; dd, double deletion.

### Example 2: Identification and Incorporation of DIVA Sites

A core feature of the desired new vaccine is its ability to differentiate vaccinated animal from infected animal (DIVA). The DiVA feature will be an essential improvement from the traditional lapnized C strain and has important technical advantage. The strategy of introducing DIVA feature is to alter one or more critical epitope in the immune dominant E2 protein surface and use ELISA to demonstrate the absence of antibody recognizing wild type epitope as an indication of vaccination (negative DIVA).

To implement this strategy, the inventors chose a strongly neutralizing mouse mAb 6B8. Hybridomas producing monoclonal antibody 6B8 was obtained from Zhejiang University and deposited under the accession number CCTCC C2018120 at CCTCC (CHINA CENTER FOR TYPE CULTURE COLLECTION), Wuhan University, Wuhan 430072, P.R. China) on Jun. 13, 2018. Sequencing of the monoclonal antibody 6B8 revealed that it has a heavy chain variable region (V_{H}) having an amino acid sequence as set forth in SEQ ID NO: 9 and a light chain variable region (V_{L}) having an amino acid sequence as set forth in SEQ ID NO: 10. CDRs of this antibody can be easily determined by various methods known in the art, such as Kabat method. For example, mAb 6B8 comprises a VH CDR1 of the amino acid sequence set forth in SEQ ID NO:25, a VH CDR2 of the amino acid sequence set forth in SEQ ID NO:26, a VH CDR3 of the amino acid sequence set forth in SEQ ID NO:27, a VL CDR1 of the amino acid sequence set forth in SEQ ID NO:28, a VL CDR2 of the amino acid sequence set forth in SEQ ID NO:29, and a VL CDR3 of the amino acid sequence set forth in SEQ ID NO:30.

### 1. Characterization of 6B8 mAb

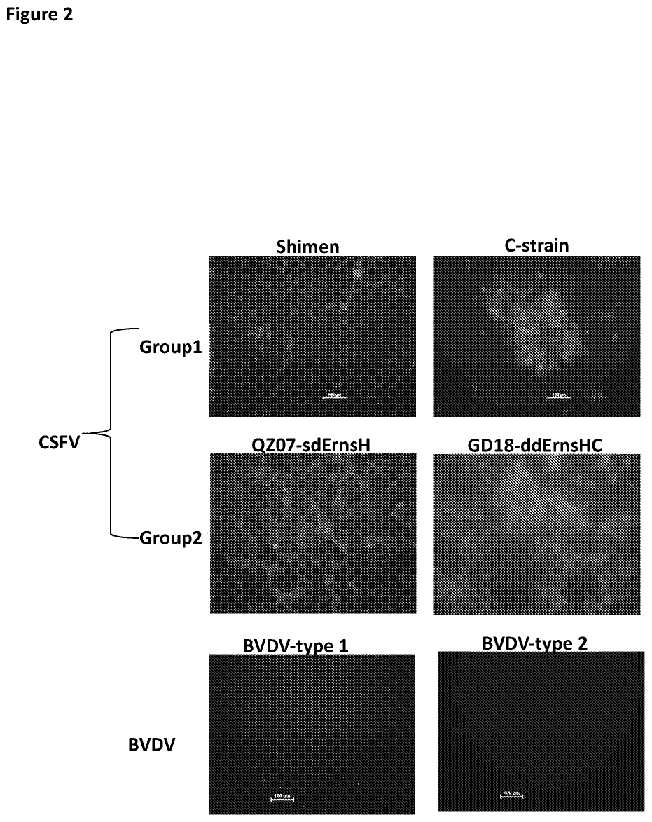

To investigate whether mAb 6B8 can be used for most CSFVs, the inventors tested the binding of mAb 6B8 with various CSF viruses, such as CSFVs from Group 1 (including Shimen strain and C-strain) and from Group2 (including QZ07 and GD18), with two BVDVs as control. The results were shown in FIG. 2. Additional 8 field CSFV isolates from genotype group 2 were also tested as positive for 6B8 mAb (data not shown). These data indicated that 6B8 recognizes a conserved epitope presents on most of CSF viruses, while has no reaction with BVDV viruses.

### 2. Identification of Critical Amino Acids for 6B8 Binding

After serial passage of C-strain virus in PK/WRL cell cultures in the presence of mAb 6B8, escape mutants emerged and can grow in the presence neutralizing concentration of 6B8 antibody. Four clones of such escape mutants were obtained and they all escaped 6B8 binding. Their E2 genes were sequenced and the sequencing results indicated that two nucleotide mutation in two codons (GGAGGT to AGAGAT). These changes translated to two amino acid mutations at consecutive positions 24&25 (Gly-Gly to Arg-Asp, or GG to RD).

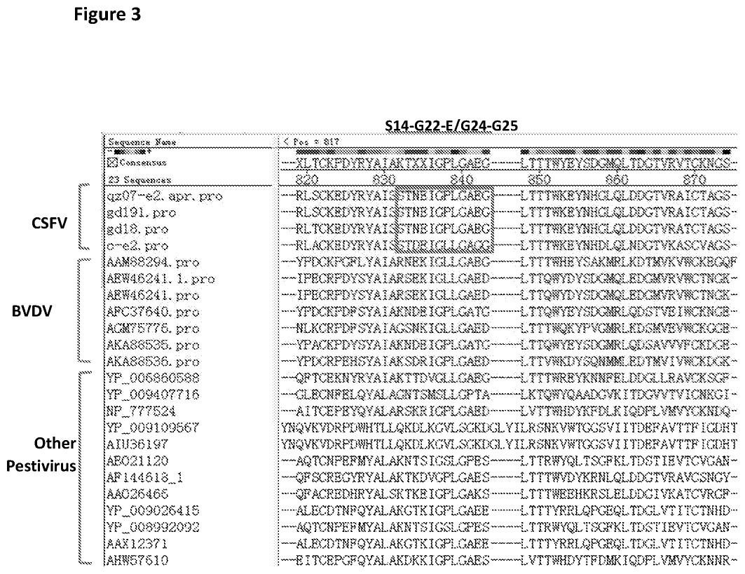

Then, E2 sequence alignment (QZ07, GD18, GD191 and C-strain) was performed with BVDV and other pestivirus E2 to identify other potential critical amino acid for 6B8 binding (FIG. 3). By this approach, additional potential critical amino acids were identified, such as amino acids at position 14 and position 22.

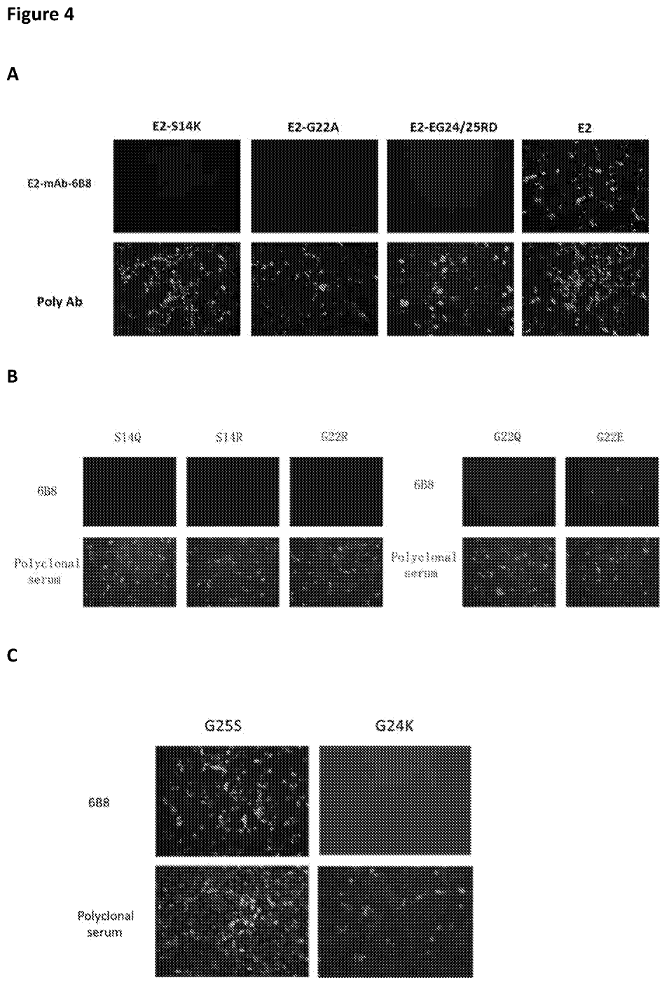

All these potential mutations (S14K, G22A, E24R/G25D) were introduced into E2 expression vector individually to test its effect on 6B8 binding. E2 gene was cloned into pCI-neo-Tag vector (Promega, cat #E1841) to generate expression vectors. After confirmation of the correct expression of E2 protein, all the mutations were introduced into the E2 expression vector. These vectors were then transfected into PK/WRL cells using Lipofectamine3000 (Invitrogen, cat #L3000015) in 24-well plate. 24 hours post transfection, the cells were fixed with 4% formaldehyde and then treated with 0.1% Triton X-100. Cell are then stained with mAb 6B8 or a rabbit-polyclonal antibody against CSFV (used as positive control to detect CSFV with modified 6B8 epitopes), and corresponding Alexa Fluor®488 conjugated second antibody (Invitrogen cat #21206) in an IFA (immunoinfluoscent assay) test. As shown in FIG. 4 A, microscopic examination revealed that 514K, G22A, E24R/G25D mutations are critical for abolishing 6B8 binding. The inventors also tested the effect of other mutations at positions 14, 22, 24 and 25 individually on the binding with 6B8 antibody. As shown in FIG. 4 B, mutations S14Q, S14R, and G22R totally abolished the binding of 6B8 while G22E, G22Q partially affect the binding of 6B8, further indicating that positions 14 and 22 are critical for 6B8 binding. As shown in FIG. 4 C, a single mutation G24K (for C strain) totally abolished the binding of 6B8, also supporting that position 24 is critical for 6B8 binding. G25S alone cannot abolish the binding of 6B8. However, as the position 25 Gly to Asp mutation emerged together with the mutation at position 24, and thus the two mutations can be considered as one mutation (24/25 mutation).

The results suggest that mutations at position 14, 22, 24 and/or 24/25 may be used for DIVA. The results also suggest that the mutation of 6B8 epitope does not substantially alter the overall immunogenicity of the E2 protein, as the mutated E2 protein can still be recognized by polyclonal antibody against CSFV.

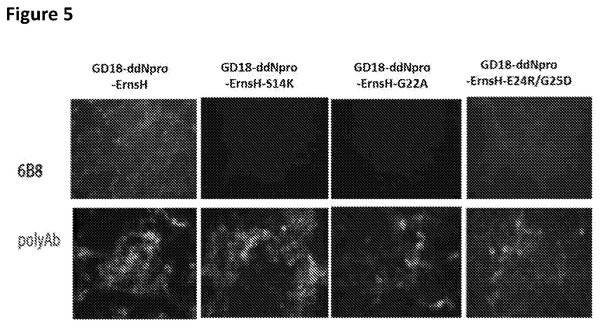

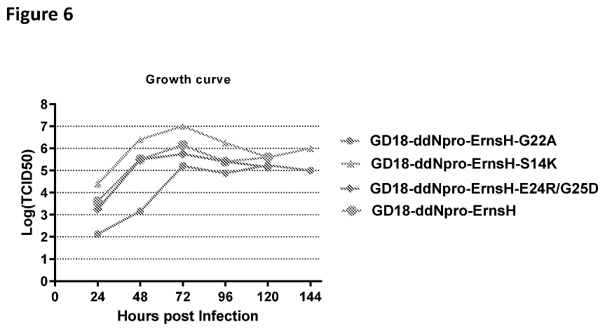

These 6B8 epitope mutations were then introduced into infectious clone GD18-ddNpro-ErnsH, resulted in 3 infectious clones (GD18-ddNpro-ErnsH-S14K, GD18-ddNpro-ErnsH-G22A, GD18-ddNpro-ErnsH-EG24/25RD). Live viruses were rescued from these 3 infectious clones and IFA confirmed their binding properties to mAb 6B8 or polyclonal serum antibody (FIG. 5). Characterization of these viruses indicated that these mutations are stable over 10 passages and the peak titer of each virus is similar to that of the parental GD18-ddNpro-ErnsH virus (FIG. 6). As a result, mutations S14K, G22A, E24R/G25D were appropriate for DIVA development.

### 3. Constructing DIVA Candidate Viruses

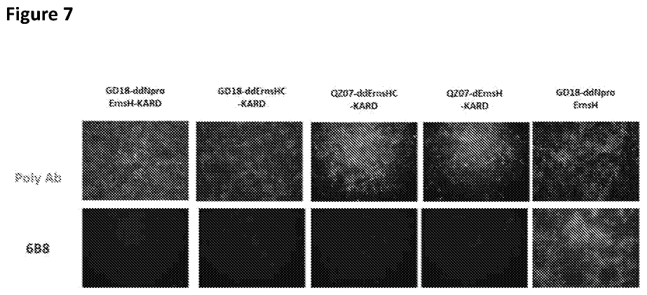

To increase the chance of success, a combination of the potential DIVA mutations were introduced into the four double deletion or single deletion infectious clones in Example 1 and resulted in four final DIVA infectious clones: QZ07-sdErnsH-KARD, QZ07-ddErnsHC-KARD, GD18-ddNproErnsH-KARD, and GD18-ddErnsHC-KARD (KARD means S14K, G22A, and E24R/G25D mutations). Lives viruses were rescued from these four infectious clones, passaged and characterized via IFA (FIG. 7). The results indicated that the candidates containing four amino acids mutation KARD are infectious in vitro and do not reaction with 6B8 mAb. Other combinations of DIVA mutations, such as RD (E24R_/G2SD), KRD (S14K, and E24R/G25D) were also introduced into the four double deletion or single deletion infectious clones in Example 1 and had similar results.

### Example 3: Safety Validation of Four Candidate Recombinant Viruses

The objective of this Example was to evaluate the safety of four CSFV attenuated viruses with DIVA mutations, namely QZ07-sdErnsH-KARD, QZ07-ddErnsHC-KRD, GD18-ddNproErnsH-KRD, and GD18-ddErnsHC-KARD in 3-week-old piglets.

A total of 29 piglets were assigned into 5 groups. 5 piglets were used as negative controls (Group 5), whereas other 24 piglets were randomly divided into four groups (Groups 1, 2, 3 and 4), 6 piglets per group. On Day 0, animals in groups 1, 2, 3 and 4 were inoculated into left neck with 1 mL (5 logs TCID₅₀/mL) per piglet of the QZ07-sdErnsH-KARD, QZ07-ddErnsHC-KRD, GD18-ddNproErnsH-KRD, and GD18-ddErnsHC-KARD, respectively, as shown on Table 2. All piglets were clinical healthy and free for CSFV and PRRSV antibodies and free of antigen including BVDV, PRV on Day 0. All animals were healthy at the time of inoculation.

TABLE 2
Study design

| | No. of | | | Dosing | |
|---|---|---|---|---|---|
| Group | Animals | Inoculation | Dose on Day0 | Route | Necropsy |
| 1 | 6 | GD18-ddNproErnsH-KRD | 1 mL 5 Logs TCID₅₀/mL | IM | On D21 |
| 2 | 6 | QZ07-sdErnsH-KARD | 1 mL 5 Logs TCID₅₀/mL | IM | On D21 |
| 3 | 6 | GD18-ddErnsHC-KARD | 1 mL 5 Logs TCID₅₀/mL | IM | On D21 |
| 4 | 6 | QZ07-ddErnsHC-KRD | 1 mL 5 Logs TCID₅₀/mL | IM | On D21 |
| 5 | 5 | N/A | N/A | IM | On D21 |

### Study Procedure

### Body Temperature

Body temperature was measured for all animals twice daily from D-4 to D21. Body temperatures were recorded in Celsius unit. The average body temperature of four days before DO and prior to vaccination on DO (total nine measure points) was set as normal temperature for each piglet. The below description of temperature was considered as normal: a) temperature arise within 0.5° C.; b) temperature arise 0.5-1° C. for no more than 4 consecutive time points; c) temperature arise 1-1.5° C. for one animal for no more than 2 consecutive time points. The body temperature on DO was measured prior to administration of the IVP (Investigational Veterinary Products, namely the recombinant virus to be tested in a specific amount) and 4 hours after administration of the IVPs. The body temperature on D21 was only measured once prior to necropsy.

### Clinical Observation

Clinical observation was carried out on all animals twice daily from D4 to D21. Clinical observations consist of assessments of liveliness, appetite and other abnormalities by using the score system as shown in Table 3. A zero indicates no clinical signs, and increased clinical score indicates an increasing degree of severity of clinical signs. If individual animal shows clinical score above 1 with 3 consecutive observation points, it is to be considered as CSF related clinical sign. The clinical observation on DO was carried out prior to administration of the IVP and 4 hours after administration of the IVP. The clinical observation on D21 was only carried out once prior to necropsy.

TABLE 3
Clinical Score Instruction

| No. | Parameter | Criteria | Score |
|---|---|---|---|
| 1 | Liveliness | Attentive (curious, stands up immediately) | 0 |
| | | Slightly reduced (stands up hesitantly, but | 1 |
| | | without help) | |
| | | Tired, gets up only when forced to, lies | 2 |
| | | down again | |
| | | Dormant, will not stand up | 3 |
| 2 | Appetite | Greed, hungry | 0 |
| | | Eats slowly when fed | 1 |
| | | Does not eat when fed, but taste food | 2 |
| | | Does not eat at all, shows no interest | 3 |
| | | for food | |
| 3 | Other | Normal | 0 |
| | abnor- | Thin | 1 |
| | malities | Diarrhea | 1 |
| | | Highly inflammated conjunctive, turbid | 1 |
| | | secretion | |

### Injection Site Reaction

Injection site reaction was evaluated for all animals twice daily from DO to D21. Injection site reactions consist of assessments of redness, swelling, heat, and pain by using the score system as shown in Table 4. A zero indicates no injection site reaction, and increased score indicate an increasing degree of injection site reaction. If total score of injection site reaction above 1 with 3 consecutive observation points, it is to be considered as vaccine related injection site reaction. The Injection site reaction on DO was carried out prior to administration of the IVP and 4 hours after administration of the VP. The Injection site reaction on D21 was only carried out once prior to necropsy.

TABLE 4
Scoring system for injection site reaction

| No. | Parameter | Criteria | Score |
|---|---|---|---|
| 1 | Redness | None (no observable redness) | 0 |
| | | Slight (reddening og injection site and | 1 |
| | | immediate surrounding area) | |
| | | Moderate (reddening of the injection site and | 2 |
| | | large surrounding area) | |
| | | Severe (dark red or black coloration of the | 3 |
| | | injection site and surrounding area) | |
| 2 | Swelling | None (no swelling perceptible) | 0 |
| | | Minimal (only palpable swelling at the | 1 |
| | | injection site) | |
| | | Slight (slight swelling at the injection | 2 |
| | | site and immediate) | |
| | | Moderate (distinct swelling of the injection | 3 |
| | | site and surrounding site) | |
| | | Sever (marked swelling extending far from the | 4 |
| | | injection site) | |
| 3 | Heat | None (skin temperature similar to skin over | 0 |
| | | other body areas by feel) | |
| | | Warm (skin temperature slightly warmer than | 1 |
| | | skin of other body areas by feel) | |
| | | Hot (skin temperature markedly warmer than | 2 |
| | | skin of other body areas by feel) | |
| 4 | Pain | Absent (no perceptible signs of pain during | 0 |
| | | palpation) | |
| | | Present (perceptible signs of pain during | 1 |
| | | palpation) | |

### Leukocytes Count

Approximately 1 mL blood samples were collected from each piglet on DO, 3, 5, 7, 10 and 21. Sample was collected using suitable needles and syringes and transferred to EDTA coated tubes. Leucocytes from blood samples were analyzed immediately by Exigo-61812 automatic blood instrument. Each sample was tested twice.

### Necropsy

All surviving piglets were humanely terminated and necropsied on D21. The main target tissues/organs, e.g. tonsil, kidney, spleen, lymph nodes, ileum/rectum, etc. were examined for CSF related pathogenicity. Clinical sign related CSF associated gross lesion was fixed in formalin for possible future evaluation (only Group 1-4). The fresh tonsil was collected from each piglet for CSFV virus isolation.

Spleen infarction, ulceration of intestine mucosa in rectum and ileum, haemorrhage in kidneys, lymph nodes, sub cutis petechial and mucosa of urinary bladder are of gross pathological findings of CSFV infection. A pig with 3 aforementioned findings was considered infected by CSFV. The absence of the aforementioned findings was considered not infected by CSFV. The animals with 1 or 2 aforementioned findings were considered positively infected by CSFV if its CSFV virus isolated positive.

### Virus Isolation

Nasal swabs were collected from each piglet on DO, 3, 5, 7, 10 and 21. A sterilized swab was inserted into a nasal cavity, gently rotated to collect the secretions, and sample procedure was repeated for another nasal cavity. After collection, two nasal swabs were pooled into a sterilized tube with storage buffer (4 mL MEM containing 10% FBS and 1% antibiotic). Each nasal swab sample was aliquot into 3 tubes, 600 μl/tube. All samples for Virus isolation were stored below −40° C. for CSFV virus isolation.

For tonsil homogenization, tonsil tissues were cut into small pieces of 0.4-0.5 g, and were ground in a small amount of cell culture medium into a homogeneous paste using a mortar and pestle. Alternatively, an appropriate crushing machine or automatic homogenizer was used, and 10% (w/v) suspension was made by adding Hanks' minimal essential medium (MEM), followed by freeze-thaw cycle for 3 times to lyse cells. Finally, the samples were centrifuged at 4° C., at the speed of 9000 g for 15 minutes, and clarified homogenates were filtrated using 0.45 μm filters.

For nasal swab samples, swab in viral transport medium (VTM) was removed from the VTM. The frozen swab samples shall be thawed at first. The swab was gently vortexed or swirled in the fluid and reamed against the side of the tube. The suspensions was transferred into a new centrifuge tube, centrifuged at 4° C. at a speed of 8,000×g for 10 min, and the supernatant was taken for further inoculation. Sterile filtration were performed by using syringe filters (0.45 μm or/and 0.22 μm) if samples were contaminated.

24-hours before the scheduled assay, 48-well flat bottom micro-plates were planted. The PK/WRL cells were planted at a concentration of 0.7-1.0×10⁵/mL with 500 μL per well. After one day of incubation, culture media in the well were discarded with pipette, leaving only residual amount of MEM to prevent the cell monolayer from drying out. 200 μL of test samples and positive control were immediately inoculated onto a PK/WRL monolayer in duplicate, and allowed for adsorption for 1-2 hours at 37° C. The inoculum was discarded with pipette and 500 μL per well of fresh growth media with 50× Pen Strep (Life-tech, Cat #15140-122) and 250× GENTAMICIN/AMPHOTERICIN (Invitrogen, Cat #R01510) were dispensed into the wells. The plates were incubated at 37° C., 5% CO₂ incubator for 3 days.

To improve sensitivity, virus isolation was performed over two passages, as described below. The above micro-plates were sealed with tape around the plates, and frozen at −80° C. for at least 1 hour and then thawed at RT twice to lyse the cells. 200 μL of above prepared supernatants were inoculated in duplicate in cell culture 48-well plates. The cells were planted with Pen Strep and GENTAMICIN/AMPHOTERICIN as described above. The cultures were incubated for 3 days at 37° C. in a CO₂ incubator and the passage was repeated once more. The passage 3 plates were analysed by IFA.

### Immunofluorescence (IFA) Methods

The medium of virus-infected and mock wells was aspirated. The wells were washed 2 times with 1×PBS for 5 min per wash, and then fixed by the addition of 4% formaldehyde for 30 min. After fixation, the wells were washed as described above and 0.1% Triton X-100 was added for 15 min permeabilization. Then all wells were washed and a properly diluted primary polyclonal antibody (rabbit serum against CSFV) was added to each well followed by incubation at 37° C. for 1 h. After rinsing with 1×PBS for 3 times, 200-fold diluted anti-rabbit secondary antibodies (e.g. donkey anti-rabbit IgG Alexa Fluor®488 conjugated, Invitrogen cat #21203) were added to each well. Following incubation for 1 h, the wells were washed with 1×PBS for 2 times and finally observed under fluorescence microscopy for IFA positive wells.

### Study Results

### Body Temperature

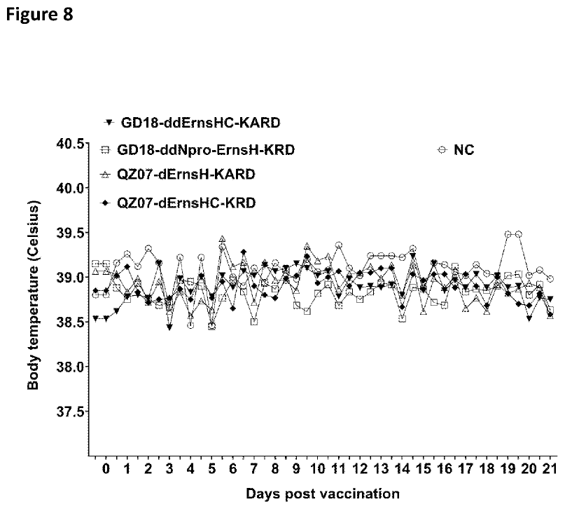

The mean of body temperature of each group were between 37.5-39.5° C. (physiological range) at all measure point which were shown in FIG. 8. According to fever definition, there was no fever detected in all groups after the dosing of the IVPs (Investigational Veterinary Products).

### Clinical Observation

There were no treatment associated clinical signs was observed in all groups after the dosing of the IVPs.

### Injection Site Reaction

There were no abnormal injection site reaction observations in all vaccinated animals after the dosing of the IVPs.

### Gross Pathology

All the piglets were necropsied at the end of the study. No gross lesion was observed in all animals after the dosing of the IVPs.

### Leukocytes Count

Leukocytes remained stable compared with Day 0 throughout the study, and no difference was found between IVPs dosing groups and control group.

### CSFV Isolation in Tonsil

All tonsil samples were VI (virus isolation) negative.

### Nasal Virus Shedding

All nasal swabs were VI negative.

### Conclusion:

The results of safety research were summary in table 5.

TABLE 5
Safety performance summary

| | | | | | Virus in |
|---|---|---|---|---|---|
| | | Clinical | Gross | Shed- | tonsil |
| Group | Fever | signs | pathology | ding | on DPI21 |
| QZ07-sdErnsH-KARD | 0/6 | 0/6 | 0/6 | 0/6 | 0/6 |
| QZ07-ddErnsHC-KRD | 0/6 | 0/6 | 0/6 | 0/6 | 0/6 |
| GD18- | 0/6 | 0/6 | 0/6 | 0/6 | 0/6 |
| ddNrpo-ErnsH-KRD | | | | | |
| GD18-ddErnsHC-KARD | 0/6 | 0/6 | 0/6 | 0/6 | 0/6 |

Safety study showed no fever, clinical signs, leucopenia, or gross pathology in all vaccinated groups. Also, no virus was isolated in tonsil at DPI 21 and there is no shedding of virus to environment. In conclusion, all four recombinant viruses are safe in 3-week-old piglets.

### Example 4: Efficacy Validation of Four Candidate Recombinant Viruses

The objective of this Example was to evaluate the efficacy of the four recombinant viruses, namely QZ07-sdErnsH-KARD, QZ07-ddErnsHC-KRD, GD18-ddNproErnsH-KRD, and GD18-ddErnsHC-KARD in 3-week-old piglets.

A total of 55 piglets were assigned into 6 groups (Groups 1, 2, 3, 4, 5 and 6), ten piglet each in Group 1, 2, 3, 4 were used for recombinant virus test while another 10 piglets in group 5 served as challenge control. The rest five piglets in Group 6 which served as negative control. On Day 0, animals in groups 1, 2, 3 and 4 were inoculated into left neck with 1 mL (3 logs TCID₅₀/mL) per piglet of the QZ07-sdErnsH-KARD, QZ07-ddErnsHC-KRD, GD18-ddNproErnsH-KRD, and GD18-ddErnsHC-KARD, respectively, which as shown on Table 6. Group 5 were inoculated into left neck with 1 mL PBS on Day 0, served as challenge control. Animals in groups 1, 2, 3, 4 and 5 were inoculated into left neck with CSFV Shimen strain at dose ≥10⁵ MLD/mL on 14 days after vaccination. All piglets were clinical healthy and free for CSFV and PRRSV antibodies and free of antigen including BVDV, PRV on Day 0. All animals were healthy at the time of immunization.

TABLE 6
Summary of Study design

| | No. of | | | Challenge on | |
|---|---|---|---|---|---|
| Group | Animals | Inoculation | Treatment on Day0 | Day 14 | Necropsy |
| 1 | 10 | QZ07-sdErnsH-KARD | IM, 1 mL 3 Logs | IM, 1.0 ML CSF | On Day 30 |
| | | | TCID₅₀/mL | Shimen strain | |
| 2 | 10 | QZ07-ddErnsHC-KRD | IM, 1 mL 3 Logs | blood stock | On Day 30 |
| | | | TCID₅₀/mL | (≥10⁵ MLD) | |
| 3 | 10 | GD18-ddNproErnsH-KRD | IM, 1 mL 3 Logs | | On Day 30 |
| | | | TCID₅₀/mL | | |
| 4 | 10 | GD18-ddErnsHC-KARD | IM, 1 mL 3 Logs | | On Day 30 |
| | | | TCID₅₀/mL | | |
| 5 | 10 | PBS | IM, 1 mL | | On Day 30 |
| 6 | 5 | N/A | N/A | N/A | On Day 30 |

Body temperatures, clinical score were collected daily from D12 to D30. On days post challenge (DPC) 0, 3, 5, 7, and 16, whole blood samples of all animals were collected for Leucocyte Count. All serum samples on DO were collected for study valid test. On DPC 16, all survival piglets were humanely terminated and scored for gross pathology findings.

The challenge control and negative control groups were CSFV antibody negative up to the day of challenge (DPC0), and the negative control group remained CSFV antibody negative for the remainder of the study (DPC16), the morbidity and mortality were 100% and 90% in challenge control group, thus validating the study.

### Study Procedure

### Body Temperature

Body temperature was measured for all animals once daily from DPC −2 to DPC 16. Body temperatures were recorded in Celsius. The average body temperature of three days prior to challenge (DPC-2 to DPC 0) was set as normal temperature for each piglet. If individual animals met below criteria is to be considered as fever: 1° C. <Body temperature and 3 (≥) consecutive measure point; 1.5° C. <Body temperature and 2 (≥) consecutive measure point.

### Clinical Observations

Clinical observation was carried out on all animals once daily from DPC 0 to DPC 16. Clinical observation consist of assessments of liveliness, body tension (stiffness, cramps), body shape (body condition, thinned musculature), breathing, walking, skin, appearance of conjunctiva, appetite and defecation as shown in Table 6. A zero indicates no clinical signs, and increased clinical score indicate an increasing degree of severity of clinical signs. If individual animals show total clinical score above 2 with 3 consecutive observation points is to be considered as CSF related clinical signs.

TABLE 7
Clinical Score Instruction

| No. | Parameters | Criteria | Score |
|---|---|---|---|
| 1 | Liveliness | Attentive (curious, stands up immediately) | 0 |
| | | Slightly reduced (stands up hesitantly, but | 1 |
| | | without help) | |
| | | Tired, gets up only when forced to, lies | 2 |
| | | down again | |
| | | Dormant, will not stand up | 3 |
| 2 | Body tension | Relaxed, straight back | 0 |
| | | Stiffness and bent back while standing up, | 1 |
| | | afterwards normal | |
| | | Bent back and stiff walking remains | 2 |
| | | Cramps | 3 |
| 3 | Body shape | Full stomach, “round” body | 0 |
| | | Empty stomach | 1 |
| | | Empty stomach, thinned body muscles | 2 |
| | | Emaciated, backbone and ribs visible, head | 3 |
| | | size too big compared to body size | |
| 4 | Breathing | Frequency 10-15/min, barely visible chest | 0 |
| | (judge before | movement | |
| | approaching | Frequency > 20/min | 1 |
| | pig) | Frequency > 20/min, distinct chest | 2 |
| | | movement | |
| | | Frequency > 30/min, breathing through open | 3 |
| | | mouth | |
| 5 | Walking | Well-coordinated movements | 0 |
| | | Hesitant walking, crossed-over legs are | 1 |
| | | corrected slowly | |
| | | Distinct ataxia/hind lameness, able to walk | 2 |
| | | Massive lameness, unable to walk | 3 |
| 6 | Skin (in | Evenly light pink, hair coat flat | 0 |
| | particular | Reddened skin areas | 1 |
| | ears, nose, | Purple-discolored and cold skin areas, few | 2 |
| | legs and | patchier | |
| | tail) | Black-red discoloration of skin, no | 3 |
| | | sensitivity, large hemorrhage in skin | |
| 7 | Eyes/ | Light pink | 0 |
| | conjunctiva | Reddened, clear secretion | 1 |
| | | Highly inflammation, turbid secretion | 2 |
| | | Highly inflammation, purulent secretion, | 3 |
| | | accentuated blood vessels | |
| 8 | Appetite | Greedy, hungry | 0 |
| | | Eats slowly when fed | 1 |
| | | Does not eat when fed, but taste food | 2 |
| | | Does not eat at all, shows no interest | 3 |
| | | for food | |
| 9 | Defecation | Soft feces, normal amount | 0 |
| | | Reduced amount of feces, dry | 1 |
| | | Only small amount of dry, fibrin-covered | 2 |
| | | feces, or diarrhea | |
| | | No feces, mucus in rectum, or watery or | 3 |
| | | bloody diarrhea | |

### Leukocytes Count

Approximately 1 mL blood samples were collected from each piglet on DPC 0, 3, 5, 7, and 16. Samples were collected using suitable needles and syringes and transferred to EDTA coated tubes. Leucocytes from blood samples were analyzed immediately by Exigo-61812 automatic blood instrument. Each sample was tested twice.

### Necropsy

Any humanely terminated/found dead animals were necropsied by investigator or designee. All surviving piglets were humanely terminated and necropsied on DPC 16. The main target tissues/organs, e.g. tonsil, kidney, spleen, lymph nodes, ileum/rectum, etc. were examined for CSF related pathogenicity. Clinical sign related CSF associated gross lesion was fixed in formalin for possible future evaluation. The fresh tonsil was collected from each piglet for CSFV virus isolation.

Spleen infarction, ulceration of intestine mucosa in rectum and ileum, haemorrhage in kidneys, lymph nodes, sub cutis petechial and mucosa of urinary bladder are of gross pathological findings of CSFV infection. A pig with 3 aforementioned findings was considered infected by CSFV. The absence of the aforementioned findings was considered not infected by CSFV. The animals with 1 or 2 aforementioned findings were considered positively infected by CSFV if its CSFV virus isolated positive

### Morbidity and Mortality

Morbidity: if individual animals meet below criteria, they are to be considered morbid. A) CSF related clinical signs within window of onset fever (one day before fever+fever day+one day after fever) and B) CSF associated gross lesions.

Mortality: Mortality associated with CSF infection.

### Study Results

### Body Temperature

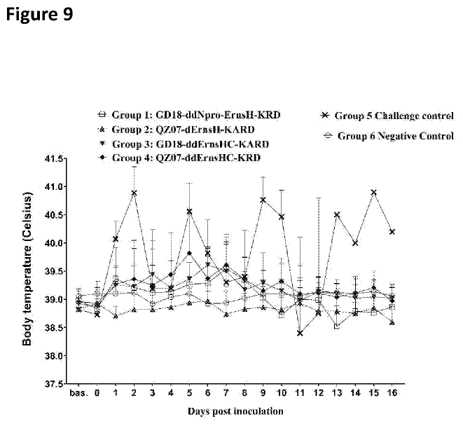

The individual normal temperature was the mean temperature of three days prior to challenge (D12 to D14). The rectal temperature results were shown in FIG. 9.

One animal in the QZ07-ddErnsHC-KRD group and two animals in the GD18-ddNpro-ErnsH-KRD group developed fever after challenge, however, all of the three piglets recovered soon. No fever was noticed in other two treatment groups.

### Clinical Observation

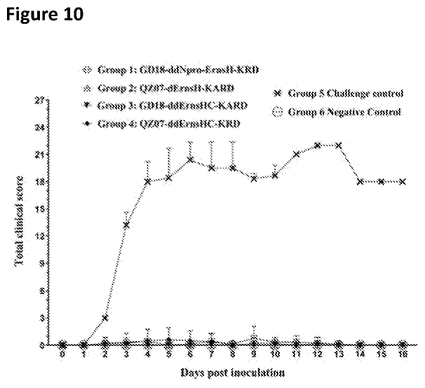

The total clinical results were shown in FIG. 10. In challenge control group, animals developed clinical sign associated with CSFV infection from DPC 2, and increased dramatically thereafter, scored up to 21 from DPC 4 to end of the study. Typical CSFV signs included, e.g., cannot stand up, cramps, unable to walk and eat, conjunctivitis, floor with very dry feces. In the QZ07-ddErnsHC-KRD group, two animals showed signs. In the GD18-ddNpro-ErnsH group-KRD, three animals showed signs. No clinical sign was observed in other two treatment groups.

### Leukocytes Count

Leucocyte counts of animals in challenge group decreased dramatically from DPC 0 to DPC 5, and stayed low till the end of the study. On the contrary, leucocyte counts of animals in all IVP groups decreased slightly from DPC 0 to DPC 5, were stable and consistently above 8*10⁹/L throughout the study.

### Gross Pathology

All piglets were necropsied when they died or at the end of the study. Group gross pathological observations were present in the Table 8. All animals in challenge control group showed at least three typical CSF lesion. Erythema in tonsil, spleen infarction, haemorrhage in kidney, haemorrhage in sub cutis, and haemorrhage in lymph nodes were commonly observed in animals in this group. No gross pathological change was observed in all treatment groups.

Morbidity, Mortality and further Efficacy Parameters

The results of efficacy assessment were summarized in Table 8.

TABLE 8
Efficacy performance summary

| | | Clinical | Gross | Mor- | Mor- |
|---|---|---|---|---|---|
| Group | Fever | signs | pathology | bidity | tality |
| QZ07-sdErnsH-KARD | 0/10 | 0/10 | 0/10 | 0/10 | 0/10 |
| GD18-ddErnsHC-KARD | 0/10 | 0/10 | 0/10 | 0/10 | 0/10 |
| QZ07-ddErnsHC-KRD | 1/10 | 2/10 | 0/10 | 0/10 | 0/10 |
| GD18-ddNrpo-ErnsH-KRD | 2/9 | 3/9 | 0/9 | 1/9 | 0/9 |
| Challenge control | 10/10 | 10/10 | 10/10 | 10/10 | 10/10 |
| Negative control | 0/5 | 0/5 | 0/5 | 0/5 | 0/5 |

Compared with the challenge control group, all the recombinant viruses provide significant protection. All animals of the “Negative control”, which did not receive any of the test vaccines nor the challenge wild-type CSFV material, remained healthy.

### Conclusion

Efficacy study showed protection from each candidate, no mortality was seen in each group. Only a small portion of piglets showed transient fever and clinical sign after challenge, however they recovered soon.

### Example 5: Development of DIVA ELISA Based on mAb 6B8 Specific Epitope

The objective of this Example is to develop a CSFV DIVA ELISA method which can differentiate the infected animals from animals vaccinated with mAb 6B8 epitope based candidate DIVA vaccine.

### Materials and Reagents for DIVA ELISA

96-well ELISA plate: Corning; Cat. No. 42592.

96-well cell culture plate: Corning; Cat. No. 3595.

Coating buffer (CBS): 0.05M Na₂CO₃, 0.05M NaHCO₃; pH9.6.

Substrate solution: TMB, Invitrogen; Cat. No. 002023.

Stop solution: 1N HCl.

Washing buffer: PBST with 0.1% Tween-20.

Blocking buffer: PBST with 5% skimmed milk.

Dilution buffer: PBST with 5% skimmed milk.

Coating antigen: CSFV E2 protein (E2 from QZ07, full length), which was named as QZ07-spE2-delTM (4.22 mg/ml), produced and purified by the inventors.

Competitive protein: CSFV E2 protein (Full length) with the KARD mutation in 6B8 epitope (named as QZ07-spE2-delTM-6B8-KARD, 0.549 mg/ml), produced and purified by the inventors.

Swine serum samples to be tested: serum from safety and efficacy studies of QZ07-sdErnsH-KARD.

Blocking antibody: Monoclonal antibody 6B8.

Secondary antibody: HRP conjugated, goat anti-mouse IgG: Santa Cruze; Cat No. SC-2005.

Competitive ELISA (cELISA)

The coating antigen was diluted with coating buffer to a final concentration of 2.0 μg/ml, and added to a 96-well ELISA plate with 100 μl per well of the diluted coating antigen. The plate was sealed and incubated at 4° C. overnight. The plate was washed with 300 μl/well washing buffer for 4 times, and then blocked with 200 μl/well blocking buffer at 37° C. for 1 hours. After blocking, the plate was washed with 300 μl/well washing buffer for 4 times. 50 μl of serum samples mixed with 50 μl dilution buffer was added into the ELISA plate at 37° C. for 2 hours. The plate was washed with 300 μl/well washing buffer for 4 times. The blocking antibody mAb 6B8 was diluted at 1:800 with dilution buffer, and 100 μl/well was added to the plate, incubated at 37° C. for 1 hour. The ELISA plate was washed with 300 μl/well washing buffer for 4 times. Then, the secondary antibody diluted at 1:10000 in dilution buffer was added, 100 μl/well, incubated at 37° C. for 1 hour. The ELISA plate was washed with 300 μl/well washing buffer for 4 times. 100 μl of substrate solution (TMB) was added into each well, incubated for 10 minutes at room temperature without placing the plate in direct light. 100 μl of stop solution (1N HCl) was added into each well. OD₄₅₀ was read and the blocking rate was calculated according to the following formula:

$$BLOCKING RATE ( \% ) = \frac{average negative control ( OD 450 ) - average sample ( OD 450 )}{averager negative control ( OD 450 )} * 100$$

Double Competitive ELISA (dcELISA)

The coating antigen was diluted with coating buffer to a final concentration of 2.0 μg/ml, and added to a 96-well ELISA plate with 100 μl per well of the diluted coating antigen. The plate was sealed and incubated at 4° C. overnight.

The ELISA plate was washed with 300 μl/well washing buffer for 4 times, and then blocked with 200 μl/well blocking buffer at 37° C. for 1 hours. After blocking, the plate was washed with 300 μl/well washing buffer for 4 times.

The serum sample was pre-treated with the competitive protein. In brief, the competitive protein was diluted with dilution buffer to 1 μg/60 μl, meanwhile, 60 μl dilution buffer was used as control. 60 μl of the diluted competitive protein was combined with 60 μl of serum samples, incubated in a 96-well cell culture plate at 37° C. for 2 hours.

100 μl of the pre-treated serum samples was transferred into the blocked ELISA plate at 37° C. for 2 hours. The plate was washed with 300 μl/well washing buffer for 4 times. The blocking antibody mAb 6B8 was diluted at 1:800 with dilution buffer, and 100 μl/well was added to the ELISA plate, incubated at 37° C. for 1 hour. The ELISA plate was washed with 300 μl/well washing buffer for 4 times. Then, the secondary antibody diluted at 1:10000 in dilution buffer was added, 100 μl/well, incubated at 37° C. for 1 hour. The ELISA plate was washed with 300 μl/well washing buffer for 4 times. 100 μl of substrate solution (TMB) was added into each well, incubated for 10 minutes at room temperature without placing the plate in direct light. 100 μl of stop solution (1N HCl) was added into each well. OD₄₅₀ was read and the blocking rate was calculated according to the following formula:

$$BLOCKING RATE ( \% ) = \frac{average negative control ( OD 450 ) - average sample ( OD 450 )}{averager negative control ( OD 450 )} * 100$$

Different combinations of DIVA mutations in 6B8 epitope are feasible for DIVA Sera from overdose safety studies of candidates with RD (positions 24 and 25), KRD (positions 14, 24 and 25) or KARD (positions 14, 22, 24 and 25) mutation in 6B8 epitope on different attenuation backgrounds (QZ07-sdErnsH or GD18-ddNpro-ErnsH) were tested using the above described dcELISA, pre-treated with various amount of competition protein.

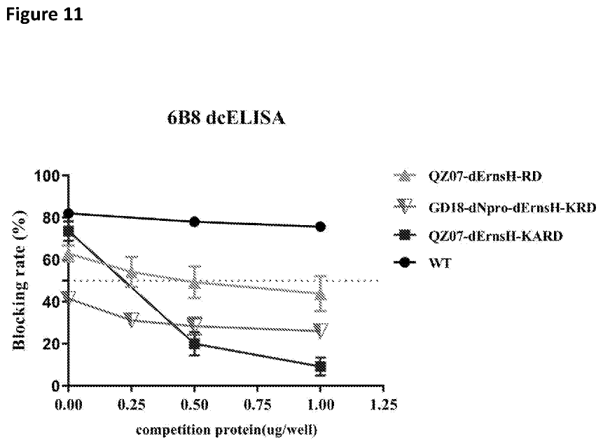

Results were shown in FIG. 11. Overdose convalescent sera derived from either RD, KRD or KARD candidate vaccination showed low blocking rate (lower than 40%) while WT derived serum remain high blocking rate (above 50%). Thus each combinations of 6B8 epitope mutations showed feasibility to generate a DIVA candidates.

Comparison of cELISA and dcELISA for DIVA

Sera samples from previous safety evaluation study (0, 7, 14 and 21 days post vaccination) and previous efficacy evaluation study (0, DPC0 and DPC16) of candidate QZ07-sdErnsH-KARD were used to compare the effects of cELISA and dcELISA for DIVA.

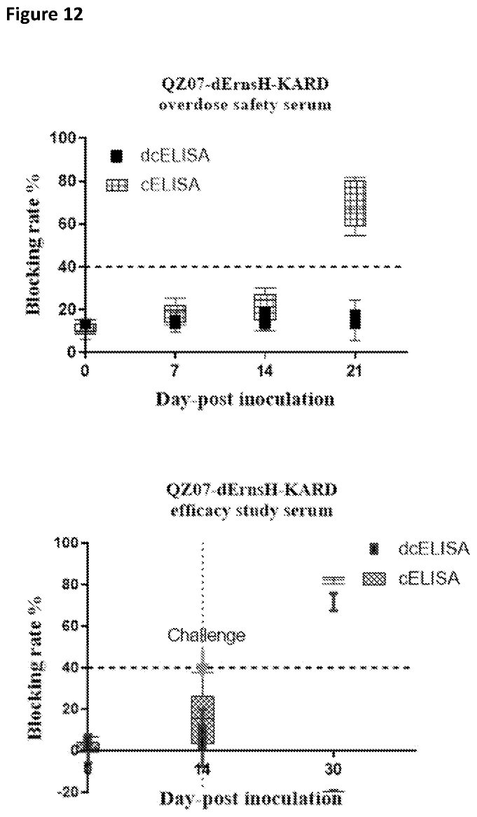

Results were shown in FIG. 12. The dcELISA results showed low blocking rate against 6B8 in the sera samples of animals vaccinated with the QZ07-sdErnsH-KARD candidate in safety evaluation study. In the efficacy evaluation study, high blocking rates were observed after Shimen field strain challenge, whereas, low blocking rates were observed in the sera before challenge (DPC0). Therefore, animals infected with CSFV can be well differentiated from animals vaccinated with the QZ07-sdErnsH-KARD DIVA vaccine candidate by dcELISA.

However, by using cELISA, high blocking rates were also observed in the sera samples from safety evaluation study which were not challenged (21 days post vaccination). Thus, cELISA is not suitable for DIVA of this candidate.

### Example 6: Immunoinfluoscent Assay (IFA) for Determining the Binding of 6B8 mAb to a Mutated 6B8 Epitope

The binding of 6B8 mAb to a mutated 6B8 epitope (Test Sample) is determined by an immunoinfluoscent assay (IFA) according to the following steps:

1. In a 96-well microtiter plate PK-15 cells at about 1.1×10⁴ cells/well are seeded and infected with the following CSFV at MOI of 0.001 to 0.01, each in duplicates:
(i) Test Sample: CSFV containing E2 protein with a modified 6B8 epitope;
(ii) Positive Control: CSFV containing an E2 protein with the wildtype 6B8 epitope (such as the attenuated field stain QZ07-dErnsH or GD18-ddNrpo-ErnsH);
(iii) Negative Control: CSFV containing an E2 protein with the KARD mutation within the 6B8 epitope as described herein (such as for example QZ07-sdErnsH-KARD).

The CSFV inoculated cells are held in the incubator under 37° C., CO₂ (4 to 6%), for 3 days with no fluid change or addition of medium.

2. The culture media is discarded, and the cells are rinsed twice with 1×PBS (200 to 250 μL/well).

3. For fixation of the CSFV infected cells, 4% formaldehyde is added into the assay plates (50 μL/well).

4. The plates are incubated at room temperature for 30-40 minutes. In fume hood, the formaldehyde is discarded gently, and the cells are washed once with 1×PBS (200 to 250 μL/well).

5. For permeabilization of the cell membrane of the CSFV infected cells, 0.1% Triton X-100 is added into each well, 50 μL/well. The assay plates are incubated at room temperature for 15-20 minutes. The assay plates are washed twice with 1×PBS (200 to 250 μL/well).

6. The 6B8 specific mAb (such as the antibody produced by a hybridoma deposited at CCTCC under the accession number CCTCC C2018120) is diluted with PBS containing 5% BSA to 1:500 to 1:1000, then added to the assay plates with 50 μL/well. The plates are covered with the lid and incubated at 37° C. for 1-2 hour.

7. The assay plates are rinsed 3 times with 1×PBS (250 μL/well).

8. The secondary antibody, Alexa Fluor® 488 conjugated Donkey anti-mouse antibody that specifically binds to the 6B8 antibody (ThermoFisher, Invitrogen, cat #21202), is diluted with PBS containing 5% BSA at 400 fold, added to the assay plates with 50 μL/well. The plates are covered with the lid and incubated at 37° C. for 1-2 hour.

9. The assay plates are rinsed 3 times with 1×PBS (250 μL/well). At last, 1×PBS is added, 100 μL/well. Final fluorescence signals are read out with an inverted fluorescence microscopy.

A negative result of the Test Sample in this IFA (in both replicates) indicates that the one or more mutations within the 6B8 epitope of the E2 protein leads to a specific inhibition of the binding of a 6B8 monoclonal antibody to such mutated 6B8 epitope.

### Example 7: Safety Study in Pregnant Sows

Sows of the same age and origin were vaccinated according to following Table 10 at Day 55 of gestation and necropsied at Day 100 of gestation.

TABLE 10

| | No. of | Vaccination | | |
|---|---|---|---|---|
| Group | animals | Treatment | Route | Dose |
| 1 | 8 | GD18-ddErnsHC-KARD | IM | 1 mL, 1 × 10⁵ |
| | | | | TCID50 |
| 2 | 8 | QZ07-ddErnsHC- KARD | IM | 1 mL, 1 × 10⁵ |
| | | | | TCID50 |
| 3 | 8 | GD18-ddNpro-ErnsH- KARD | IM | 1 mL, 1 × 10⁵ |
| | | | | TCID50 |
| 4 | 8 | QZ07-sdErnsH- KARD | IM | 1 mL, 1 × 10⁵ |
| | | | | TCID50 |
| 5 | 3 | Alfort-Erns H297K | IM | 1 mL, 1 × 10⁵ |
| | | (positive control) | | TCID50 |
| 6 | 5 | n/a | n/a | n/a |
| | | (negative control) | | |

All sows were negative for antibodies against pestiviruses before start of the study. All sows of the negative control group remained antibody- and antigen-negative for CSFV until end of the study.

No vaccination associated abnormality was observed in the study: i) no injection site reactions were noticed in any of the animals; ii) none of the animals showed a temperature above 38.8C at any time point; iii) no vaccination associated clinical sign in four vaccination groups; iv) no viremia was detected in sows of four vaccination groups while viremia was detected between 7-14 dpi in the positive control group.

As shown in Table 11, vaccination had no impact on reproductive performance.

TABLE 11

| | | Sow performance | | |
|---|---|---|---|---|
| | | | | Normal |
| | | | No of | fetuses/ |
| | No. of | | normal | Total |
| Group | animals | Treatment | fetuses | fetuses (%) |
| 1 | 8 | GD18-ddErnsHC-KARD | 142 | 142/143 |
| | | | | (99.30%) |
| 2 | 8 | QZ07-ddErnsHC-KARD | 133 | 133/134 |
| | | | | (99.25%) |
| 3 | 8 | GD18-ddNpro-ErnsH-KARD | 124 | 124/124 |
| | | | | (100%) |
| 4 | 8 | QZ07-sdErnsH-KARD | 124 | 124/128 |
| | | | | (96.87%) |
| 5 | 3 | Alfort-Erns H297K | 8 | 8/44 |
| | | (positive control) | | (18.18%) |
| 6 | 5 | n/a | | 65/80 |
| | | (negative control) | | (81.25%) |

Note:
One sow in Group 6 had dystocia and resulted in death of 9 piglets.

Based on the above preliminary results, vaccination with the four candidates are safe in pregnant sows.

1. A recombinant CSFV (classical swine fever virus) comprising at least one mutation within the 6B8 epitope of the E2 protein, wherein the unmodified 6B8 epitope is specifically recognized by the 6B8 monoclonal antibody.

2. The recombinant CSFV according to claim 1, wherein
i) the at least one mutation within the 6B8 epitope of the E2 protein leads to a specific inhibition of the binding of a 6B8 monoclonal antibody to such mutated 6B8 epitope;
ii) the 6B8 monoclonal antibody
(a) is produced by a hybridoma deposited at CCTCC under the accession number CCTCC C2018120,
(b) comprises a heavy chain variable region (VH) having an amino acid sequence as set forth in SEQ ID NO: 9 and a light chain variable region (VL) having an amino acid sequence as set forth in SEQ ID NO: 10,
(c) comprises the CDRs of the monoclonal antibody produced by a hybridoma deposited at CCTCC under the accession number CCTCC C2018120, or
(d) comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30;
iii) the 6B8 epitope of the E2 protein specifically recognized by the 6B8 monoclonal antibody is defined at least by
(a) the amino acid residue at position 14, position 22, position 24 and/or positions 24/25 of the E2 protein;
(b) the amino acid residue S14, G22, E24, and/or E24/G25 of the E2 protein, or the amino acid residue S14, G22, G24, and/or G24/G25 of the E2 protein; or
(c) the amino acid sequence STNEIGPLGAEG or STDEIGLLGAGG;
iv) the recombinant CSFV comprises
(a) a substitution at amino acid position 24 of the E2 protein, a substitution at amino acid positions 24/25 of the E2 protein, a substitution at amino acid position 14 of the E2 protein, and/or a substitution at amino acid position 22 of the E2 protein;
(b) a substitution at amino acid position 24 of the E2 protein to R or K, substitutions at amino acid positions 24/25 of the E2 protein to R/D or K/D, a substitution at amino acid position 14 of the E2 protein to K, Q or R, and/or a substitution at amino acid position 22 of the E2 protein to A, R, Q or E, with A and R being preferred;
(c) a substitution at amino acid position 24 of the E2 protein from E or G to R or K, substitutions at amino acid position 24 of the E2 protein from E or G to R or K and at amino acid position 25 of the E2 protein from G to D, a substitution at amino acid position 14 of the E2 protein from S to K, Q or R, and/or a substitution at amino acid position 22 of the E2 protein from G to A, R, Q or E, with A and R being preferred; or
(d) at least one mutation in the Erns protein and/or at least one mutation in the Npro protein; preferably such mutation in the Erns protein is a deletion of amino acid at amino acid position 79 of Erns protein and/or a deletion of amino acid at amino acid position 171 of Erns protein, and the mutation in Npro protein is a deletion of the Npro protein except for the first four amino terminal amino acids;
v) the at least one mutation within the 6B8 epitope of the E2 protein results in a mutated 6B8 epitope sequence of any one of SEQ ID Nos: 13-14 and 31-34; or
vi) the recombinant CSFV is attenuated.

3.-12. (canceled)

13. The recombinant CSFV according to claim 1, wherein the recombinant CSFV is derived from
i) C-strain or a field strain QZ07, GD191, or GD18;
ii) a field strain QZ07, and
(a) comprises a deletion of amino acid at amino acid position 79 of Ems protein, and
(b) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein;
iii) a field strain QZ07, and
(a) comprises a deletion of amino acid at amino acid position 79 of Ems protein, a deletion of amino acid at amino acid position 171 of Ems protein, and
(b) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein;
iv) a field strain GD18, and
(a) comprises a deletion of amino acid at amino acid position 79 of Ems protein, a deletion of the Npro protein except for the first four amino terminal amino acids, and
(b) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein; or
v) a field strain GD18, and
(a) comprises a deletion of amino acid at amino acid position 79 of Ems protein, a deletion of amino acid at amino acid position 171 of Ems protein, and
(b) a substitution of E to R or K at amino acid position 24 of the E2 protein, or a substitution of E to R or K at amino acid position 24 and a substitution of G to D at amino acid position 25 of the E2 protein, and optionally further comprises a substitution of S to K, Q or R at amino acid position 14 of the E2 protein and/or a substitution of G to A, R, Q or E, with A and R being preferred, at amino acid position 22 of the E2 protein.

14.-17. (canceled)

18. An isolate nucleic acid coding for a recombinant CSFV according to claim 1.

19. A vector comprising the nucleic acid of claim 18.

20. An immunogenic composition comprising the recombinant CSFV according to claim 1, an isolate nucleic acid coding for the recombinant CSFV according to claim 1, or a vector comprising the isolate nucleic acid.

21. The immunogenic composition according to claim 20, wherein said immunogenic composition is a marker vaccine or a DIVA (differentiation between infected and vaccinated animals) vaccine.

22. (canceled)

23. A method of preventing and/or treating diseases associated with CSFV in an animal, the method comprising the step of administering the immunogenic composition according to claim 20 to an animal in need thereof.

24. A method of marking a CSFV vaccine comprising the recombinant CSFV of claim 1, comprising introducing into a CSFV at least one mutation within the 6B8 epitope of the E2 protein specifically recognized by the 6B8 monoclonal antibody.

25.-33. (canceled)

34. A method of differentiating animals infected with CSFV from animals vaccinated with the immunogenic composition of claim 20, comprising
a) obtaining a sample, and
b) testing said sample in an immuno test, wherein the immuno test
i) comprises testing whether an antibody specifically recognizing the 6B8 epitope of the CSFV E2 protein can bind to the CSFV E2 protein in the sample;
ii) comprises testing whether an antibody specifically recognizing a 6B8 epitope of the CSFV E2 protein is present in the sample, and/or testing whether an antibody specifically recognizing a mutated 6B8 epitope of the CSFV E2 protein is present in the sample; or
iii) is an EIA (enzyme immunoassay) or ELISA (enzyme linked immunosorbent assay), preferably a double competitive ELISA.

35. (canceled)

36. The method according to claim 34, wherein the antibody specifically recognizing the 6B8 epitope
i) is produced by a hybridoma deposited at CCTCC under the accession number CCTCC C2018120, or
ii) comprises a heavy chain variable region (VH) having an amino acid sequence as set forth in SEQ ID NO: 9 and a light chain variable region (VL) having an amino acid sequence as set forth in SEQ ID NO: 10, or
iii) comprises the CDRs of the monoclonal antibody produced by a hybridoma deposited at CCTCC under the accession number CCTCC C2018120, or
iv) comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

37.-38. (canceled)

39. An antibody or an antigen-binding fragment thereof, wherein said antibody is produced by a hybridoma deposited at CCTCC under the accession number CCTCC C2018120, or wherein said antibody comprises a heavy chain variable region (VH) having an amino acid sequence as set forth in SEQ ID NO: 9 and a light chain variable region (VL) having an amino acid sequence as set forth in SEQ ID NO: 10, or wherein the antibody comprises the CDRs of the monoclonal antibody produced by a hybridoma deposited at CCTCC under the accession number CCTCC C2018120, or wherein the antibody comprises a VH CDR1 comprising the amino acid sequence set forth in SEQ ID NO:25, a VH CDR2 comprising the amino acid sequence set forth in SEQ ID NO:26, a VH CDR3 comprising the amino acid sequence set forth in SEQ ID NO:27, a VL CDR1 comprising the amino acid sequence set forth in SEQ ID NO:28, a VL CDR2 comprising the amino acid sequence set forth in SEQ ID NO:29, and a VL CDR3 comprising the amino acid sequence set forth in SEQ ID NO:30.

40. A kit for differentiating animals infected with CSFV from animals vaccinated with an immunogenic composition comprising a recombinant CSFV (classical swine fever virus) comprising at least one mutation within the 6B8 epitope of the E2 protein, wherein the unmodified 6B8 epitope is specifically recognized by the 6B8 monoclonal antibody, wherein the kit comprises the antibody of claim 39, or an antigen-binding fragment thereof.

41. An recombinant attenuated CSFV, wherein the recombinant attenuated CSFV has at least one mutation in the Ems protein and/or at least one mutation in the Npro protein; preferably such mutation in the Ems protein is a deletion of amino acid at amino acid position 79 of Ems protein and/or a deletion of amino acid at amino acid position 171 of Ems protein, and the mutation in Npro protein is a deletion of the Npro protein except for the first four amino terminal amino acids.

42. The recombinant attenuated CSFV according to claim 41, wherein the recombinant attenuated CSFV
i) is derived from C-strain or a field strain QZ07 or GD18;
ii) is derived from a field strain QZ07, and comprises a deletion of amino acid at amino acid position 79 of Ems protein;
iii) is derived from a field strain QZ07, and comprises a deletion of amino acid at amino acid position 79 of Ems protein, and a deletion of amino acid at amino acid position 171 of Ems protein;
iv) is derived from a field strain GD18, and comprises a deletion of amino acid at amino acid position 79 of Ems protein, and a deletion of the Npro protein except for the first four amino terminal amino acids; or
v) is derived from a field strain GD18, and comprises a deletion of amino acid at amino acid position 79 of Ems protein, and a deletion of amino acid at amino acid position 171 of Ems protein.

43.-46. (canceled)

47. An isolate nucleic acid coding for a recombinant attenuated CSFV according to claim 41.

48. A vector comprising the nucleic acid of claim 47.

49. An immunogenic composition comprising the recombinant attenuated CSFV according to claim 41, an isolate nucleic acid coding for the recombinant attenuated CSFV according to claim 41, or a vector comprising the isolate nucleic acid.

50. (canceled)

51. A method of preventing and/or treating diseases associated with CSFV in an animal, the method comprising the step of administering the immunogenic composition according to claim 49 to an animal in need thereof.

52. A method of making a recombinant attenuated CSFV vaccine comprising a recombinant attenuated CSFV of claim 41, comprising introducing into a CSFV at least one mutation in the Erns protein and/or at least one mutation in the Npro protein; preferably such mutation in the Ems protein is a deletion of amino acid at amino acid position 79 of Ems protein and/or a deletion of amino acid at amino acid position 171 of Ems protein, and the mutation in Npro protein is a deletion of the Npro protein except for the first four amino terminal amino acids.

53.-57. (canceled)


SEQUENCE LISTING (35 sequences)

SEQ ID NO: 1 - DNA, 12296 bp, Artificial Sequence
  QZ07-wt
gtatacgaggttagttcgttctcgtgtacaatattggacaaaacaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggcgctgcagaggcccactaacaggctagtataaaaatctctgctgtacatggcacatggagttgaatcattttgaactattgtataaaacaaacaaacaaaaaccaatgggagtggaggaaccggtgtatgacatcgcagggagaccattatttggggacccaagtgaggtacacccacaatcaacactgaagctaccacacgacaggggaagaggtaatatcagaactacactgaaggacctacctaggaaaggcgactgcaggagtggaaaccacctaggaccagttagtgggatatacgtaaagcctggccctgtcttctatcaggactacatgggccctgtctaccatagagccccattagagttttttggtgaggcccaattttgtgaggtgaccaagaggataggtagggtgacgggtagtgacggaaagctctaccacatatatgtgtgcatcgacggttgcatactgttgaagttagccaaaaggggcgcgcccagatccctaaagtggactaggaacttcaccgactgtccactgtgggtcactagttgttctgatgatggcacaggtgacagcaaggataaaaagccagacaggatgaacaagggtaaattaaagatagccccaaaagagcatgagaaggacagtaagaccaagccacctgatgctacaattgtggtagagggagtaaaataccaagtaaaaaagaaaggcaaagtcaaagggaagaacactcaagatggcctgtaccataataagaacaaaccaccagagtccaggaagaaactagaaaaagccctattggcctgggcagtgatagcaatagtgctgtaccagcctgtagcagccgagaatataacccaatggaacctgagtgacaacggcacaagcggcatccagcaagctatgtatctcagaggggtcaataggagcttgcatgggatctggcctgaaaaaatatgcaagggggtccctactcatctggccactgacacggaactgacagagatacgcgggatgatggacgccagtgagaggacgaactacacgtgttgtaggttgcagagacacgaatggaacaaacatggctggtgcaactggtacaacatagacccctggattcagttaatgaacaggacccaagcaaatttgacagaaggccctccagataaagagtgtgccgtgacttgcaggtatgacaaaaataccgatgttaacgtggtcacccaggccaggaatagacctactactctgactggctgcaagaaagggaaaaatttttcatttgcaggtacggtcatagagggcccatgcaatttcaacgtatccgtggaggacatcttatatggcgaccatgagtgtggcagcctgttccaggacacggctctgtacctattagatggaatgaccaacactatagagaaagccaggcagggtgcggcaagagtcacatcttggctcgggagacaactcagaaccacagggaagaagttggagagaggaagcaaaacctggttcggtgcctatgccctgtcaccttactgcaatgtaacaaggaaaatagggtacatatggtacacgaacaattgcactccggcatgcctcccaaaaaacacaaaaataataggcccaggaaagtttgacaccaatgcagaagacgggaagattcttcatgaaatggggggccacctatcagaattcttgttgctttctctggtagtcctgtctgacttcgccccagaaacggccagtacattatacctaatcttacactatgcaatccctcagtcccgtgacgaacccgaggtttgcgatacaaaccagctcaacctaacagtgggacttaggacagaagatgtggtaccatcatcagtctggaatattggcaaatatgtgtgtgttagaccagattggtggccgtatgaaactaaggtggctctactgtttgaagaggcgggacaggtcataaagttagctctacgggcactgagggatttaactagagtctggaacagtgcatcaactactgcgttcctcatctgcttgataaaaatattaagaggacaggttgtgcaaggtataatatggctgctgctggtgactggggcacaagggcggttgtcctgcaaggaagactacaggtatgcgatatcatcaaccaatgagatagggccgctaggggctgaaggtctcaccaccacctggaaagaatataaccatggtttgcagctggatgacgggactgtcagggccatttgcactgcagggtcctttaaagttatagcacttaatgtggtcagtaggaggtacctggcatcattacacaagagggctttacccacttcagtaacatttgaactcctatttgatgggactagtccagcaattgaggagatgggagatgactttggatttgggctgtgcccttttgacacaacccccgtggtcaaagggaagtacaataccactttattaaacggtagtgctttctatctagtctgcccaataggatggacgggtgtcatagagtgcacggcagtaagccctacaaccttgagaacagaagtggtgaagaccttcaagagagagaagcctttcccacacagagtgggttgcgtgaccactgttgtagaaaaagaagacctgttctactgcaagtgggggggtaattggacatgtgtaaaaggcaacccggtgacctacatgggggggcaagtaaaacaatgcagatggtgcggttttgacttcaaggagcccgatgggctcccacactaccccataggcaagtgcatcctagcaaatgagacgggttacagggtagtggattccacagactgcaatagagatggcgtcgttatcagcactgaaggtgaacacgagtgcttgattggcaacaccaccgtcaaggtgcacgcgttggatggaagactgggccctatgccgtgcagacccaaagaaatcgtctctagcgcaggacctgtaaggaaaacttcctgcaccttcaactacacaaagacactaagaaacaagtactatgaacccagggacagctatttccagcaatatatgcttaagggcgagtaccaatactggtttgatctggacgtgaccgaccaccacacagactactttgccgaatttgttgtcttggtggtagtggcactattaggggggaggtacgtcctatggctaatagtgacctatgtaattttaacagagcaactcgctgccggtttacagctgggccaaggcgaggtagtactgatagggaacttaattacccacacggacaatgaggtagtggtatacttcttactgctctacctaataataagagacgagcctataaagaaatggatactactgctgtttcatgccatgaccaacaacccagttaagaccatgacagtagcattgctgatgatcagtggggtcgccaagggtgggaaaacagatggtggttggcagaggcagccggagaccaattttgacattcaactcgcactggcagttatagtagtcgttgtgatgttactggcaaagagagacccgactacttttcccctggtgatcacagtggcaaccttaagaacagctaagataaccaatggtttcagcacagatctagctatagccacagtgtcggcagctttgctaacctggacttatatcagtgactactacaaatataagacttggctacagtacctcatcagcacggtgactgggatcttcctaataagggtactgaagggaataggcgagctagacatgcacgccccaactttgccatctcacagaccccttttctacatcctcgtgtacctcatctccactgctgtggtgactagatggaacctggacgtagccggactgctgctacagtgtgtcccaacacttctaatggtgttcacgatgtgggctgacattctcaccctaatcctcgtgttacccacttatgaactggcaaagttgtattaccttaaggaagtgaagatcggaaccgaaagaggatggctgtggaaaactaactataagagggtaaatgacatctatgaagtcgaccaagctggcgagggggtctacctcttcccttcgaaacaaaagactagtgctataacaagcaccatgttgccattgatcaaagccatactcattagctgcatcagtaacaagtggcaactcatatatttactgtacctgatattcgaagtgtcctactaccttcacaagaaagtcatagatgaaatagctggtgggaccaacttcgtttcaaggctagtggcagccttaattgaagttaattgggccttcgacaatgaagaagttaaaggcctaaagaagttctttttgctgtccagtagagttaaagaattggtcatcaaacacaaagtgaggaatgaagtagtggcccgctggttcggagatgaagagatctatgggatgcccaagttgatcggcttagtcaaggcagcaactctaagtaaaaataaacactgtatcttgtgcaccgtctgcgaggacagagattggagaggagaaacctgcccaaaatgtgggcgtttcggaccaccagtgatctgtggtatgaccctagccgacttcgaggagaagcattataagaggatattcatcagggaggaccaatcagatggaccgctcagggaggagcgtgcagggtacttacagtacagagctaggggtcaactgtttctgaggaatctcccagtgttggcgacaaaagtcaagatgctcctggttggtaacctcgggacggaggttggggatttggagcaccttggctgggtgcttagagggcctgctgtctgcaagaaggttactgaacatgaaaagtgtgctacgtctataatggataagttgactgctttcttcggcgttatgccaagaggtaccacccccagagcccccgtgagatttcccacctccctcctaaagataagaagagggctagagacgggttgggcttacacacatcaaggtggcattagctcagttgaccatgtgacttgtgggaaagacttgctggtgtgtgacactatgggccggacaagggtcgtttgccaatcaaataataagatgactgacgaatccgaatacggggtcaaaaccgactcagggtgcccagaaggggccagatgttatgtgttcaaccctgaggcagttaacatatcaggcactaaaggggccatggtccacttacaaaaaactggtggggagttcacctgcgtaacagcatcaggaaccccggccttctttgatctcaagaacctcaagggctggtcagggctaccgatattcgaagcatcgagtggaagggtagtcggaagggtcaaggtcgggaagaacgatgactctaaaccaaccaaactcatgagtgggatacaaacggtctctaaaagtgccactgacctgacggagatggtaaagaagataacaactatgaacaggggagagttcagacagataaccttggccacaggtgctgggaaaactacagagctccccaggtcagtcatagaagagatagggagacacaaaagggtgttggtattgatccccttgagggccgcagcagaatcagtataccaatatatgaggcagaaacacccaagtatagcattcaacctgaggataggggagatgaaggaaggtcacatggccacggggataacctatgcctcctacggttacttttgccagatgccacaacctaagctgagggccgcaatggtagagtactcctacatctttctagatgagtaccattgtgccaccccagaacaactggctatcatggggaagatccacagattctcagagaacctgcgggtggtagctatgacagcgacaccagcagggacagtaacaactactgggcagaaacaccctatagaggagttcatagccccggaagtaatgaaaggagaagacttaggttcagagtacttagacattgccggactaaagataccagtagaggagatgaagaacaacatgctagtttttgtaccaactaggaatatggcggtagaggcggcaaagaaactgaaagctaaaggatacaactcaggctattactacagtggtgaggatccatctaacctgagggtagtcacatcgcagtccccatacgtggttgtagcgaccaacgcgatagaatcaggcgtcactctcccggaccttgacgtggtcgttgacacgggactcaagtgtgagaaaagaatccgattgtcacccaagatgcctttcatagtgactggcctaaaaagaatggctgttaccattggggaacaagcccagagaagagggagggttggaagagtaaagcccgggagatactacaggagccaagaaacccctgtcggctctaaggactaccactacgatttattgcaagcccagaggtacggtatagaagatgggataaatatcaccaaatccttcagagaaatgaactatgattggagcctctatgaggaagatagcctgatgataacacaattggaaatcctcaacaatctactgatatcagaggagctgccggtggcagtaaaaaatataatggctaggactgaccacccagaaccaattcaactagcatataatagctacgagacacaagtgcctgtgttgttcccaaaaataagaaatggagaggtgactgacacttacgatacttacaccttcatcaatgcaagaaaattgggagatgatgtacctccctacgtgtatgccacagaagatgaggacttggcagtggaactgttgggcctagattggccggaccctgggaaccaaggcactgtggaggccggcagagcactaaaacaggtggtcggtctatcaacagccgagaatgccctgttagtagccttgtttggctacgtagggtaccaggccctctcaaagaggcatatacctgtggtcacagacatatactcagttgaagatcacaggctagaggacaccacacacctacagtacgctccgaatgccatcaagacggaggggaaggagactgaattgaaggagttggctcagggggatgtgcagagatgcgtggaagcgatgaccaattacgcgagagggggcatccaatttatgaagtcacaggcactgcaggtgagagaaacccccacttataaagagacaatgaatactgtagcagactatgtgaaaaagtttatcgaggcactgtctgatagcaaggaagacatcttgaaatatgggctgtggggcgtacatacggccttgtacaagagcattggtgccaggcttggtcacgaaaccgcgttcgcaaccctagccgtgaaatggctggcatttgggggggaatcaatagcagatcatataaaacaagcggctacagacttggtcgtctactatatcatcaacagacctcagttcccaggagacacagagacacaacaggaggggagaaattttgtggccagcctactggtctcagccctagcaacttacacatataaaagctggaactataataacctgtccaagatagtagaaccggctttggccaccctgccctatgccgctaaagcccttaaattatttgcccctaccaggctagagagcgtcgtcatattgagcaccgcaatctacaaaacatacctatcaatcaggcgaggcaaaagcgatggattgctaggtacaggggttagtgcggctatggagattatgtcacaaaatccggtatcagtgggcatagcagttatgctgggggttggggctgtagcagcacacaacgcaattgaagccagtgagcagaagagaacactacttatgaaagtttttgtaaagaacttcttggaccaagcagccacagatgaactagtcaaagagagccctgagaaaataataatggccttgtttgaagcagtgcaaacagtaggcaatcctcttaggctagtgtaccacctttacggggttttttataaagggtgggaagcaaaagagttggcccaaaggacagccggcaggaatcttttcaccttgataatgtttgaggccgtggaactattgggagtagatagcgaaggaaaaatccgccaactatcgagtaattacatattagagctcctgtataagttccgtgacagtatcaaatctagtgtgagggagatagcaatcagctgggcccctgccccctttagttgcgattggacaccaacagatgacagaatagggcttccccataacaattacctccaaatggagacaagatgcccttgtggctacaggatgaaagcagtaaaaacctgtgctggggagttgagacttctggaagagggaggttcattcctctgcaggaataaattcggtagagggtcacggaactatagggtgacaaaatactatgatgacaatttatcagaaataaaaccagtgataagaatggaaggacatgtggaactatattataagggggccactatcaaactggactttaacaacagtaaaacagtactggcaaccgacaaatgggaggttgaccattccaccctggtcagggcgctcaagaggcacacaggggctgggtaccaaggagcgtatatgggtgagaaacctaaccacaaacatctgatagagagagactgtgcaacgatcacaaaagacaaggtttacttcatcaagatgaagagggggtgtgcgttcacttatgacttgtccctccacaacctcacccggttaatcgaattggtacacaaaaacgacctggaagatagagaaatccctgctgttacggttacaacctggctggcctacacatttgtgaatgaagacatagggaccataaaaccagtttttggggaaaaagtaacaccggaaaaacaggaggaggtagccttgcagcctgctgtggtggtggacacaacagatgtggccgtgaccgtggtgggggaaacctctactatgactacaggggagaccccaacggcattcaccagcttaggttcagactctaaagttcaacaagttctgaagttaggggtggatgagggtcaataccccgggcccagtcagcagagagcaagcttgctcgacgctatacaaggcgtggatgaaaggccctcggtattgatattgggatctgataaggccacctccaatagggtgaagaccgcaaagaatgtcaagatattcaggagcagagaccccctggaactgagagaaatgatgagaagagggaagatcctggtcatagccctatgtaaggtcgacactgctctactgaaatttgttgattacaaaggcaccttcctgaccagagaaaccctagaggcattaagtttgggtaagcctaagaaaaaaaacataaccaagacagaggcacaatggttgttgtgcctcgaaaaccaaatagaagagctgcctgactggttcgcagctgaggagcccgtatttctggaagccaacatcaaacgtgacaagtatcacctagtgggggatatagctactatcaaagaaaaagctaaacaactgggggcaacagactccacaaagatatcaaaagaggttggagctaaagtatattctatgaagctgagtaactgggtgatacaagaagagaataaacagggcagcttagcccctttgtttgaagagctcctgcaacagtgcccacctgggggccagaataaaaccacacatatggtctcagcctaccaactggctcaagggaactggatgccagttggttgtcacgtgttcatggggaccatacccgccagaagaaccaagacccatccctatgaggcatacgtcaagctgagggagttggtagatgaatataagatgaagacgctatgtggcggttcaggcctaagtaagcacaacgaatgggtaattcgtaagatcaagcatcaagggaacctgaggaccaaacacatgttgaaccccgggaaggttgcagagcaactgcttagagaaggacacagacacaatgtatataataagactataggctcagtgatgacagcaactggtatcaggctggaaaagttacccgtggtcagggcccaaacagacacaaccaacttccatcaagcaataagggataaaatagacaaggaagagaacttacagactccaggcttacacaagaagttaatggaagtcttcaatgcattaaaaagacccgatcttgaggcctcttacgacgctgtggagtgggaggaattggagaaaggaataaataggaagggtgctgcaggtttctttgaacacaagaatataggagaggttctggattcagaaaaaaacaaggttgaagagatcatagacagtttgagaaaaggtaggagtatcaggtactatgaaactgcaatcccaaaaaacgagaagagggacgtcaatgatgactggactgccggtgactttgtagacgagaaaaagcccagggtgatacaataccctgaggctaaaaccaggttggccatcactaaagtaatgtacaagtgggtgaagcagaaaccagtagtcatacccggctatgaaggtaagacacctctgttccaaatctttgacaaagtgaagaaagaatgggaccaattccaaaatccagtggctgtgagctttgacaccaaagcgtgggacacccaggtgactacaggagacctggaactaataagggatatacagaagttctacttcaagaagaagtggcacaaattcattgacaccctaaccatgcacatgtcagaagtacccgtaattagtgccgacggggaggtgtacataaggaaggggcagagaggcagtggacaacctgatacgagcgcaggcaacagcatgttgaatgtattaacaatggtttatgccttctgtgaggccacgggagtgccctataagagttttgacagagtggcaaagatccacgtctgcggggatgatggtttcttgatcactgaaagggctcttggtgaaaaattttcgagtaaaggagtccagatcctatatgaagctggtaagccccaaaaaattacagaaggggataagatgaaagtggcctaccagtttgatgacatcgagttctgctcccatacaccagtgcaagtaaggtggtcagacaatacttccagttatatgccggggaggaacacgactacaatcctggcaaaaatggctacaaggttagattccagtggtgagaggggcactatagcatatgagaaggcggtggcgtttagcttcttattgatgtactcctggaacccactgatcagaaggatatgcttactggtgttgtcaactgaactgcaagtgagaccagggaagtcaaccacctattactatgaaggggacccaatttctgcttacaaggaggtcataggccataatctctttgaccttaaaagaacaagctttgagaaactagctaagttaaatcttagtatgtccacactcggggtatggaccagacacaccagcaaaagattactacaagactgtgtcaacgtcggcactaaagagggcaactggctggttaatgcagacagactggtgagtagcaagacaggaaataggtatatacctggagagggtcatacccagcaagggaaacattatgaagaactgatattggcaaggaagccgatcagcaattttgaagggactgatagatacaatttgggcccaatagtcaacgtagtgttgaggaggctgagagtcatgatgatggctcttataggaaggggggtgtaagcatggccggcccttaaccgggccctatcagtagaaccctgttgtaaataacactaacttattatttatttaaatactattatttatttatttatttatttattgaatgagcaaggattggtacaaactacctcatgttaccacactacactcattttaacagcactttagctggagggaaaatcctgacgtccatagttggactaaggtaatttcctaacggccc

SEQ ID NO: 2 - DNA, 12299 bp, Artificial Sequence
  GD18-wt
  misc_feature (12256)..(12256): n is a, c, g, or t
gtatacgaggttagttcgttctcgtgtacaatattggacaagaccaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggtgctgcagaggcccactaacaggctagtataaaaaatctctgctgtacatggcacatggagttgaatcattttgaactactatacaaaacaaataaacaaaaaccaataggagtggaggaaccggtgtatgacatcgcaggaagaccatttttcggggacccaagtgaggtacacccacaatcaacactgaagctgccacatgacagggggagaggtaatatcagaactacactgaagaacctacctaggaaaggcgactgtaggagtggaaaccacctaggaccagtcagtgggatatatgtaaaacccggccctgtcttctatcaggactacatgggccctgtctatcatagagctccattggagttttttgaagaggcccagctgtgtgaggtgactaagagaataggtagggtgacgggtagtgatgggaagctttaccacatatatgtgtgcattgacggttgcatactgctgaagttagccaaaaggggcacgcctagatccctaaagtggactaggaacttcaccgactgtccactatgggtcactagttgttctgatgatggcgcaggtggcagcaaggataagaaaccagacaggatgaacaagggtaaattaaaaatagccccaaaagagcatgagaaggacagcaaaaccaagccacctgatgccacgatcgtggtagagggagtaaaataccaagtgaaaaagaaaggcaaagtcaaagggaagaacactcaagatggcctataccataataagaacaaaccaccagagtccaggaagaaactagaaaaagccctactggcttgggcagtgataacaatcgtactgtaccagcctgtagcggccgagaatataactcagtggaacctgagtgacaatggcacaagcggtatccagcaagccatgtatcttagaggggttaataggagtctacatgggatctggcctgaaaaaatatgcaaaggggtccctactcacctggctactgatacggagctgacagagatacgagggatgatggacgccagcgagaggacaaactatacgtgttgcaggttgcagagacacgaatggaataaacatggatggtgtaactggtacaacatagacccctggattcaattaatgaacaggacccaagcaaatctgacagaaggccccccggataaggagtgcgccgtgacttgcaggtatgacaaaaatgctgacgttaacgtggtcacccaggccaggaatagaccaaccactctgaccggctgcaagaaaggaaaaaacttttcatttgcaggtacgattatagagggcccatgcaatttcaacgtctccgtggaggacatcctatatggggaccatgagtgtggcagcctgttccaggacacagctctgtacctattagatggaatgaccaacactatagagaaagccaggcagggtgcggcaagagttacatcctggcttgggaggcaactcagcaccacagggaagaagttggagagaggaagcaaaacctggttcggcgcctacgccctgtcaccttactgtaacgtaacaaggaaagtggggtacatatggtatacgaacaattgcactccggcatgtctcccaaaaaacacgaaaataataggtccaggaaaattcgataccaacgcggaagatgggaagatacttcatgaaatggggggccacctatcagaatttttgttgctttctttagtaatcctgtctgacttcgccccggagacggccagtacgctatacctaatcttacactatgcaatccctcagtcccatgaagagcctgaaggttgcgatacgaaccagctcaatctaacagtgggacttaggacagaagatgtggtaccatcatcagtttggaatatcggcaaatatgtgtgtgtcagaccagactggtggccgtatgaaactaaggtggctttgctgtttgaggaggcaggacaggttataaaactagctctacgggcactgagagatttaactagagtctggaacagtgcatcaactactgcgttcctcatctgcttgataaaaatattaagaggacaggttgtgcaaggtataatatggctgctgctggtaactggggcacaagggcggttgacctgtaaggaagactacaggtatgcgatatcatcaaccaatgagatagggccgcttggagctgaaggtctcactaccacctggaaagaatacaaccatggtttacagctggatgacgggactgtcagggccacttgcactgcagggtcctttaaagttatagcacttaatgtggttagtaggaggtacctggcatcattacacaagagggctttgcccacctcagtaacatttgaactcctatttgatgggaccagcccagtaattgaggagatgggagacgactttggatttgggctgtgcccttttgacacgatccctgtggtcaaagggaagtataacaccaccttattaaacggcagtgctttctatctagtctgccctataggatggacgggtgttatagagtgcacggcagtaagccccacaaccttgagaacagaagtggtgaagaccttcaagagagagaagcctttccctcacagagtggactgcgtgaccactatagtagaaaaagaagacctgttctattgcaggctggggggtaattggacatgcgtgaaaggtgacccggtgacctacacggggggccaagtaaaacaatgcaggtggtgcggtttcaacttcaaggagcctgatgggctcccacactaccccataggcaagtgcatcctagcaaatgagacgggatacagggtagtggactccacagactgcaacagagacggcgtcgtcatcggcactgaaggagagcatgagtgcttgatcggcaacaccaccgtcaaggtgcacgcgctggacggaagactggcccctatgccttgcagacccaaagaaatcgtatctagtgcgggacctgtaaggaaaacttcctgcacatttaactatacaaagactttgaggaacaagtactatgagcccagggacagctacttccagcaatacatgcttaagggcgagtatcaatactggtttgatctggacgtgacagaccaccacacagactacttcgccgagtttgttgtcttggtggtagtggcactattagggggaaggtacgtcctgtggctaatagtgacctatatagttctaacagagcaactcgctgctggtttacagctaggccagggcgaggtggtgctgatagggaacttaatcacccacatggacaacgaggtagtggtatacttcttactgctttacctaataataagagacgagcccataaagaaatggatactactgctgttccatgccatgaccaacaacccagttaagaccatgacagtagcattgctaatgataagcggggtagccaagggtgggaaaatagatggtggttggcagaggcagccggagaccaattttgacatccaattcgcactggcagtcataatagtcgtcgtgatgttgttggcaaagagagatccgactacttttcccctggtgatcactgtggcaaccttgagaacggctaagataaccagtggtttcagcacagatctagccatagccacagtgtcggcaactttgctaacctggacttatatcagtgactactacaaatacaagacttggctacagtacctcatcagcacggtgactggaatttttctgataagggtactgaagggggtcggcgagttggacctacacgccccgactctgccatcccacagaccccttttctacatccttgtgtacctcatctccactgccgtggtaacgagatggaacttggacgtagccggaatactgctacagtgcgccccaacacttctaatggtattcacgatgtgggctgacatcctcaccctaattctcatactgcccacctatgagttgacaaagttatattaccttaaggaagtgaagatagggactgaaagaggatggctgtggaaaactaattacaagagggtaaatgacatctatgaagtcgaccaagctggcgaaggggtttacctcttcccttcgaaacaaaagactggagctataacgagcaccgtgttgccgttgatcaaagccatactcattagctgcatcagtaacaagtggcaattcatatatttactgtacttgatatttgaggtgtcctactaccttcacaagaaaatcatagatgagatagccggcgggaccaacttcgtttcgagactagtggcggctttaattgaagttaattgggctctagacaatgaagaagtcaaaggcctaaagaagtttttcttgttgtctagtagggtcaaagagttggttatcaaacacaaagtgaggaatgaagtaatggtccgctggtttgaagatgaagagatctacgggatgccaaagctgatcggcctggttaaggcagcaacactgagcaaaaacaaacactgtattttgtgcaccgtctgtgaggacagagattggcggggagaaacttgcccgaaatgcgggcgttttggaccaccagtgatctgtggtatgaccctagctgacttcgaggaaaaacactataagaggattttcatcagggaggaccaatcagacgggccgctcagggaggagcatgcagggtacttgcagtacaaagctaggggtcaattgtttctgaggaatctcccagtgttggcaacaaaagtcaagatgcttctggttggcaacctagggacagaggttggggacctggagcaccttggctgggtgctcagagggcccgctgtctgcaagaaggttactgaacatgaaaagtgtgctacgtctataatggataagttgactgccttctttggtgtcatgccaaggggtaccacccccagagctcctgtaagatttcccacctcccttctaaagataagaagagggctggagacgggttgggcttatacacaccaaggcggtatcagctcagttgaccatgttacttgcgggaaagacctgctagtgtgtgacaccatgggtcggacaagggttgtctgccaatcgaataacaagatgactgacgagtctgaatacggagtcaaaaccgactcagggtgcccagagggagccaggtgctacgtgttcaaccctgaggcagttaatatatcaggcactaaaggagccatggtccacttacagaaaactggtggagaattcacctgtgtaacagcatcagggaccccagctttcttcgatctcaagaacctcaagggttggtcagggctaccgatatttgaagcatcgagtggaagggtagtcggaagggtcaaggtcgggaagaacgaagactctaaaccaaccaaactcatgagtgggatacaaacggtctctaaaagtgccactgatttgacggagatggtgaagaagataacaaccatgaacaggggggagttcagacaaataaccttggccacaggtgctggaaaaactacagagctacccaggtcagtcatagaagagatagggagacacaaaagggtgctggtattgatcccccttagggccgcagcagaatcagtataccaatacatgaggcagaaacacccgagtatagcattcaacctgaggataggggagatggaggaaggatacatggccacgggaataacctatgcttcttatggttacttttgccagatgccacaacccaagttgagagccgcaatggtagagtattcctacatctttctagacgagtaccattgcgccaccccggaacaattggctatcatggggaagatccacagattctcagagaacctgcgggtggtcgctatgacagcgacgccggcaggcacggtaacaactactgggcagaaacaccctatagaggagttcatagccccggaagtgatgaaaggagaagacttaggttcagagtacttagacatcgccggactaaagataccagtagaggagatgaagaacaacatgctagtttttgtaccaactaggaacatggcagtggaggcggcaaagaaattgaaagccaaaggatataactcaggctattactacagtggtgaggacccatccaacctgagggtagttacatcgcagtccccatacgtggtggtagcgaccaacgcaatagaatcaggcgtcactctcccagaccttgatgtggtcgttgacacaggactcaagtgtgagaaaagaatccgactgtcacccaagatgcctttcatagtgactggcctgaaaagaatggccgtcactattggggaacaagcccagagaagagggagggttggaagagtaaagcccgggagatactacaggagccaagaaactccagtcggctctaaggactaccactatgacttgctgcaagcccaaaggtacggtatagaagatgggataaatatcaccaaatccttcagagagatgaactacgattggagcctttatgaggaagatagcctgatgataacacaactggaaatcctcaacaatctgttgatatcagaggagctgccggtagcagtaaaaaatataatggctaggactgaccacccagaaccaattcaactagcgtataatagctacgagacacaggtaccagtgttgttcccaaagataaggaatggagaggtgactgacacttacgacaactacaccttcctcaatgcaagaaaattgggagatgatgtacccccctatgtgtatgccacagaagatgaggacttggcagtggaactgttaggcctagattggccagaccctggaaaccaaggcactgtggaggctggcagagcactaaaacaggtggtcggcctatcaacagccgagaatgccctgctagtggccttgtttggctacgtagggtaccaggccctctcaaaaaggcatatacctgtagtcacagacatatattcagttgaagatcacaggttggaagatacaacacacctacagtatgccccgaacgccatcaagacggaggggaaggagactgaattgaaggagttggctcaaggggatgtgcagagatgtgtggaagcagtggccaattatgcgagtgagggcatccaatttattaagtcacaggcactgaaggtgagagagaccccgacttacaaagagacaatgaatacagtggcagactatgtgaaaaagttcattgaggcactgtcagatagcaaggaagacatcttgaaatatgggctgtggggtgtacacacggccttgtataagagcattggtgccagactaggtcacgaaactgcgttcgcaactttagtcgtgaaatggttggcatttgggggggaatcagtaacagatcacataaagcaagcagccacagacttggttgtttactatattatcaacagacctcaattcccaggggacacggagacacaacaagaagggaggaaatttgtggccagcttactggtctcagccctagcaacctacacatacaaaagctggaactacaacaatctgtccaagatagttgaacctgctttggccactttgccctacgccgccaaagcccttaaattattcgcccctacccgactggagagcgtcgtcatattgagcaccgcaatctacaaaacatatctatcaatcaggcgaggcaaaagtgatggattgcttggtacaggggttagtgcggctatggagattatgtcacaaaatccagtatcagtgggcatagcagtcatgctgggggttggggctgtagcagcccataatgcaattgaagccagtgagcagaagagaacactactaatgaaagtctttgtgaaaaacttcttggaccaagcagccacggatgaactagtcaaagagagccctgagaaaataataatggccctgtttgaagcagtgcagacggtaggcaaccctctcagactggtgtaccacctatatggagttttttataaagggtgggaagcaaaagagttggcccaaaggacagccggcaggaacctcttcaccttaataatgttcgaggctgtggaactactgggagtagatagtgaagggaaaatccgccagctctcaagtaactacatattagagctcctgtacaagttccgtgacgacatcaaatctagtgtgagggagatagcaatcagttgggcccctgcccccttcagctgcgactggacaccaacagatgacagaataggacttccccatgacaattatctccagatggagacaagatgtccttgtggttacaggatgaaagcagtaaaaacctgtgccggggagttgagactcctggaagaggggggttcattcctctgcaggaataaattcggcagagggtcacggaattatagggtgacaaaatattatgatgacaacttatcagaaataaaaccagtgataagaatggaaggacacgtggaactgtatcataaaggggccactttcaaactggactttgacaacagtaaaacagtactggcaacagatagatgggaagtcgaccattccaccctgatcagggtgctcaagaggcacacaggggctggatttcaaggggcgtatctgggtgagaaacccaatcacaagcacctgatagagagagactgtgcaacgatcacaaaagacaaggtctgcttcatcaaaatgaagagggggtgcgcgttcacctatgacttatcactccacaaccttacccggctaatcgaattggtacataagaacaacctggatgacagagaaatccctgccgttacggtcacaacctggctggcctacacattcgtgaatgaagacatagggaccataaaaccagtcttcggggaaaaagtaacaccggagaagcaggaggaggtagccttgcagcctgcagtggtggtggacacaacagatgtggccgtgaccgtggtgggggaaacctccactatgaccacaggggagacccccacaatatttaccagcttaggttcggactctaagtttcaacaagtcctgaaactgggggtggatgagggtcaatatcccgggcccaggcagcagagagcaagtttgctcgaagctgtacaaggtgtagatgagaggccctcggtactaatattggggtctgataaggccacctccaatagggtgaagaccgcaaagaatgtgaagatattcaggagcagagaccccctggaactgagagagatgatgagaagagggaagatcctaatcatagccctgtgtaaggtggacaccgctctgctgaaatttgttgattacaaaggtaccttcctgaccagagagaccctagaggcattaagtctgggcaaacctaagaaaagagacataactaagacagaggcacgatggttgttgtgcctcgaaggtcaattagaagagctgcctgactggtttgcagccaaggagcccatattcttagaagccaacatcaaacgtgacaagtatcatctggtgggagacatagctacaatcaaagaaaaagccaagcaactgggggcaacagactccacaaagatatcaaaagaggttggcgcgaaagtgtattctatgaagttgagtaactgggtgatacaggaagagaataaacagggcagcttaacccccttgtttgaagagctcctgcaacagtgcccgcccgggggccagaataaaaccacacatatggcctcagcctaccaattggctcaggggaactggatgccagttggttgccacgtgttcatggggaccatacccgctagaagaaccaagactcatccctacgaggcatatgtcaagttgagggagttggtggaagaacataagatgaagacatcatgtggcggttcaggcctatgtaagcacaacgaatgggtaattcgtaagatcaagcatcaagggaacctgaggaccagacacatgctgaaccccggaaagattgctgagcaactggatagagaagggcacagacacaatgtgtacaacaagaccataggctcagtaatgacagcgactggcatcaggctggaaaagttacccgtggttagagcccagacagatacaactaatttccaccaagcaataagggataaaatagacaaggaagagaacctacagaccccaggcttacacaagaagttaatggaggtattcaatgcattaaaaagacctgagcttgaggcctcttacgacgccgtggagtgggaggaattggagagaggaataaataggaagggtgctgcagggttcttcgaacgcaagaatataggagaggtcctagattcagaaaaatataaggttgaagagatcatagacagtttgaagaaaggtaggagtattaaatattatgaaactgcaatcccaaagaacgaaaagagggatgtcaatgatgactggactgccggagactttgtggatgagaaaaaacccagggtgatacaatatcctgaggccaaaaccaggttagccatcactaaagtgatgtataagtgggtgaaacagaaaccagtagtcatacccggctatgaaggtaagacacctctgttccaaatttttgacaaagtgaaaaaagaatggaaccaattccaaaatccagtggcagtgagctttgacactaaagcatgggatacccaggtgactacaagagacctggaactaataagggatatacagaagttctatttcaagaagaagtggcacaaattcatcgacaccctaaccatgcacatgtcagaagtacccgtgattagtgccgacggggaggtgtatataaggaaagggcagagaggcagtgggcaacctgatacgagcgcaggcaacagtatgctgaatgtgttaacaatggtttatgccttctgtgaggccacgggggtaccctataaaagtttcgacagagtggcaaagatccacgtctgtggggatgatggcttcttgattactgaaagggctcttggtgaaaaatttgcaagtagaggagtccagatcctatatgaagccgggaagccccaaaaaatcactgaaggggacaatatgaaagtggcctatcagtttgacgacatcgagttctgctcccatacaccagtgcaggtaaggtggtcagacaacacctccagttacatgccggggaggaacacaaccacaatactggcaaaaatggctacaaggttggattccagtggtgagagaggcactatagcatatgagaaggcagtggcattcagtttcttgttgatgtactcctggaacccactgattagaaggatatgcttattggtgttatcaactgaaatgcaagtgagaccagggaagtcaaccacctattactatgaaggagacccaatatctgcttacaaggaggtcatcggccataatctctttgaccttaaaagaacaagtttcgagaagctagctaagctaaatcttagtatgtccacactcggggtatggaccaggcataccagcaaaagattactacaagactgtgtcaatgtcggcaccaaagagggcaactggctggttaacgcagacagactggtgagtagtaagacagggaataggtatatacctggagagggtcatacccaacaaggaaaacattatgaagagctggtattggcaagaaaaccgaccagtaattttgaagggactgatagatataatttgggcccaatagtcaacgtagtgttgaggaggctgagagtcatgatgatggctcttataggaaggggggtgtgagagcggccggctcttgaccgggccctatcagtataaccctgttgtaaataacactaacttattatttatctagacactactatttatttatttatttatttattgaatgagcaaggaatggtacgaactacctcatgttaccacactacactcattttaacagcactttagctgaggaaaaaantcctgacgtccatagttggactaaggtaatttcctaacggccc

SEQ ID NO: 3 - DNA, 12293 bp, Artificial Sequence
  QZ07-sdErnsH-KARD
gtatacgaggttagttcgttctcgtgtacaatattggacaaaacaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggcgctgcagaggcccactaacaggctagtataaaaatctctgctgtacatggcacatggagttgaatcattttgaactattgtataaaacaaacaaacaaaaaccaatgggagtggaggaaccggtgtatgacatcgcagggagaccattatttggggacccaagtgaggtacacccacaatcaacactgaagctaccacacgacaggggaagaggtaatatcagaactacactgaaggacctacctaggaaaggcgactgcaggagtggaaaccacctaggaccagttagtgggatatacgtaaagcctggccctgtcttctatcaggactacatgggccctgtctaccatagagccccattagagttttttggtgaggcccaattttgtgaggtgaccaagaggataggtagggtgacgggtagtgacggaaagctctaccacatatatgtgtgcatcgacggttgcatactgttgaagttagccaaaaggggcgcgcccagatccctaaagtggactaggaacttcaccgactgtccactgtgggtcactagttgttctgatgatggcacaggtgacagcaaggataaaaagccagacaggatgaacaagggtaaattaaagatagccccaaaagagcatgagaaggacagtaagaccaagccacctgatgctacaattgtggtagagggagtaaaataccaagtaaaaaagaaaggcaaagtcaaagggaagaacactcaagatggcctgtaccataataagaacaaaccaccagagtccaggaagaaactagaaaaagccctattggcctgggcagtgatagcaatagtgctgtaccagcctgtagcagccgagaatataacccaatggaacctgagtgacaacggcacaagcggcatccagcaagctatgtatctcagaggggtcaataggagcttgcatgggatctggcctgaaaaaatatgcaagggggtccctactcatctggccactgacacggaactgacagagatacgcgggatgatggacgccagtgagaggacgaactacacgtgttgtaggttgcagagacacgaatggaacaaaggctggtgcaactggtacaacatagacccctggattcagttaatgaacaggacccaagcaaatttgacagaaggccctccagataaagagtgtgccgtgacttgcaggtatgacaaaaataccgatgttaacgtggtcacccaggccaggaatagacctactactctgactggctgcaagaaagggaaaaatttttcatttgcaggtacggtcatagagggcccatgcaatttcaacgtatccgtggaggacatcttatatggcgaccatgagtgtggcagcctgttccaggacacggctctgtacctattagatggaatgaccaacactatagagaaagccaggcagggtgcggcaagagtcacatcttggctcgggagacaactcagaaccacagggaagaagttggagagaggaagcaaaacctggttcggtgcctatgccctgtcaccttactgcaatgtaacaaggaaaatagggtacatatggtacacgaacaattgcactccggcatgcctcccaaaaaacacaaaaataataggcccaggaaagtttgacaccaatgcagaagacgggaagattcttcatgaaatggggggccacctatcagaattcttgttgctttctctggtagtcctgtctgacttcgccccagaaacggccagtacattatacctaatcttacactatgcaatccctcagtcccgtgacgaacccgaggtttgcgatacaaaccagctcaacctaacagtgggacttaggacagaagatgtggtaccatcatcagtctggaatattggcaaatatgtgtgtgttagaccagattggtggccgtatgaaactaaggtggctctactgtttgaagaggcgggacaggtcataaagttagctctacgggcactgagggatttaactagagtctggaacagtgcatcaactactgcgttcctcatctgcttgataaaaatattaagaggacaggttgtgcaaggtataatatggctgctgctggtgactggggcacaagggcggttgtcctgcaaggaagactacaggtatgcgatatcaaagaccaatgagatagggccgctagcagctagggacctcaccaccacctggaaagaatataaccatggtttgcagctggatgacgggactgtcagggccatttgcactgcagggtcctttaaagttatagcacttaatgtggtcagtaggaggtacctggcatcattacacaagagggctttacccacttcagtaacatttgaactcctatttgatgggactagtccagcaattgaggagatgggagatgactttggatttgggctgtgcccttttgacacaacccccgtggtcaaagggaagtacaataccactttattaaacggtagtgctttctatctagtctgcccaataggatggacgggtgtcatagagtgcacggcagtaagccctacaaccttgagaacagaagtggtgaagaccttcaagagagagaagcctttcccacacagagtgggttgcgtgaccactgttgtagaaaaagaagacctgttctactgcaagtgggggggtaattggacatgtgtaaaaggcaacccggtgacctacatgggggggcaagtaaaacaatgcagatggtgcggttttgacttcaaggagcccgatgggctcccacactaccccataggcaagtgcatcctagcaaatgagacgggttacagggtagtggattccacagactgcaatagagatggcgtcgttatcagcactgaaggtgaacacgagtgcttgattggcaacaccaccgtcaaggtgcacgcgttggatggaagactgggccctatgccgtgcagacccaaagaaatcgtctctagcgcaggacctgtaaggaaaacttcctgcaccttcaactacacaaagacactaagaaacaagtactatgaacccagggacagctatttccagcaatatatgcttaagggcgagtaccaatactggtttgatctggacgtgaccgaccaccacacagactactttgccgaatttgttgtcttggtggtagtggcactattaggggggaggtacgtcctatggctaatagtgacctatgtaattttaacagagcaactcgctgccggtttacagctgggccaaggcgaggtagtactgatagggaacttaattacccacacggacaatgaggtagtggtatacttcttactgctctacctaataataagagacgagcctataaagaaatggatactactgctgtttcatgccatgaccaacaacccagttaagaccatgacagtagcattgctgatgatcagtggggtcgccaagggtgggaaaacagatggtggttggcagaggcagccggagaccaattttgacattcaactcgcactggcagttatagtagtcgttgtgatgttactggcaaagagagacccgactacttttcccctggtgatcacagtggcaaccttaagaacagctaagataaccaatggtttcagcacagatctagctatagccacagtgtcggcagctttgctaacctggacttatatcagtgactactacaaatataagacttggctacagtacctcatcagcacggtgactgggatcttcctaataagggtactgaagggaataggcgagctagacatgcacgccccaactttgccatctcacagaccccttttctacatcctcgtgtacctcatctccactgctgtggtgactagatggaacctggacgtagccggactgctgctacagtgtgtcccaacacttctaatggtgttcacgatgtgggctgacattctcaccctaatcctcgtgttacccacttatgaactggcaaagttgtattaccttaaggaagtgaagatcggaaccgaaagaggatggctgtggaaaactaactataagagggtaaatgacatctatgaagtcgaccaagctggcgagggggtctacctcttcccttcgaaacaaaagactagtgctataacaagcaccatgttgccattgatcaaagccatactcattagctgcatcagtaacaagtggcaactcatatatttactgtacctgatattcgaagtgtcctactaccttcacaagaaagtcatagatgaaatagctggtgggaccaacttcgtttcaaggctagtggcagccttaattgaagttaattgggccttcgacaatgaagaagttaaaggcctaaagaagttctttttgctgtccagtagagttaaagaattggtcatcaaacacaaagtgaggaatgaagtagtggcccgctggttcggagatgaagagatctatgggatgcccaagttgatcggcttagtcaaggcagcaactctaagtaaaaataaacactgtatcttgtgcaccgtctgcgaggacagagattggagaggagaaacctgcccaaaatgtgggcgtttcggaccaccagtgatctgtggtatgaccctagccgacttcgaggagaagcattataagaggatattcatcagggaggaccaatcagatggaccgctcagggaggagcgtgcagggtacttacagtacagagctaggggtcaactgtttctgaggaatctcccagtgttggcgacaaaagtcaagatgctcctggttggtaacctcgggacggaggttggggatttggagcaccttggctgggtgcttagagggcctgctgtctgcaagaaggttactgaacatgaaaagtgtgctacgtctataatggataagttgactgctttcttcggcgttatgccaagaggtaccacccccagagcccccgtgagatttcccacctccctcctaaagataagaagagggctagagacgggttgggcttacacacatcaaggtggcattagctcagttgaccatgtgacttgtgggaaagacttgctggtgtgtgacactatgggccggacaagggtcgtttgccaatcaaataataagatgactgacgaatccgaatacggggtcaaaaccgactcagggtgcccagaaggggccagatgttatgtgttcaaccctgaggcagttaacatatcaggcactaaaggggccatggtccacttacaaaaaactggtggggagttcacctgcgtaacagcatcaggaaccccggccttctttgatctcaagaacctcaagggctggtcagggctaccgatattcgaagcatcgagtggaagggtagtcggaagggtcaaggtcgggaagaacgatgactctaaaccaaccaaactcatgagtgggatacaaacggtctctaaaagtgccactgacctgacggagatggtaaagaagataacaactatgaacaggggagagttcagacagataaccttggccacaggtgctgggaaaactacagagctccccaggtcagtcatagaagagatagggagacacaaaagggtgttggtattgatccccttgagggccgcagcagaatcagtataccaatatatgaggcagaaacacccaagtatagcattcaacctgaggataggggagatgaaggaaggtcacatggccacggggataacctatgcctcctacggttacttttgccagatgccacaacctaagctgagggccgcaatggtagagtactcctacatctttctagatgagtaccattgtgccaccccagaacaactggctatcatggggaagatccacagattctcagagaacctgcgggtggtagctatgacagcgacaccagcagggacagtaacaactactgggcagaaacaccctatagaggagttcatagccccggaagtaatgaaaggagaagacttaggttcagagtacttagacattgccggactaaagataccagtagaggagatgaagaacaacatgctagtttttgtaccaactaggaatatggcggtagaggcggcaaagaaactgaaagctaaaggatacaactcaggctattactacagtggtgaggatccatctaacctgagggtagtcacatcgcagtccccatacgtggttgtagcgaccaacgcgatagaatcaggcgtcactctcccggaccttgacgtggtcgttgacacgggactcaagtgtgagaaaagaatccgattgtcacccaagatgcctttcatagtgactggcctaaaaagaatggctgttaccattggggaacaagcccagagaagagggagggttggaagagtaaagcccgggagatactacaggagccaagaaacccctgtcggctctaaggactaccactacgatttattgcaagcccagaggtacggtatagaagatgggataaatatcaccaaatccttcagagaaatgaactatgattggagcctctatgaggaagatagcctgatgataacacaattggaaatcctcaacaatctactgatatcagaggagctgccggtggcagtaaaaaatataatggctaggactgaccacccagaaccaattcaactagcatataatagctacgagacacaagtgcctgtgttgttcccaaaaataagaaatggagaggtgactgacacttacgatacttacaccttcatcaatgcaagaaaattgggagatgatgtacctccctacgtgtatgccacagaagatgaggacttggcagtggaactgttgggcctagattggccggaccctgggaaccaaggcactgtggaggccggcagagcactaaaacaggtggtcggtctatcaacagccgagaatgccctgttagtagccttgtttggctacgtagggtaccaggccctctcaaagaggcatatacctgtggtcacagacatatactcagttgaagatcacaggctagaggacaccacacacctacagtacgctccgaatgccatcaagacggaggggaaggagactgaattgaaggagttggctcagggggatgtgcagagatgcgtggaagcgatgaccaattacgcgagagggggcatccaatttatgaagtcacaggcactgcaggtgagagaaacccccacttataaagagacaatgaatactgtagcagactatgtgaaaaagtttatcgaggcactgtctgatagcaaggaagacatcttgaaatatgggctgtggggcgtacatacggccttgtacaagagcattggtgccaggcttggtcacgaaaccgcgttcgcaaccctagccgtgaaatggctggcatttgggggggaatcaatagcagatcatataaaacaagcggctacagacttggtcgtctactatatcatcaacagacctcagttcccaggagacacagagacacaacaggaggggagaaattttgtggccagcctactggtctcagccctagcaacttacacatataaaagctggaactataataacctgtccaagatagtagaaccggctttggccaccctgccctatgccgctaaagcccttaaattatttgcccctaccaggctagagagcgtcgtcatattgagcaccgcaatctacaaaacatacctatcaatcaggcgaggcaaaagcgatggattgctaggtacaggggttagtgcggctatggagattatgtcacaaaatccggtatcagtgggcatagcagttatgctgggggttggggctgtagcagcacacaacgcaattgaagccagtgagcagaagagaacactacttatgaaagtttttgtaaagaacttcttggaccaagcagccacagatgaactagtcaaagagagccctgagaaaataataatggccttgtttgaagcagtgcaaacagtaggcaatcctcttaggctagtgtaccacctttacggggttttttataaagggtgggaagcaaaagagttggcccaaaggacagccggcaggaatcttttcaccttgataatgtttgaggccgtggaactattgggagtagatagcgaaggaaaaatccgccaactatcgagtaattacatattagagctcctgtataagttccgtgacagtatcaaatctagtgtgagggagatagcaatcagctgggcccctgccccctttagttgcgattggacaccaacagatgacagaatagggcttccccataacaattacctccaaatggagacaagatgcccttgtggctacaggatgaaagcagtaaaaacctgtgctggggagttgagacttctggaagagggaggttcattcctctgcaggaataaattcggtagagggtcacggaactatagggtgacaaaatactatgatgacaatttatcagaaataaaaccagtgataagaatggaaggacatgtggaactatattataagggggccactatcaaactggactttaacaacagtaaaacagtactggcaaccgacaaatgggaggttgaccattccaccctggtcagggcgctcaagaggcacacaggggctgggtaccaaggagcgtatatgggtgagaaacctaaccacaaacatctgatagagagagactgtgcaacgatcacaaaagacaaggtttacttcatcaagatgaagagggggtgtgcgttcacttatgacttgtccctccacaacctcacccggttaatcgaattggtacacaaaaacgacctggaagatagagaaatccctgctgttacggttacaacctggctggcctacacatttgtgaatgaagacatagggaccataaaaccagtttttggggaaaaagtaacaccggaaaaacaggaggaggtagccttgcagcctgctgtggtggtggacacaacagatgtggccgtgaccgtggtgggggaaacctctactatgactacaggggagaccccaacggcattcaccagcttaggttcagactctaaagttcaacaagttctgaagttaggggtggatgagggtcaataccccgggcccagtcagcagagagcaagcttgctcgacgctatacaaggcgtggatgaaaggccctcggtattgatattgggatctgataaggccacctccaatagggtgaagaccgcaaagaatgtcaagatattcaggagcagagaccccctggaactgagagaaatgatgagaagagggaagatcctggtcatagccctatgtaaggtcgacactgctctactgaaatttgttgattacaaaggcaccttcctgaccagagaaaccctagaggcattaagtttgggtaagcctaagaaaaaaaacataaccaagacagaggcacaatggttgttgtgcctcgaaaaccaaatagaagagctgcctgactggttcgcagctgaggagcccgtatttctggaagccaacatcaaacgtgacaagtatcacctagtgggggatatagctactatcaaagaaaaagctaaacaactgggggcaacagactccacaaagatatcaaaagaggttggagctaaagtatattctatgaagctgagtaactgggtgatacaagaagagaataaacagggcagcttagcccctttgtttgaagagctcctgcaacagtgcccacctgggggccagaataaaaccacacatatggtctcagcctaccaactggctcaagggaactggatgccagttggttgtcacgtgttcatggggaccatacccgccagaagaaccaagacccatccctatgaggcatacgtcaagctgagggagttggtagatgaatataagatgaagacgctatgtggcggttcaggcctaagtaagcacaacgaatgggtaattcgtaagatcaagcatcaagggaacctgaggaccaaacacatgttgaaccccgggaaggttgcagagcaactgcttagagaaggacacagacacaatgtatataataagactataggctcagtgatgacagcaactggtatcaggctggaaaagttacccgtggtcagggcccaaacagacacaaccaacttccatcaagcaataagggataaaatagacaaggaagagaacttacagactccaggcttacacaagaagttaatggaagtcttcaatgcattaaaaagacccgatcttgaggcctcttacgacgctgtggagtgggaggaattggagaaaggaataaataggaagggtgctgcaggtttctttgaacacaagaatataggagaggttctggattcagaaaaaaacaaggttgaagagatcatagacagtttgagaaaaggtaggagtatcaggtactatgaaactgcaatcccaaaaaacgagaagagggacgtcaatgatgactggactgccggtgactttgtagacgagaaaaagcccagggtgatacaataccctgaggctaaaaccaggttggccatcactaaagtaatgtacaagtgggtgaagcagaaaccagtagtcatacccggctatgaaggtaagacacctctgttccaaatctttgacaaagtgaagaaagaatgggaccaattccaaaatccagtggctgtgagctttgacaccaaagcgtgggacacccaggtgactacaggagacctggaactaataagggatatacagaagttctacttcaagaagaagtggcacaaattcattgacaccctaaccatgcacatgtcagaagtacccgtaattagtgccgacggggaggtgtacataaggaaggggcagagaggcagtggacaacctgatacgagcgcaggcaacagcatgttgaatgtattaacaatggtttatgccttctgtgaggccacgggagtgccctataagagttttgacagagtggcaaagatccacgtctgcggggatgatggtttcttgatcactgaaagggctcttggtgaaaaattttcgagtaaaggagtccagatcctatatgaagctggtaagccccaaaaaattacagaaggggataagatgaaagtggcctaccagtttgatgacatcgagttctgctcccatacaccagtgcaagtaaggtggtcagacaatacttccagttatatgccggggaggaacacgactacaatcctggcaaaaatggctacaaggttagattccagtggtgagaggggcactatagcatatgagaaggcggtggcgtttagcttcttattgatgtactcctggaacccactgatcagaaggatatgcttactggtgttgtcaactgaactgcaagtgagaccagggaagtcaaccacctattactatgaaggggacccaatttctgcttacaaggaggtcataggccataatctctttgaccttaaaagaacaagctttgagaaactagctaagttaaatcttagtatgtccacactcggggtatggaccagacacaccagcaaaagattactacaagactgtgtcaacgtcggcactaaagagggcaactggctggttaatgcagacagactggtgagtagcaagacaggaaataggtatatacctggagagggtcatacccagcaagggaaacattatgaagaactgatattggcaaggaagccgatcagcaattttgaagggactgatagatacaatttgggcccaatagtcaacgtagtgttgaggaggctgagagtcatgatgatggctcttataggaaggggggtgtaagcatggccggcccttaaccgggccctatcagtagaaccctgttgtaaataacactaacttattatttatttaaatactattatttatttatttatttatttattgaatgagcaaggattggtacaaactacctcatgttaccacactacactcattttaacagcactttagctggagggaaaatcctgacgtccatagttggactaaggtaatttcctaacggccc

SEQ ID NO: 4 - DNA, 12290 bp, Artificial Sequence
  QZ07-ddErnsHC-KARD
gtatacgaggttagttcgttctcgtgtacaatattggacaaaacaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggcgctgcagaggcccactaacaggctagtataaaaatctctgctgtacatggcacatggagttgaatcattttgaactattgtataaaacaaacaaacaaaaaccaatgggagtggaggaaccggtgtatgacatcgcagggagaccattatttggggacccaagtgaggtacacccacaatcaacactgaagctaccacacgacaggggaagaggtaatatcagaactacactgaaggacctacctaggaaaggcgactgcaggagtggaaaccacctaggaccagttagtgggatatacgtaaagcctggccctgtcttctatcaggactacatgggccctgtctaccatagagccccattagagttttttggtgaggcccaattttgtgaggtgaccaagaggataggtagggtgacgggtagtgacggaaagctctaccacatatatgtgtgcatcgacggttgcatactgttgaagttagccaaaaggggcgcgcccagatccctaaagtggactaggaacttcaccgactgtccactgtgggtcactagttgttctgatgatggcacaggtgacagcaaggataaaaagccagacaggatgaacaagggtaaattaaagatagccccaaaagagcatgagaaggacagtaagaccaagccacctgatgctacaattgtggtagagggagtaaaataccaagtaaaaaagaaaggcaaagtcaaagggaagaacactcaagatggcctgtaccataataagaacaaaccaccagagtccaggaagaaactagaaaaagccctattggcctgggcagtgatagcaatagtgctgtaccagcctgtagcagccgagaatataacccaatggaacctgagtgacaacggcacaagcggcatccagcaagctatgtatctcagaggggtcaataggagcttgcatgggatctggcctgaaaaaatatgcaagggggtccctactcatctggccactgacacggaactgacagagatacgcgggatgatggacgccagtgagaggacgaactacacgtgttgtaggttgcagagacacgaatggaacaaaggctggtgcaactggtacaacatagacccctggattcagttaatgaacaggacccaagcaaatttgacagaaggccctccagataaagagtgtgccgtgacttgcaggtatgacaaaaataccgatgttaacgtggtcacccaggccaggaatagacctactactctgactggctgcaagaaagggaaaaatttttcatttgcaggtacggtcatagagggcccatgcaatttcaacgtatccgtggaggacatcttatatggcgaccatgagggcagcctgttccaggacacggctctgtacctattagatggaatgaccaacactatagagaaagccaggcagggtgcggcaagagtcacatcttggctcgggagacaactcagaaccacagggaagaagttggagagaggaagcaaaacctggttcggtgcctatgccctgtcaccttactgcaatgtaacaaggaaaatagggtacatatggtacacgaacaattgcactccggcatgcctcccaaaaaacacaaaaataataggcccaggaaagtttgacaccaatgcagaagacgggaagattcttcatgaaatggggggccacctatcagaattcttgttgctttctctggtagtcctgtctgacttcgccccagaaacggccagtacattatacctaatcttacactatgcaatccctcagtcccgtgacgaacccgaggtttgcgatacaaaccagctcaacctaacagtgggacttaggacagaagatgtggtaccatcatcagtctggaatattggcaaatatgtgtgtgttagaccagattggtggccgtatgaaactaaggtggctctactgtttgaagaggcgggacaggtcataaagttagctctacgggcactgagggatttaactagagtctggaacagtgcatcaactactgcgttcctcatctgcttgataaaaatattaagaggacaggttgtgcaaggtataatatggctgctgctggtgactggggcacaagggcggttgtcctgcaaggaagactacaggtatgcgatatcaaagaccaatgagatagggccgctagcagctagggacctcaccaccacctggaaagaatataaccatggtttgcagctggatgacgggactgtcagggccatttgcactgcagggtcctttaaagttatagcacttaatgtggtcagtaggaggtacctggcatcattacacaagagggctttacccacttcagtaacatttgaactcctatttgatgggactagtccagcaattgaggagatgggagatgactttggatttgggctgtgcccttttgacacaacccccgtggtcaaagggaagtacaataccactttattaaacggtagtgctttctatctagtctgcccaataggatggacgggtgtcatagagtgcacggcagtaagccctacaaccttgagaacagaagtggtgaagaccttcaagagagagaagcctttcccacacagagtgggttgcgtgaccactgttgtagaaaaagaagacctgttctactgcaagtgggggggtaattggacatgtgtaaaaggcaacccggtgacctacatgggggggcaagtaaaacaatgcagatggtgcggttttgacttcaaggagcccgatgggctcccacactaccccataggcaagtgcatcctagcaaatgagacgggttacagggtagtggattccacagactgcaatagagatggcgtcgttatcagcactgaaggtgaacacgagtgcttgattggcaacaccaccgtcaaggtgcacgcgttggatggaagactgggccctatgccgtgcagacccaaagaaatcgtctctagcgcaggacctgtaaggaaaacttcctgcaccttcaactacacaaagacactaagaaacaagtactatgaacccagggacagctatttccagcaatatatgcttaagggcgagtaccaatactggtttgatctggacgtgaccgaccaccacacagactactttgccgaatttgttgtcttggtggtagtggcactattaggggggaggtacgtcctatggctaatagtgacctatgtaattttaacagagcaactcgctgccggtttacagctgggccaaggcgaggtagtactgatagggaacttaattacccacacggacaatgaggtagtggtatacttcttactgctctacctaataataagagacgagcctataaagaaatggatactactgctgtttcatgccatgaccaacaacccagttaagaccatgacagtagcattgctgatgatcagtggggtcgccaagggtgggaaaacagatggtggttggcagaggcagccggagaccaattttgacattcaactcgcactggcagttatagtagtcgttgtgatgttactggcaaagagagacccgactacttttcccctggtgatcacagtggcaaccttaagaacagctaagataaccaatggtttcagcacagatctagctatagccacagtgtcggcagctttgctaacctggacttatatcagtgactactacaaatataagacttggctacagtacctcatcagcacggtgactgggatcttcctaataagggtactgaagggaataggcgagctagacatgcacgccccaactttgccatctcacagaccccttttctacatcctcgtgtacctcatctccactgctgtggtgactagatggaacctggacgtagccggactgctgctacagtgtgtcccaacacttctaatggtgttcacgatgtgggctgacattctcaccctaatcctcgtgttacccacttatgaactggcaaagttgtattaccttaaggaagtgaagatcggaaccgaaagaggatggctgtggaaaactaactataagagggtaaatgacatctatgaagtcgaccaagctggcgagggggtctacctcttcccttcgaaacaaaagactagtgctataacaagcaccatgttgccattgatcaaagccatactcattagctgcatcagtaacaagtggcaactcatatatttactgtacctgatattcgaagtgtcctactaccttcacaagaaagtcatagatgaaatagctggtgggaccaacttcgtttcaaggctagtggcagccttaattgaagttaattgggccttcgacaatgaagaagttaaaggcctaaagaagttctttttgctgtccagtagagttaaagaattggtcatcaaacacaaagtgaggaatgaagtagtggcccgctggttcggagatgaagagatctatgggatgcccaagttgatcggcttagtcaaggcagcaactctaagtaaaaataaacactgtatcttgtgcaccgtctgcgaggacagagattggagaggagaaacctgcccaaaatgtgggcgtttcggaccaccagtgatctgtggtatgaccctagccgacttcgaggagaagcattataagaggatattcatcagggaggaccaatcagatggaccgctcagggaggagcgtgcagggtacttacagtacagagctaggggtcaactgtttctgaggaatctcccagtgttggcgacaaaagtcaagatgctcctggttggtaacctcgggacggaggttggggatttggagcaccttggctgggtgcttagagggcctgctgtctgcaagaaggttactgaacatgaaaagtgtgctacgtctataatggataagttgactgctttcttcggcgttatgccaagaggtaccacccccagagcccccgtgagatttcccacctccctcctaaagataagaagagggctagagacgggttgggcttacacacatcaaggtggcattagctcagttgaccatgtgacttgtgggaaagacttgctggtgtgtgacactatgggccggacaagggtcgtttgccaatcaaataataagatgactgacgaatccgaatacggggtcaaaaccgactcagggtgcccagaaggggccagatgttatgtgttcaaccctgaggcagttaacatatcaggcactaaaggggccatggtccacttacaaaaaactggtggggagttcacctgcgtaacagcatcaggaaccccggccttctttgatctcaagaacctcaagggctggtcagggctaccgatattcgaagcatcgagtggaagggtagtcggaagggtcaaggtcgggaagaacgatgactctaaaccaaccaaactcatgagtgggatacaaacggtctctaaaagtgccactgacctgacggagatggtaaagaagataacaactatgaacaggggagagttcagacagataaccttggccacaggtgctgggaaaactacagagctccccaggtcagtcatagaagagatagggagacacaaaagggtgttggtattgatccccttgagggccgcagcagaatcagtataccaatatatgaggcagaaacacccaagtatagcattcaacctgaggataggggagatgaaggaaggtcacatggccacggggataacctatgcctcctacggttacttttgccagatgccacaacctaagctgagggccgcaatggtagagtactcctacatctttctagatgagtaccattgtgccaccccagaacaactggctatcatggggaagatccacagattctcagagaacctgcgggtggtagctatgacagcgacaccagcagggacagtaacaactactgggcagaaacaccctatagaggagttcatagccccggaagtaatgaaaggagaagacttaggttcagagtacttagacattgccggactaaagataccagtagaggagatgaagaacaacatgctagtttttgtaccaactaggaatatggcggtagaggcggcaaagaaactgaaagctaaaggatacaactcaggctattactacagtggtgaggatccatctaacctgagggtagtcacatcgcagtccccatacgtggttgtagcgaccaacgcgatagaatcaggcgtcactctcccggaccttgacgtggtcgttgacacgggactcaagtgtgagaaaagaatccgattgtcacccaagatgcctttcatagtgactggcctaaaaagaatggctgttaccattggggaacaagcccagagaagagggagggttggaagagtaaagcccgggagatactacaggagccaagaaacccctgtcggctctaaggactaccactacgatttattgcaagcccagaggtacggtatagaagatgggataaatatcaccaaatccttcagagaaatgaactatgattggagcctctatgaggaagatagcctgatgataacacaattggaaatcctcaacaatctactgatatcagaggagctgccggtggcagtaaaaaatataatggctaggactgaccacccagaaccaattcaactagcatataatagctacgagacacaagtgcctgtgttgttcccaaaaataagaaatggagaggtgactgacacttacgatacttacaccttcatcaatgcaagaaaattgggagatgatgtacctccctacgtgtatgccacagaagatgaggacttggcagtggaactgttgggcctagattggccggaccctgggaaccaaggcactgtggaggccggcagagcactaaaacaggtggtcggtctatcaacagccgagaatgccctgttagtagccttgtttggctacgtagggtaccaggccctctcaaagaggcatatacctgtggtcacagacatatactcagttgaagatcacaggctagaggacaccacacacctacagtacgctccgaatgccatcaagacggaggggaaggagactgaattgaaggagttggctcagggggatgtgcagagatgcgtggaagcgatgaccaattacgcgagagggggcatccaatttatgaagtcacaggcactgcaggtgagagaaacccccacttataaagagacaatgaatactgtagcagactatgtgaaaaagtttatcgaggcactgtctgatagcaaggaagacatcttgaaatatgggctgtggggcgtacatacggccttgtacaagagcattggtgccaggcttggtcacgaaaccgcgttcgcaaccctagccgtgaaatggctggcatttgggggggaatcaatagcagatcatataaaacaagcggctacagacttggtcgtctactatatcatcaacagacctcagttcccaggagacacagagacacaacaggaggggagaaattttgtggccagcctactggtctcagccctagcaacttacacatataaaagctggaactataataacctgtccaagatagtagaaccggctttggccaccctgccctatgccgctaaagcccttaaattatttgcccctaccaggctagagagcgtcgtcatattgagcaccgcaatctacaaaacatacctatcaatcaggcgaggcaaaagcgatggattgctaggtacaggggttagtgcggctatggagattatgtcacaaaatccggtatcagtgggcatagcagttatgctgggggttggggctgtagcagcacacaacgcaattgaagccagtgagcagaagagaacactacttatgaaagtttttgtaaagaacttcttggaccaagcagccacagatgaactagtcaaagagagccctgagaaaataataatggccttgtttgaagcagtgcaaacagtaggcaatcctcttaggctagtgtaccacctttacggggttttttataaagggtgggaagcaaaagagttggcccaaaggacagccggcaggaatcttttcaccttgataatgtttgaggccgtggaactattgggagtagatagcgaaggaaaaatccgccaactatcgagtaattacatattagagctcctgtataagttccgtgacagtatcaaatctagtgtgagggagatagcaatcagctgggcccctgccccctttagttgcgattggacaccaacagatgacagaatagggcttccccataacaattacctccaaatggagacaagatgcccttgtggctacaggatgaaagcagtaaaaacctgtgctggggagttgagacttctggaagagggaggttcattcctctgcaggaataaattcggtagagggtcacggaactatagggtgacaaaatactatgatgacaatttatcagaaataaaaccagtgataagaatggaaggacatgtggaactatattataagggggccactatcaaactggactttaacaacagtaaaacagtactggcaaccgacaaatgggaggttgaccattccaccctggtcagggcgctcaagaggcacacaggggctgggtaccaaggagcgtatatgggtgagaaacctaaccacaaacatctgatagagagagactgtgcaacgatcacaaaagacaaggtttacttcatcaagatgaagagggggtgtgcgttcacttatgacttgtccctccacaacctcacccggttaatcgaattggtacacaaaaacgacctggaagatagagaaatccctgctgttacggttacaacctggctggcctacacatttgtgaatgaagacatagggaccataaaaccagtttttggggaaaaagtaacaccggaaaaacaggaggaggtagccttgcagcctgctgtggtggtggacacaacagatgtggccgtgaccgtggtgggggaaacctctactatgactacaggggagaccccaacggcattcaccagcttaggttcagactctaaagttcaacaagttctgaagttaggggtggatgagggtcaataccccgggcccagtcagcagagagcaagcttgctcgacgctatacaaggcgtggatgaaaggccctcggtattgatattgggatctgataaggccacctccaatagggtgaagaccgcaaagaatgtcaagatattcaggagcagagaccccctggaactgagagaaatgatgagaagagggaagatcctggtcatagccctatgtaaggtcgacactgctctactgaaatttgttgattacaaaggcaccttcctgaccagagaaaccctagaggcattaagtttgggtaagcctaagaaaaaaaacataaccaagacagaggcacaatggttgttgtgcctcgaaaaccaaatagaagagctgcctgactggttcgcagctgaggagcccgtatttctggaagccaacatcaaacgtgacaagtatcacctagtgggggatatagctactatcaaagaaaaagctaaacaactgggggcaacagactccacaaagatatcaaaagaggttggagctaaagtatattctatgaagctgagtaactgggtgatacaagaagagaataaacagggcagcttagcccctttgtttgaagagctcctgcaacagtgcccacctgggggccagaataaaaccacacatatggtctcagcctaccaactggctcaagggaactggatgccagttggttgtcacgtgttcatggggaccatacccgccagaagaaccaagacccatccctatgaggcatacgtcaagctgagggagttggtagatgaatataagatgaagacgctatgtggcggttcaggcctaagtaagcacaacgaatgggtaattcgtaagatcaagcatcaagggaacctgaggaccaaacacatgttgaaccccgggaaggttgcagagcaactgcttagagaaggacacagacacaatgtatataataagactataggctcagtgatgacagcaactggtatcaggctggaaaagttacccgtggtcagggcccaaacagacacaaccaacttccatcaagcaataagggataaaatagacaaggaagagaacttacagactccaggcttacacaagaagttaatggaagtcttcaatgcattaaaaagacccgatcttgaggcctcttacgacgctgtggagtgggaggaattggagaaaggaataaataggaagggtgctgcaggtttctttgaacacaagaatataggagaggttctggattcagaaaaaaacaaggttgaagagatcatagacagtttgagaaaaggtaggagtatcaggtactatgaaactgcaatcccaaaaaacgagaagagggacgtcaatgatgactggactgccggtgactttgtagacgagaaaaagcccagggtgatacaataccctgaggctaaaaccaggttggccatcactaaagtaatgtacaagtgggtgaagcagaaaccagtagtcatacccggctatgaaggtaagacacctctgttccaaatctttgacaaagtgaagaaagaatgggaccaattccaaaatccagtggctgtgagctttgacaccaaagcgtgggacacccaggtgactacaggagacctggaactaataagggatatacagaagttctacttcaagaagaagtggcacaaattcattgacaccctaaccatgcacatgtcagaagtacccgtaattagtgccgacggggaggtgtacataaggaaggggcagagaggcagtggacaacctgatacgagcgcaggcaacagcatgttgaatgtattaacaatggtttatgccttctgtgaggccacgggagtgccctataagagttttgacagagtggcaaagatccacgtctgcggggatgatggtttcttgatcactgaaagggctcttggtgaaaaattttcgagtaaaggagtccagatcctatatgaagctggtaagccccaaaaaattacagaaggggataagatgaaagtggcctaccagtttgatgacatcgagttctgctcccatacaccagtgcaagtaaggtggtcagacaatacttccagttatatgccggggaggaacacgactacaatcctggcaaaaatggctacaaggttagattccagtggtgagaggggcactatagcatatgagaaggcggtggcgtttagcttcttattgatgtactcctggaacccactgatcagaaggatatgcttactggtgttgtcaactgaactgcaagtgagaccagggaagtcaaccacctattactatgaaggggacccaatttctgcttacaaggaggtcataggccataatctctttgaccttaaaagaacaagctttgagaaactagctaagttaaatcttagtatgtccacactcggggtatggaccagacacaccagcaaaagattactacaagactgtgtcaacgtcggcactaaagagggcaactggctggttaatgcagacagactggtgagtagcaagacaggaaataggtatatacctggagagggtcatacccagcaagggaaacattatgaagaactgatattggcaaggaagccgatcagcaattttgaagggactgatagatacaatttgggcccaatagtcaacgtagtgttgaggaggctgagagtcatgatgatggctcttataggaaggggggtgtaagcatggccggcccttaaccgggccctatcagtagaaccctgttgtaaataacactaacttattatttatttaaatactattatttatttatttatttatttattgaatgagcaaggattggtacaaactacctcatgttaccacactacactcattttaacagcactttagctggagggaaaatcctgacgtccatagttggactaaggtaatttcctaacggccc

SEQ ID NO: 5 - DNA, 12293 bp, Artificial Sequence
  GD18-ddErnsHC-KARD
  misc_feature (12250)..(12250): n is a, c, g, or t
gtatacgaggttagttcgttctcgtgtacaatattggacaagaccaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggtgctgcagaggcccactaacaggctagtataaaaaatctctgctgtacatggcacatggagttgaatcattttgaactactatacaaaacaaataaacaaaaaccaataggagtggaggaaccggtgtatgacatcacaggaagaccatttttcggggacccaagtgaggtacacccacaatcaacactgaagctgccacatgacagggggagaggtaatatcagaactacactgaagaacctacctaggaaaggcgactgtaggagtggaaaccacctaggaccagtcagtgggatatatgtaaaacccggccctgtcttctatcaggactacatgggccctgtctatcatagagctccattggagttttttgaagaggcccagctgtgtgaggtgactaagagaataggtagggtgacgggtagtgatgggaagctttaccacatatatgtgtgcattgacggttgcatactgctgaagttagccaaaaggggcacgcctagatccctaaagtggactaggaacttcaccgactgtccactatgggtcactagttgttctgatgatggcgcaggtggcagcaaggataagaaaccagacaggatgaacaagggtaaattaaaaatagccccaaaagagcatgagaaggacagcaaaaccaagccacctgatgccacgatcgtggtagagggagtaaaataccaagtgaaaaagaaaggcaaagtcaaagggaagaacactcaagatggcctataccataataagaacaaaccaccagagtccaggaagaaactagaaaaagccctactggcttgggcagtgataacaatcgtactgtaccagcctgtagcggccgagaatataactcagtggaacctgagtgacaatggcacaagcggtatccagcaagccatgtatcttagaggggttaataggagtctacatgggatctggcctgaaaaaatatgcaaaggggtccctactcacctggctactgatacggagctgacagagatacgagggatgatggacgccagcgagaggacaaactatacgtgttgcaggttgcagagacacgaatggaataaaggatggtgtaactggtacaacatagacccctggattcaattaatgaacaggacccaagcaaatctgacagaaggccccccggataaggagtgcgccgtgacttgcaggtatgacaaaaatgctgacgttaacgtggtcacccaggccaggaatagaccaaccactctgaccggctgcaagaaaggaaaaaacttttcatttgcaggtacgattatagagggcccatgcaatttcaacgtctccgtggaggacatcctatatggggaccatgagggcagcctgttccaggacacagctctgtacctattagatggaatgaccaacactatagagaaagccaggcagggtgcggcaagagttacatcctggcttgggaggcaactcagaaccacagggaagaagttggagagaggaagcaaaacctggttcggcgcctacgccctgtcaccttactgtaacgtaacaaggaaagtggggtacatatggtatacgaacaattgcactccggcatgtctcccaaaaaacacgaaaataataggtccaggaaaattcgataccaacgcggaagatgggaagatacttcatgaaatggggggccacctatcagaatttttgttgctttctttagtaatcctgtctgacttcgccccggagacggccagtacgctatacctaatcttacactatgcaatccctcagtcccatgaagagcctgagggttgcgatacgaaccagctcaatctaacagtgggacttaggacagaagatgtggtaccatcatcagtttggaatatcggcaaatatgtgtgtgtcagaccagactggtggccgtatgaaactaaggtggctttgctgtttgaggaggcaggacaggttataaaactagctctacgggcactgagagatttaactagagtctggaacagtgcatcaactactgcgttcctcatctgcttgataaaaatattaagaggacaggttgtgcaaggtataatatggctgctgctggtaactggggcacaagggcggttgacctgtaaggaagactacaggtatgcgatatcaaagaccaatgagatagggccgcttgccgctagggacctcactaccacctggaaagaatacaaccatggtttacagctggatgacgggactgtcagggccacttgcactgcagggtcctttaaagttatagcacttaatgtggttagtaggaggtacctggcatcattacacaagagggctttgcccacctcagtaacatttgaactcctatttgatgggaccagcccagtaattgaggagatgggagacgactttggatttgggctgtgcccttttgacacgatccctgtggtcaaagggaagtataacaccaccttattaaacggcagtgctttctatctagtctgccctataggatggacgggtgttatagagtgcacggcagtaagccccacaaccttgagaacagaagtggtgaagaccttcaagagagagaagcctttccctcacagagtggactgcgtgaccactatagtagaaaaagaagacctgttctattgcaggctggggggtaattggacatgcgtgaaaggtgacccggtgacctacacggggggccaagtaaaacaatgcaggtggtgcggtttcaacttcaaggagcctgatgggctcccacactaccccataggcaagtgcatcctagcaaatgagacgggatacagggtagtggactccacagactgcaacagagacggcgtcgtcatcggcactgaaggagagcatgagtgcttgatcggcaacaccaccgtcaaggtgcacgcgctggacggaagactggcccctatgccttgcagacccaaagaaatcgtatctagtgcgggacctgtaaggaaaacttcctgcacatttaactatacaaagactttgaggaacaagtactatgagcccagggacagctacttccagcaatacatgcttaagggcgagtatcaatactggtttgatctggacgtgacagaccaccacacagactacttcgccgagtttgttgtcttggtggtagtggcactattagggggaaggtacgtcctgtggctaatagtgacctatatagttctaacagagcaactcgctgctggtttacagctaggccagggcgaggtggtgctgatagggaacttaatcacccacatggacaacgaggtagtggtatacttcttactgctttacctaataataagagacgagcccataaagaaatggatactactgctgttccatgccatgaccaacaacccagttaagaccatgacagtagcattgctaatgataagcggggtagccaagggtgggaaaatagatggtggttggcagaggcagccggagaccaattttgacatccaattcgcactggcagtcataatagtcgtcgtgatgttgttggcaaagagagatccgactacttttcccctggtgatcactgtggcaaccttgagaacggctaagataaccagtggtttcagcacagatctagccatagccacagtgtcggcaactttgctaacctggacttatatcagtgactactacaaatacaagacttggctacagtacctcatcagcacggtgactggaatttttctgataagggtactgaagggggtcggcgagttggacctacacgccccgactctgccatcccacagaccccttttctacatccttgtgtacctcatctccactgccgtggtaacgagatggaacttggacgtagccggaatactgctacagtgcgccccaacacttctaatggtattcacgatgtgggctgacatcctcaccctaattctcatactgcccacctatgagttgacaaagttatattaccttaaggaagtgaagatagggactgaaagaggatggctgtggaaaactaattacaagagggtaaatgacatctatgaagtcgaccaagctggcgaaggggtttacctcttcccttcgaaacaaaagactggagctataacgagcaccgtgttgccgttgatcaaagccatactcattagctgcatcagtaacaagtggcaattcatatatttactgtacttgatatttgaggtgacctactaccttcacaagaaaatcatagatgagatagccggcgggaccaacttcgtttcgagactagtggcggctttaattgaagttaattgggctctagacaatgaagaagtcaaaggcctaaagaagtttttcttgttgtctagtagggtcaaagagttggttatcaaacacaaagtgaggaatgaagtaatggtccgctggtttgaagatgaagagatctacgggatgccaaagctgatcggcctggttaaggcagcaacactgagcaaaaacaaacactgtattttgtgcaccgtctgtgaggacagagattggcggggagaaacttgcccgaaatgcgggcgttttggaccaccagtgatctgtggtatgaccctagctgacttcgaggaaaaacactataagaggattttcatcagggaggaccaatcagacgggccgctcagggaggagcatgcagggtacttgcagtacaaagctaggggtcaattgtttctgaggaatctcccagtgttggcaacaaaagtcaagatgcttctggttggcaacctagggacagaggttggggacctggagcaccttggctgggtgctcagagggcccgctgtctgcaagaaggttactgaacatgaaaagtgtgctacgtctataatggataagttgactgccttctttggtgtcatgccaaggggtaccacccccagagctcctgtaagatttcccacctcccttctaaagataagaagagggctggagacgggttgggcttatacacaccaaggcggtatcagctcagttgaccatgttacttgcgggaaagacctgctagtgtgtgacaccatgggtcggacaagggttgtctgccaatcgaataacaagatgactgacgagtctgaatacggagtcaaaaccgactcagggtgcccagagggagccaggtgctacgtgttcaaccctgaggcagttaatatatcaggcactaaaggagccatggtccacttacagaaaactggtggagaattcacctgtgtaacagcatcagggaccccagctttcttcgatctcaagaacctcaagggttggtcagggctaccgatatttgaagcatcgagtggaagggtagtcggaagggtcaaggtcgggaagaacgaagactctaaaccaaccaaactcatgagtgggatacaaacggtctctaaaagtgccactgatttgacggagatggtgaagaagataacaaccatgaacaggggggagttcagacaaataaccttggccacaggtgctggaaaaactacagagctacccaggtcagtcatagaagagatagggagacacaaaagggtgctggtattgatcccccttagggccgcagcagaatcagtataccaatacatgaggcagaaacacccgagtatagcattcaacctgaggataggggagatggaggaaggatacatggccacgggaataacctatgcttcttatggttacttttgccagatgccacaacccaagttgagagccgcaatggtagagtattcctacatctttctagacgagtaccattgcgccaccccggaacaattggctatcatggggaagatccacagattctcagagaacctgcgggtggtcgctatgacagcgacgccggcaggcacggtaacaactactgggcagaaacaccctatagaggagttcatagccccggaagtgatgaaaggagaagacttaggttcagagtacttagacatcgccggactaaagataccagtagaggagatgaagaacaacatgctagtttttgtaccaactaggaacatggcagtggaggcggcaaagaaattgaaagccaaaggatataactcaggctattactacagtggtgaggacccatccaacctgagggtagttacatcgcagtccccatacgtggtggtagcgaccaacgcaatagaatcaggcgtcactctcccagaccttgatgtggtcgttgacacaggactcaagtgtgagaaaagaatccgactgtcacccaagatgcctttcatagtgactggcctgaaaagaatggccgtcactattggggaacaagcccagagaagagggagggttggaagagtaaagcccgggagatactacaggagccaagaaactccagtcggctctaaggactaccactatgacttgctgcaagcccaaaggtacggtatagaagatgggataaatatcaccaaatccttcagagagatgaactacgattggagcctttatgaggaagatagcctgatgataacacaactggaaatcctcaacaatctgttgatatcagaggagctgccggtagcagtaaaaaatataatggctaggactgaccacccagaaccaattcaactagcgtataatagctacgagacacaggtaccagtgttgttcccaaagataaggaatggagaggtgactgacacttacgacaactacaccttcctcaatgcaagaaaattgggagatgatgtacccccctatgtgtatgccacagaagatgaggacttggcagtggaactgttaggcctagattggccagaccctggaaaccaaggcactgtggaggctggcagagcactaaaacaggtggtcggcctatcaacagccgagaatgccctgctagtggccttgtttggctacgtagggtaccaggccctctcaaaaaggcatatacctgtagtcacagacatatattcagttgaagatcacaggttggaagatacaacacacctacagtatgccccgaacgccatcaagacggaggggaaggagactgaattgaaggagttggctcaaggggatgtgcagagatgtgtggaagcagtggccaattatgcgagtgagggcatccaatttattaagtcacaggcactgaaggtgagagagaccccgacttacaaagagacaatgaatacagtggcagactatgtgaaaaagttcattgaggcactgtcagatagcaaggaagacatcttgaaatatgggctgtggggtgtacacacggccttgtataagagcattggtgccagactaggtcacgaaactgcgttcgcaactttagtcgtgaaatggttggcatttgggggggaatcagtaacagatcacataaagcaagcagccacagacttggttgtttactatattatcaacagacctcaattcccaggggacacggagacacaacaagaagggaggaaatttgtggccagcttactggtctcagccctagcaacctacacatacaaaagctggaactacaacaatctgtccaagatagttgaacctgctttggccactttgccctacgccgccaaagcccttaaattattcgcccctacccgactggagagcgtcgtcatattgagcaccgcaatctacaaaacatatctatcaatcaggcgaggcaaaagtgatggattgcttggtacaggggttagtgcggctatggagattatgtcacaaaatccagtatcagtgggcatagcagtcatgctgggggttggggctgtagcagcccataatgcaattgaagccagtgagcagaagagaacactactaatgaaagtctttgtgaaaaacttcttggaccaagcagccacggatgaactagtcaaagagagccctgagaaaataataatggccctgtttgaagcagtgcagacggtaggcaaccctctcagactggtgtaccacctatatggagttttttataaagggtgggaagcaaaagagttggcccaaaggacagccggcaggaacctcttcaccttaataatgttcgaggctgtggaactactgggagtagatagtgaagggaaaatccgccagctctcaagtaactacatattagagctcctgtacaagttccgtgacgacatcaaatctagtgtgagggagatagcaatcagttgggcccctgcccccttcagctgcgactggacaccaacagatgacagaataggacttccccatgacaattatctccagatggagacaagatgtccttgtggttacaggatgaaagcagtaaaaacctgtgccggggagttgagactcctggaagaggggggttcattcctctgcaggaataaattcggcagagggtcacggaattatagggtgacaaaatattatgatgacaacttatcagaaataaaaccagtgataagaatggaaggacacgtggaactgtatcataaaggggccactttcaaactggactttgacaacagtaaaacagtactggcaacagatagatgggaagtcgaccattccaccctgatcagggtgctcaagaggcacacaggggctggatttcaaggggcgtatctgggtgagaaacccaatcacaagcacctgatagagagagactgtgcaacgatcacaaaagacaaggtctgcttcatcaaaatgaagagggggtgcgcgttcacctatgacttatcactccacaaccttacccggctaatcgaattggtacataagaacaacctggatgacagagaaatccctgccgttacggtcacaacctggctggcctacacattcgtgaatgaagacatagggaccataaaaccagtcttcggggaaaaagtaacaccggagaagcaggaggaggtagccttgcagcctgcagtggtggtggacacaacagatgtggccgtgaccgtggtgggggaaacctccactatgaccacaggggagacccccacaatatttaccagcttaggttcggactctaagtttcaacaagtcctgaaactgggggtggatgagggtcaatatcccgggcccaggcagcagagagcaagtttgctcgaagctgtacaaggtgtagatgagaggccctcggtactaatattggggtctgataaggccacctccaatagggtgaagaccgcaaagaatgtgaagatattcaggagcagagaccccctggaactgagagagatgatgagaagagggaagatcctaatcatagccctgtgtaaggtggacaccgctctgctgaaatttgttgattacaaaggtaccttcctgaccagagagaccctagaggcattaagtctgggcaaacctaagaaaagagacataactaagacagaggcacgatggttgttgtgcctcgaaggtcaattagaagagctgcctgactggtttgcagccaaggagcccatattcttagaagccaacatcaaacgtgacaagtatcatctggtgggagacatagctacaatcaaagaaaaagccaagcaactgggggcaacagactccacaaagatatcaaaagaggttggcgcgaaagtgtattctatgaagttgagtaactgggtgatacaggaagagaataaacagggcagcttaacccccttgtttgaagagctcctgcaacagtgcccgcccgggggccagaataaaaccacacatatggcctcagcctaccaattggctcaggggaactggatgccagttggttgccacgtgttcatggggaccatacccgctagaagaaccaagactcatccctacgaggcatatgtcaagttgagggagttggtggaagaacataagatgaagacatcatgtggcggttcaggcctatgtaagcacaacgaatgggtaattcgtaagatcaagcatcaagggaacctgaggaccagacacatgctgaaccccggaaagattgctgagcaactggatagagaagggcacagacacaatgtgtacaacaagaccataggctcagtaatgacagcgactggcatcaggctggaaaagttacccgtggttagagcccagacagatacaactaatttccaccaagcaataagggataaaatagacaaggaagagaacctacagaccccaggcttacacaagaagttaatggaggtattcaatgcattaaaaagacctgagcttgaggcctcttacgacgccgtggagtgggaggaattggagagaggaataaataggaagggtgctgcagggttcttcgaacgcaagaatataggagaggtcctagattcagaaaaatataaggttgaagagatcatagacagtttgaagaaaggtaggagtattaaatattatgaaactgcaatcccaaagaacgaaaagagggatgtcaatgatgactggactgccggagactttgtggatgagaaaaaacccagggtgatacaatatcctgaggccaaaaccaggttagccatcactaaagtgatgtataagtgggtgaaacagaaaccagtagtcatacccggctatgaaggtaagacacctctgttccaaatttttgacaaagtgaaaaaagaatggaaccaattccaaaatccagtggcagtgagctttgacactaaagcatgggatacccaggtgactacaagagacctggaactaataagggatatacagaagttctatttcaagaagaagtggcacaaattcatcgacaccctaaccatgcacatgtcagaagtacccgtgattagtgccgacggggaggtgtatataaggaaagggcagagaggcagtgggcaacctgatacgagcgcaggcaacagtatgctgaatgtgttaacaatggtttatgccttctgtgaggccacgggggtaccctataaaagtttcgacagagtggcaaagatccacgtctgtggggatgatggcttcttgattactgaaagggctcttggtgaaaaatttgcaagtagaggagtccagatcctatatgaagccgggaagccccaaaaaatcactgaaggggacaatatgaaagtggcctatcagtttgacgacatcgagttctgctcccatacaccagtgcaggtaaggtggtcagacaacacctccagttacatgccggggaggaacacaaccacaatactggcaaaaatggctacaaggttggattccagtggtgagagaggcactatagcatatgagaaggcagtggcattcagtttcttgttgatgtactcctggaacccactgattagaaggatatgcttattggtgttatcaactgaaatgcaagtgagaccagggaagtcaaccacctattactatgaaggagacccaatatctgcttacaaggaggtcatcggccataatctctttgaccttaaaagaacaagtttcgagaagctagctaagctaaatcttagtatgtccacactcggggtatggaccaggcataccagcaaaagattactacaagactgtgtcaatgtcggcaccaaagagggcaactggctggttaacgcagacagactggtgagtagtaagacagggaataggtatatacctggagagggtcatacccaacaaggaaaacattatgaagagctggtattggcaagaaaaccgaccagtaattttgaagggactgatagatataatttgggcccaatagtcaacgtagtgttgaggaggctgagagtcatgatgatggctcttataggaaggggggtgtgagagcggccggctcttgaccgggccctatcagtataaccctgttgtaaataacgctaacttattatttatctagacactactatttatttatttatttatttattgaatgagcaaggaatggtacgaactacctcatgttaccacactacactcattttaacagcactttagctgaggaaaaaantcctgacgtccatagttggactaaggtaatttcctaacggccc

SEQ ID NO: 6 - DNA, 11804 bp, Artificial Sequence
  GD18-ddNproErnsH-KARD
  misc_feature (11761)..(11761): n is a, c, g, or t
gtatacgaggttagttcgttctcgtgtacaatattggacaagaccaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggtgctgcagaggcccactaacaggctagtataaaaaatctctgctgtacatggcacatggagttgaattctgatgatggcgcaggtggcagcaaggataagaaaccagacaggatgaacaagggtaaattaaaaatagccccaaaagagcatgagaaggacagcaaaaccaagccacctgatgccacgatcgtggtagagggagtaaaataccaagtgaaaaagaaaggcaaagtcaaagggaagaacactcaagatggcctataccataataagaacaaaccaccagagtccaggaagaaactagaaaaagccctactggcttgggcagtgataacaatcgtactgtaccagcctgtagcggccgagaatataactcagtggaacctgagtgacaatggcacaagcggtatccagcaagccatgtatcttagaggggttaataggagtctacatgggatctggcctgaaaaaatatgcaaaggggtccctactcacctggctactgatacggagctgacagagatacgagggatgatggacgccagcgagaggacaaactatacgtgttgcaggttgcagagacacgaatggaataaaggatggtgtaactggtacaacatagacccctggattcaattaatgaacaggacccaagcaaatctgacagaaggccccccggataaggagtgcgccgtgacttgcaggtatgacaaaaatgctgacgttaacgtggtcacccaggccaggaatagaccaaccactctgaccggctgcaagaaaggaaaaaacttttcatttgcaggtacgattatagagggcccatgcaatttcaacgtctccgtggaggacatcctatatggggaccatgagtgtggcagcctgttccaggacacagctctgtacctattagatggaatgaccaacactatagagaaagccaggcagggtgcggcaagagttacatcctggcttgggaggcaactcagaaccacagggaagaagttggagagaggaagcaaaacctggttcggcgcctacgccctgtcaccttactgtaacgtaacaaggaaagtggggtacatatggtatacgaacaattgcactccggcatgtctcccaaaaaacacgaaaataataggtccaggaaaattcgataccaacgcggaagatgggaagatacttcatgaaatggggggccacctatcagaatttttgttgctttctttagtaatcctgtctgacttcgccccggagacggccagtacgctatacctaatcttacactatgcaatccctcagtcccatgaagagcctgaaggttgcgatacgaaccagctcaatctaacagtgggacttaggacagaagatgtggtaccatcatcagtttggaatatcggcaaatatgtgtgtgtcagaccagactggtggccgtatgaaactaaggtggctttgctgtttgaggaggcaggacaggttataaaactagctctacgggcactgagagatttaactagagtctggaacagtgcatcaactactgcgttcctcatctgcttgataaaaatattaagaggacaggttgtgcaaggtataatatggctgctgctggtaactggggcacaagggcggttgacctgtaaggaagactacaggtatgcgatatcaaagaccaatgagatagggccgcttgccgctagggacctcactaccacctggaaagaatacaaccatggtttacagctggatgacgggactgtcagggccacttgcactgcagggtcctttaaagttatagcacttaatgtggttagtaggaggtacctggcatcattacacaagagggctttgcccacctcagtaacatttgaactcctatttgatgggaccagcccagtaattgaggagatgggagacgactttggatttgggctgtgcccttttgacacgatccctgtggtcaaagggaagtataacaccaccttattaaacggcagtgctttctatctagtctgccctataggatggacgggtgttatagagtgcacggcagtaagccccacaaccttgagaacagaagtggtgaagaccttcaagagagagaagcctttccctcacagagtggactgcgtgaccactatagtagaaaaagaagacctgttctattgcaggctggggggtaattggacatgcgtgaaaggtgacccggtgacctacacggggggccaagtaaaacaatgcaggtggtgcggtttcaacttcaaggagcctgatgggctcccacactaccccataggcaagtgcatcctagcaaatgagacgggatacagggtagtggactccacagactgcaacagagacggcgtcgtcatcggcactgaaggagagcatgagtgcttgatcggcaacaccaccgtcaaggtgcacgcgctggacggaagactggcccctatgccttgcagacccaaagaaatcgtatctagtgcgggacctgtaaggaaaacttcctgcacatttaactatacaaagactttgaggaacaagtactatgagcccagggacagctacttccagcaatacatgcttaagggcgagtatcaatactggtttgatctggacgtgacagaccaccacacagactacttcgccgagtttgttgtcttggtggtagtggcactattagggggaaggtacgtcctgtggctaatagtgacctatatagttctaacagagcaactcgctgctggtttacagctaggccagggcgaggtggtgctgatagggaacttaatcacccacatggacaacgaggtagtggtatacttcttactgctttacctaataataagagacgagcccataaagaaatggatactactgctgttccatgccatgaccaacaacccagttaagaccatgacagtagcattgctaatgataagcggggtagccaagggtgggaaaatagatggtggttggcagaggcagccggagaccaattttgacatccaattcgcactggcagtcataatagtcgtcgtgatgttgttggcaaagagagatccgactacttttcccctggtgatcactgtggcaaccttgagaacggctaagataaccagtggtttcagcacagatctagccatagccacagtgtcggcaactttgctaacctggacttatatcagtgactactacaaatacaagacttggctacagtacctcatcagcacggtgactggaatttttctgataagggtactgaagggggtcggcgagttggacctacacgccccgactctgccatcccacagaccccttttctacatccttgtgtacctcatctccactgccgtggtaacgagatggaacttggacgtagccggaatactgctacagtgcgccccaacacttctaatggtattcacgatgtgggctgacatcctcaccctaattctcatactgcccacctatgagttgacaaagttatattaccttaaggaagtgaagatagggactgaaagaggatggctgtggaaaactaattacaagagggtaaatgacatctatgaagtcgaccaagctggcgaaggggtttacctcttcccttcgaaacaaaagactggagctataacgagcaccgtgttgccgttgatcaaagccatactcattagctgcatcagtaacaagtggcaattcatatatttactgtacttgatatttgaggtgtcctactaccttcacaagaaaatcatagatgagatagccggcgggaccaacttcgtttcgagactagtggcggctttaattgaagttaattgggctctagacaatgaagaagtcaaaggcctaaagaagtttttcttgttgtctagtagggtcaaagagttggttatcaaacacaaagtgaggaatgaagtaatggtccgctggtttgaagatgaagagatctacgggatgccaaagctgatcggcctggttaaggcagcaacactgagcaaaaacaaacactgtattttgtgcaccgtctgtgaggacagagattggcggggagaaacttgcccgaaatgcgggcgttttggaccaccagtgatctgtggtatgaccctagctgacttcgaggaaaaacactataagaggattttcatcagggaggaccaatcagacgggccgctcagggaggagcatgcagggtacttgcagtacaaagctaggggtcaattgtttctgaggaatctcccagtgttggcaacaaaagtcaagatgcttctggttggcaacctagggacagaggttggggacctggagcaccttggctgggtgctcagagggcccgctgtctgcaagaaggttactgaacatgaaaagtgtgctacgtctataatggataagttgactgccttctttggtgtcatgccaaggggtaccacccccagagctcctgtaagatttcccacctcccttctaaagataagaagagggctggagacgggttgggcttatacacaccaaggcggtatcagctcagttgaccatgttacttgcgggaaagacctgctagtgtgtgacaccatgggtcggacaagggttgtctgccaatcgaataacaagatgactgacgagtctgaatacggagtcaaaaccgactcagggtgcccagagggagccaggtgctacgtgttcaaccctgaggcagttaatatatcaggcactaaaggagccatggtccacttacagaaaactggtggagaattcacctgtgtaacagcatcagggaccccagctttcttcgatctcaagaacctcaagggttggtcagggctaccgatatttgaagcatcgagtggaagggtagtcggaagggtcaaggtcgggaagaacgaagactctaaaccaaccaaactcatgagtgggatacaaacggtctctaaaagtgccactgatttgacggagatggtgaagaagataacaaccatgaacaggggggagttcagacaaataaccttggccacaggtgctggaaaaactacagagctacccaggtcagtcatagaagagatagggagacacaaaagggtgctggtattgatcccccttagggccgcagcagaatcagtataccaatacatgaggcagaaacacccgagtatagcattcaacctgaggataggggagatggaggaaggatacatggccacgggaataacctatgcttcttatggttacttttgccagatgccacaacccaagttgagagccgcaatggtagagtattcctacatctttctagacgagtaccattgcgccaccccggaacaattggctatcatggggaagatccacagattctcagagaacctgcgggtggtcgctatgacagcgacgccggcaggcacggtaacaactactgggcagaaacaccctatagaggagttcatagccccggaagtgatgaaaggagaagacttaggttcagagtacttagacatcgccggactaaagataccagtagaggagatgaagaacaacatgctagtttttgtaccaactaggaacatggcagtggaggcggcaaagaaattgaaagccaaaggatataactcaggctattactacagtggtgaggacccatccaacctgagggtagttacatcgcagtccccatacgtggtggtagcgaccaacgcaatagaatcaggcgtcactctcccagaccttgatgtggtcgttgacacaggactcaagtgtgagaaaagaatccgactgtcacccaagatgcctttcatagtgactggcctgaaaagaatggccgtcactattggggaacaagcccagagaagagggagggttggaagagtaaagcccgggagatactacaggagccaagaaactccagtcggctctaaggactaccactatgacttgctgcaagcccaaaggtacggtatagaagatgggataaatatcaccaaatccttcagagagatgaactacgattggagcctttatgaggaagatagcctgatgataacacaactggaaatcctcaacaatctgttgatatcagaggagctgccggtagcagtaaaaaatataatggctaggactgaccacccagaaccaattcaactagcgtataatagctacgagacacaggtaccagtgttgttcccaaagataaggaatggagaggtgactgacacttacgacaactacaccttcctcaatgcaagaaaattgggagatgatgtacccccctatgtgtatgccacagaagatgaggacttggcagtggaactgttaggcctagattggccagaccctggaaaccaaggcactgtggaggctggcagagcactaaaacaggtggtcggcctatcaacagccgagaatgccctgctagtggccttgtttggctacgtagggtaccaggccctctcaaaaaggcatatacctgtagtcacagacatatattcagttgaagatcacaggttggaagatacaacacacctacagtatgccccgaacgccatcaagacggaggggaaggagactgaattgaaggagttggctcaaggggatgtgcagagatgtgtggaagcagtggccaattatgcgagtgagggcatccaatttattaagtcacaggcactgaaggtgagagagaccccgacttacaaagagacaatgaatacagtggcagactatgtgaaaaagttcattgaggcactgtcagatagcaaggaagacatcttgaaatatgggctgtggggtgtacacacggccttgtataagagcattggtgccagactaggtcacgaaactgcgttcgcaactttagtcgtgaaatggttggcatttgggggggaatcagtaacagatcacataaagcaagcagccacagacttggttgtttactatattatcaacagacctcaattcccaggggacacggagacacaacaagaagggaggaaatttgtggccagcttactggtctcagccctagcaacctacacatacaaaagctggaactacaacaatctgtccaagatagttgaacctgctttggccactttgccctacgccgccaaagcccttaaattattcgcccctacccgactggagagcgtcgtcatattgagcaccgcaatctacaaaacatatctatcaatcaggcgaggcaaaagtgatggattgcttggtacaggggttagtgcggctatggagattatgtcacaaaatccagtatcagtgggcatagcagtcatgctgggggttggggctgtagcagcccataatgcaattgaagccagtgagcagaagagaacactactaatgaaagtctttgtgaaaaacttcttggaccaagcagccacggatgaactagtcaaagagagccctgagaaaataataatggccctgtttgaagcagtgcagacggtaggcaaccctctcagactggtgtaccacctatatggagttttttataaagggtgggaagcaaaagagttggcccaaaggacagccggcaggaacctcttcaccttaataatgttcgaggctgtggaactactgggagtagatagtgaagggaaaatccgccagctctcaagtaactacatattagagctcctgtacaagttccgtgacgacatcaaatctagtgtgagggagatagcaatcagttgggcccctgcccccttcagctgcgactggacaccaacagatgacagaataggacttccccatgacaattatctccagatggagacaagatgtccttgtggttacaggatgaaagcagtaaaaacctgtgccggggagttgagactcctggaagaggggggttcattcctctgcaggaataaattcggcagagggtcacggaattatagggtgacaaaatattatgatgacaacttatcagaaataaaaccagtgataagaatggaaggacacgtggaactgtatcataaaggggccactttcaaactggactttgacaacagtaaaacagtactggcaacagatagatgggaagtcgaccattccaccctgatcagggtgctcaagaggcacacaggggctggatttcaaggggcgtatctgggtgagaaacccaatcacaagcacctgatagagagagactgtgcaacgatcacaaaagacaaggtctgcttcatcaaaatgaagagggggtgcgcgttcacctatgacttatcactccacaaccttacccggctaatcgaattggtacataagaacaacctggatgacagagaaatccctgccgttacggtcacaacctggctggcctacacattcgtgaatgaagacatagggaccataaaaccagtcttcggggaaaaagtaacaccggagaagcaggaggaggtagccttgcagcctgcagtggtggtggacacaacagatgtggccgtgaccgtggtgggggaaacctccactatgaccacaggggagacccccacaatatttaccagcttaggttcggactctaagtttcaacaagtcctgaaactgggggtggatgagggtcaatatcccgggcccaggcagcagagagcaagtttgctcgaagctgtacaaggtgtagatgagaggccctcggtactaatattggggtctgataaggccacctccaatagggtgaagaccgcaaagaatgtgaagatattcaggagcagagaccccctggaactgagagagatgatgagaagagggaagatcctaatcatagccctgtgtaaggtggacaccgctctgctgaaatttgttgattacaaaggtaccttcctgaccagagagaccctagaggcattaagtctgggcaaacctaagaaaagagacataactaagacagaggcacgatggttgttgtgcctcgaaggtcaattagaagagctgcctgactggtttgcagccaaggagcccatattcttagaagccaacatcaaacgtgacaagtatcatctggtgggagacatagctacaatcaaagaaaaagccaagcaactgggggcaacagactccacaaagatatcaaaagaggttggcgcgaaagtgtattctatgaagttgagtaactgggtgatacaggaagagaataaacagggcagcttaacccccttgtttgaagagctcctgcaacagtgcccgcccgggggccagaataaaaccacacatatggcctcagcctaccaattggctcaggggaactggatgccagttggttgccacgtgttcatggggaccatacccgctagaagaaccaagactcatccctacgaggcatatgtcaagttgagggagttggtggaagaacataagatgaagacatcatgtggcggttcaggcctatgtaagcacaacgaatgggtaattcgtaagatcaagcatcaagggaacctgaggaccagacacatgctgaaccccggaaagattgctgagcaactggatagagaagggcacagacacaatgtgtacaacaagaccataggctcagtaatgacagcgactggcatcaggctggaaaagttacccgtggttagagcccagacagatacaactaatttccaccaagcaataagggataaaatagacaaggaagagaacctacagaccccaggcttacacaagaagttaatggaggtattcaatgcattaaaaagacctgagcttgaggcctcttacgacgccgtggagtgggaggaattggagagaggaataaataggaagggtgctgcagggttcttcgaacgcaagaatataggagaggtcctagattcagaaaaatataaggttgaagagatcatagacagtttgaagaaaggtaggagtattaaatattatgaaactgcaatcccaaagaacgaaaagagggatgtcaatgatgactggactgccggagactttgtggatgagaaaaaacccagggtgatacaatatcctgaggccaaaaccaggttagccatcactaaagtgatgtataagtgggtgaaacagaaaccagtagtcatacccggctatgaaggtaagacacctctgttccaaatttttgacaaagtgaaaaaagaatggaaccaattccaaaatccagtggcagtgagctttgacactaaagcatgggatacccaggtgactacaagagacctggaactaataagggatatacagaagttctatttcaagaagaagtggcacaaattcatcgacaccctaaccatgcacatgtcagaagtacccgtgattagtgccgacggggaggtgtatataaggaaagggcagagaggcagtgggcaacctgatacgagcgcaggcaacagtatgctgaatgtgttaacaatggtttatgccttctgtgaggccacgggggtaccctataaaagtttcgacagagtggcaaagatccacgtctgtggggatgatggcttcttgattactgaaagggctcttggtgaaaaatttgcaagtagaggagtccagatcctatatgaagccgggaagccccaaaaaatcactgaaggggacaatatgaaagtggcctatcagtttgacgacatcgagttctgctcccatacaccagtgcaggtaaggtggtcagacaacacctccagttacatgccggggaggaacacaaccacaatactggcaaaaatggctacaaggttggattccagtggtgagagaggcactatagcatatgagaaggcagtggcattcagtttcttgttgatgtactcctggaacccactgattagaaggatatgcttattggtgttatcaactgaaatgcaagtgagaccagggaagtcaaccacctattactatgaaggagacccaatatctgcttacaaggaggtcatcggccataatctctttgaccttaaaagaacaagtttcgagaagctagctaagctaaatcttagtatgtccacactcggggtatggaccaggcataccagcaaaagattactacaagactgtgtcaatgtcggcaccaaagagggcaactggctggttaacgcagacagactggtgagtagtaagacagggaataggtatatacctggagagggtcatacccaacaaggaaaacattatgaagagctggtattggcaagaaaaccgaccagtaattttgaagggactgatagatataatttgggcccaatagtcaacgtagtgttgaggaggctgagagtcatgatgatggctcttataggaaggggggtgtgagagcggccggctcttgaccgggccctatcagtataaccctgttgtaaataacgctaacttattatttatctagacactactatttatttatttatttatttattgaatgagcaaggaatggtacgaactacctcatgttaccacactacactcattttaacagcactttagctgaggaaaaaantcctgacgtccatagttggactaaggtaatttcctaacggccc

SEQ ID NO: 7 - protein, 373 aa, Artificial Sequence
  QZ07-E2
RLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 8 - protein, 373 aa, Artificial Sequence
  GD18-E2
RLTCKEDYRYAISSTNEIGPLGAEGLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 9 - protein, 135 aa, Artificial Sequence
  variable region of heavy chain of mAb 6B8
MDSRLNLVFLVLILKGVQCDVQLVESGGGLVQPGGSRKLSCAASGFTFSSFGMHWVRQAPEKGLEWVAYISSDSFTIYYADTMKGRFTISRDNPQNTLFLQMTSLRSEDTAMYYCARGDLPFAYWGQGTLVTVSA

SEQ ID NO: 10 - protein, 131 aa, Artificial Sequence
  variable region of light chain of mAb 6B8
MGIKMETHSQVFVYMLLWLSGVEGDIVMTQSHKFMSTSVGDRVSITCKASQAVGTAVAWYQQKPGQSPKLLIYWASTRHTGVPDRFTGSGSGTDFTLTISNVQSEDLADYFCHQYSSYPLTFGAGTKLELK

SEQ ID NO: 11 - protein, 12 aa, Artificial Sequence
  6B8 epitope for GD18 or QZ07 or GD191
STNEIGPLGAEG

SEQ ID NO: 12 - protein, 12 aa, Artificial Sequence
  6B8 epitope for C strain
STDEIGLLGAGG

SEQ ID NO: 13 - protein, 12 aa, Artificial Sequence
  mutated 6B8 epitope with KRD for GD18 or QZ07 or GD191
KTNEIGPLGARD

SEQ ID NO: 14 - protein, 12 aa, Artificial Sequence
  mutated 6B8 epitope with KARD for GD18 or QZ07 or GD191
KTNEIGPLAARD

SEQ ID NO: 15 - protein, 168 aa, Artificial Sequence
  Npro of GD18
MELNHFELLYKTNKQKPIGVEEPVYDIAGRPFFGDPSEVHPQSTLKLPHDRGRGNIRTTLKNLPRKGDCRSGNHLGPVSGIYVKPGPVFYQDYMGPVYHRAPLEFFEEAQLCEVTKRIGRVTGSDGKLYHIYVCIDGCILLKLAKRGTPRSLKWTRNFTDCPLWVTSC

SEQ ID NO: 16 - protein, 227 aa, Artificial Sequence
  Erns of GD18
ENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNADVNVVTQARNRPTTLTGCKKGKNFSFAGTIIEGPCNFNVSVEDILYGDHECGSLFQDTALYLLDGMTNTIEKARQGAARVTSWLGRQLSTTGKKLERGSKTWFGAYA

SEQ ID NO: 17 - protein, 3898 aa, Artificial Sequence
  polyprotein of GD18
MELNHFELLYKTNKQKPIGVEEPVYDIAGRPFFGDPSEVHPQSTLKLPHDRGRGNIRTTLKNLPRKGDCRSGNHLGPVSGIYVKPGPVFYQDYMGPVYHRAPLEFFEEAQLCEVTKRIGRVTGSDGKLYHIYVCIDGCILLKLAKRGTPRSLKWTRNFTDCPLWVTSCSDDGAGGSKDKKPDRMNKGKLKIAPKEHEKDSKTKPPDATIVVEGVKYQVKKKGKVKGKNTQDGLYHNKNKPPESRKKLEKALLAWAVITIVLYQPVAAENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNADVNVVTQARNRPTTLTGCKKGKNFSFAGTIIEGPCNFNVSVEDILYGDHECGSLFQDTALYLLDGMTNTIEKARQGAARVTSWLGRQLSTTGKKLERGSKTWFGAYALSPYCNVTRKVGYIWYTNNCTPACLPKNTKIIGPGKFDTNAEDGKILHEMGGHLSEFLLLSLVILSDFAPETASTLYLILHYAIPQSHEEPEGCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLTCKEDYRYAISSTNEIGPLGAEGLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAGLQLGQGEVVLIGNLITHMDNEVVVYFLLLYLIIRDEPIKKWILLLFHAMTNNPVKTMTVALLMISGVAKGGKIDGGWQRQPETNFDIQFALAVIIVVVMLLAKRDPTTFPLVITVATLRTAKITSGFSTDLAIATVSATLLTWTYISDYYKYKTWLQYLISTVTGIFLIRVLKGVGELDLHAPTLPSHRPLFYILVYLISTAVVTRWNLDVAGILLQCAPTLLMVFTMWADILTLILILPTYELTKLYYLKEVKIGTERGWLWKTNYKRVNDIYEVDQAGEGVYLFPSKQKTGAITSTVLPLIKAILISCISNKWQFIYLLYLIFEVSYYLHKKIIDEIAGGTNFVSRLVAALIEVNWALDNEEVKGLKKFFLLSSRVKELVIKHKVRNEVMVRWFEDEEIYGMPKLIGLVKAATLSKNKHCILCTVCEDRDWRGETCPKCGRFGPPVICGMTLADFEEKHYKRIFIREDQSDGPLREEHAGYLQYKARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMEEGYMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDNYTFLNARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAVANYASEGIQFIKSQALKVRETPTYKETMNTVADYVKKFIEALSDSKEDILKYGLWGVHTALYKSIGARLGHETAFATLVVKWLAFGGESVTDHIKQAATDLVVYYIINRPQFPGDTETQQEGRKFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDDIKSSVREIAISWAPAPFSCDWTPTDDRIGLPHDNYLQMETRCPCGYRMKAVKTCAGELRLLEEGGSFLCRNKFGRGSRNYRVTKYYDDNLSEIKPVIRMEGHVELYHKGATFKLDFDNSKTVLATDRWEVDHSTLIRVLKRHTGAGFQGAYLGEKPNHKHLIERDCATITKDKVCFIKMKRGCAFTYDLSLHNLTRLIELVHKNNLDDREIPAVTVTTWLAYTFVNEDIGTIKPVFGEKVTPEKQEEVALQPAVVVDTTDVAVTVVGETSTMTTGETPTIFTSLGSDSKFQQVLKLGVDEGQYPGPRQQRASLLEAVQGVDERPSVLILGSDKATSNRVKTAKNVKIFRSRDPLELREMMRRGKILIIALCKVDTALLKFVDYKGTFLTRETLEALSLGKPKKRDITKTEARWLLCLEGQLEELPDWFAAKEPIFLEANIKRDKYHLVGDIATIKEKAKQLGATDSTKISKEVGAKVYSMKLSNWVIQEENKQGSLTPLFEELLQQCPPGGQNKTTHMASAYQLAQGNWMPVGCHVFMGTIPARRTKTHPYEAYVKLRELVEEHKMKTSCGGSGLCKHNEWVIRKIKHQGNLRTRHMLNPGKIAEQLDREGHRHNVYNKTIGSVMTATGIRLEKLPVVRAQTDTTNFHQAIRDKIDKEENLQTPGLHKKLMEVFNALKRPELEASYDAVEWEELERGINRKGAAGFFERKNIGEVLDSEKYKVEEIIDSLKKGRSIKYYETAIPKNEKRDVNDDWTAGDFVDEKKPRVIQYPEAKTRLAITKVMYKWVKQKPVVIPGYEGKTPLFQIFDKVKKEWNQFQNPVAVSFDTKAWDTQVTTRDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFASRGVQILYEAGKPQKITEGDNMKVAYQFDDIEFCSHTPVQVRWSDNTSSYMPGRNTTTILAKMATRLDSSGERGTIAYEKAVAFSFLLMYSWNPLIRRICLLVLSTEMQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELVLARKPTSNFEGTDRYNLGPIVNVVLRRLRVMMMALIGRGV

SEQ ID NO: 18 - protein, 168 aa, Artificial Sequence
  Npro of QZ07
MELNHFELLYKTNKQKPMGVEEPVYDIAGRPLFGDPSEVHPQSTLKLPHDRGRGNIRTTLKDLPRKGDCRSGNHLGPVSGIYVKPGPVFYQDYMGPVYHRAPLEFFGEAQFCEVTKRIGRVTGSDGKLYHIYVCIDGCILLKLAKRGAPRSLKWTRNFTDCPLWVTSC

SEQ ID NO: 19 - protein, 227 aa, Artificial Sequence
  Erns of QZ07
ENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNTDVNVVTQARNRPTTLTGCKKGKNFSFAGTVIEGPCNFNVSVEDILYGDHECGSLFQDTALYLLDGMTNTIEKARQGAARVTSWLGRQLRTTGKKLERGSKTWFGAYA

SEQ ID NO: 20 - protein, 3898 aa, Artificial Sequence
  polyprotein of QZ07
MELNHFELLYKTNKQKPMGVEEPVYDIAGRPLFGDPSEVHPQSTLKLPHDRGRGNIRTTLKDLPRKGDCRSGNHLGPVSGIYVKPGPVFYQDYMGPVYHRAPLEFFGEAQFCEVTKRIGRVTGSDGKLYHIYVCIDGCILLKLAKRGAPRSLKWTRNFTDCPLWVTSCSDDGTGDSKDKKPDRMNKGKLKIAPKEHEKDSKTKPPDATIVVEGVKYQVKKKGKVKGKNTQDGLYHNKNKPPESRKKLEKALLAWAVIAIVLYQPVAAENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNTDVNVVTQARNRPTTLTGCKKGKNFSFAGTVIEGPCNFNVSVEDILYGDHECGSLFQDTALYLLDGMTNTIEKARQGAARVTSWLGRQLRTTGKKLERGSKTWFGAYALSPYCNVTRKIGYIWYTNNCTPACLPKNTKIIGPGKFDTNAEDGKILHEMGGHLSEFLLLSLVVLSDFAPETASTLYLILHYAIPQSRDEPEVCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAGLQLGQGEVVLIGNLITHTDNEVVVYFLLLYLIIRDEPIKKWILLLFHAMTNNPVKTMTVALLMISGVAKGGKTDGGWQRQPETNFDIQLALAVIVVVVMLLAKRDPTTFPLVITVATLRTAKITNGFSTDLAIATVSAALLTWTYISDYYKYKTWLQYLISTVTGIFLIRVLKGIGELDMHAPTLPSHRPLFYILVYLISTAVVTRWNLDVAGLLLQCVPTLLMVFTMWADILTLILVLPTYELAKLYYLKEVKIGTERGWLWKTNYKRVNDIYEVDQAGEGVYLFPSKQKTSAITSTMLPLIKAILISCISNKWQLIYLLYLIFEVSYYLHKKVIDEIAGGTNFVSRLVAALIEVNWAFDNEEVKGLKKFFLLSSRVKELVIKHKVRNEVVARWFGDEEIYGMPKLIGLVKAATLSKNKHCILCTVCEDRDWRGETCPKCGRFGPPVICGMTLADFEEKHYKRIFIREDQSDGPLREERAGYLQYRARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNDDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMKEGHMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDTYTFINARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAMTNYARGGIQFMKSQALQVRETPTYKETMNTVADYVKKFIEALSDSKEDILKYGLWGVHTALYKSIGARLGHETAFATLAVKWLAFGGESIADHIKQAATDLVVYYIINRPQFPGDTETQQEGRNFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDSIKSSVREIAISWAPAPFSCDWTPTDDRIGLPHNNYLQMETRCPCGYRMKAVKTCAGELRLLEEGGSFLCRNKFGRGSRNYRVTKYYDDNLSEIKPVIRMEGHVELYYKGATIKLDFNNSKTVLATDKWEVDHSTLVRALKRHTGAGYQGAYMGEKPNHKHLIERDCATITKDKVYFIKMKRGCAFTYDLSLHNLTRLIELVHKNDLEDREIPAVTVTTWLAYTFVNEDIGTIKPVFGEKVTPEKQEEVALQPAVVVDTTDVAVTVVGETSTMTTGETPTAFTSLGSDSKVQQVLKLGVDEGQYPGPSQQRASLLDAIQGVDERPSVLILGSDKATSNRVKTAKNVKIFRSRDPLELREMMRRGKILVIALCKVDTALLKFVDYKGTFLTRETLEALSLGKPKKKNITKTEAQWLLCLENQIEELPDWFAAEEPVFLEANIKRDKYHLVGDIATIKEKAKQLGATDSTKISKEVGAKVYSMKLSNWVIQEENKQGSLAPLFEELLQQCPPGGQNKTTHMVSAYQLAQGNWMPVGCHVFMGTIPARRTKTHPYEAYVKLRELVDEYKMKTLCGGSGLSKHNEWVIRKIKHQGNLRTKHMLNPGKVAEQLLREGHRHNVYNKTIGSVMTATGIRLEKLPVVRAQTDTTNFHQAIRDKIDKEENLQTPGLHKKLMEVFNALKRPDLEASYDAVEWEELEKGINRKGAAGFFEHKNIGEVLDSEKNKVEEIIDSLRKGRSIRYYETAIPKNEKRDVNDDWTAGDFVDEKKPRVIQYPEAKTRLAITKVMYKWVKQKPVVIPGYEGKTPLFQIFDKVKKEWDQFQNPVAVSFDTKAWDTQVTTGDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFSSKGVQILYEAGKPQKITEGDKMKVAYQFDDIEFCSHTPVQVRWSDNTSSYMPGRNTTTILAKMATRLDSSGERGTIAYEKAVAFSFLLMYSWNPLIRRICLLVLSTELQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELILARKPISNFEGTDRYNLGPIVNVVLRRLRVMMMALIGRGV

SEQ ID NO: 21 - protein, 3897 aa, Artificial Sequence
  polyprotein of QZ07-sdErnsH-KARD
MELNHFELLYKTNKQKPMGVEEPVYDIAGRPLFGDPSEVHPQSTLKLPHDRGRGNIRTTLKDLPRKGDCRSGNHLGPVSGIYVKPGPVFYQDYMGPVYHRAPLEFFGEAQFCEVTKRIGRVTGSDGKLYHIYVCIDGCILLKLAKRGAPRSLKWTRNFTDCPLWVTSCSDDGTGDSKDKKPDRMNKGKLKIAPKEHEKDSKTKPPDATIVVEGVKYQVKKKGKVKGKNTQDGLYHNKNKPPESRKKLEKALLAWAVIAIVLYQPVAAENITQWNLSDNGTSGIQQAMYLRGVNRSLHGIWPEKICKGVPTHLATDTELTEIRGMMDASERTNYTCCRLQRHEWNKGWCNWYNIDPWIQLMNRTQANLTEGPPDKECAVTCRYDKNTDVNVVTQARNRPTTLTGCKKGKNFSFAGTVIEGPCNFNVSVEDILYGDHECGSLFQDTALYLLDGMTNTIEKARQGAARVTSWLGRQLRTTGKKLERGSKTWFGAYALSPYCNVTRKIGYIWYTNNCTPACLPKNTKIIGPGKFDTNAEDGKILHEMGGHLSEFLLLSLVVLSDFAPETASTLYLILHYAIPQSRDEPEVCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLSCKEDYRYAISKTNEIGPLAARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAGLQLGQGEVVLIGNLITHTDNEVVVYFLLLYLIIRDEPIKKWILLLFHAMTNNPVKTMTVALLMISGVAKGGKTDGGWQRQPETNFDIQLALAVIVVVVMLLAKRDPTTFPLVITVATLRTAKITNGFSTDLAIATVSAALLTWTYISDYYKYKTWLQYLISTVTGIFLIRVLKGIGELDMHAPTLPSHRPLFYILVYLISTAVVTRWNLDVAGLLLQCVPTLLMVFTMWADILTLILVLPTYELAKLYYLKEVKIGTERGWLWKTNYKRVNDIYEVDQAGEGVYLFPSKQKTSAITSTMLPLIKAILISCISNKWQLIYLLYLIFEVSYYLHKKVIDEIAGGTNFVSRLVAALIEVNWAFDNEEVKGLKKFFLLSSRVKELVIKHKVRNEVVARWFGDEEIYGMPKLIGLVKAATLSKNKHCILCTVCEDRDWRGETCPKCGRFGPPVICGMTLADFEEKHYKRIFIREDQSDGPLREERAGYLQYRARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNDDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMKEGHMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDTYTFINARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAMTNYARGGIQFMKSQALQVRETPTYKETMNTVADYVKKFIEALSDSKEDILKYGLWGVHTALYKSIGARLGHETAFATLAVKWLAFGGESIADHIKQAATDLVVYYIINRPQFPGDTETQQEGRNFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDSIKSSVREIAISWAPAPFSCDWTPTDDRIGLPHNNYLQMETRCPCGYRMKAVKTCAGELRLLEEGGSFLCRNKFGRGSRNYRVTKYYDDNLSEIKPVIRMEGHVELYYKGATIKLDFNNSKTVLATDKWEVDHSTLVRALKRHTGAGYQGAYMGEKPNHKHLIERDCATITKDKVYFIKMKRGCAFTYDLSLHNLTRLIELVHKNDLEDREIPAVTVTTWLAYTFVNEDIGTIKPVFGEKVTPEKQEEVALQPAVVVDTTDVAVTVVGETSTMTTGETPTAFTSLGSDSKVQQVLKLGVDEGQYPGPSQQRASLLDAIQGVDERPSVLILGSDKATSNRVKTAKNVKIFRSRDPLELREMMRRGKILVIALCKVDTALLKFVDYKGTFLTRETLEALSLGKPKKKNITKTEAQWLLCLENQIEELPDWFAAEEPVFLEANIKRDKYHLVGDIATIKEKAKQLGATDSTKISKEVGAKVYSMKLSNWVIQEENKQGSLAPLFEELLQQCPPGGQNKTTHMVSAYQLAQGNWMPVGCHVFMGTIPARRTKTHPYEAYVKLRELVDEYKMKTLCGGSGLSKHNEWVIRKIKHQGNLRTKHMLNPGKVAEQLLREGHRHNVYNKTIGSVMTATGIRLEKLPVVRAQTDTTNFHQAIRDKIDKEENLQTPGLHKKLMEVFNALKRPDLEASYDAVEWEELEKGINRKGAAGFFEHKNIGEVLDSEKNKVEEIIDSLRKGRSIRYYETAIPKNEKRDVNDDWTAGDFVDEKKPRVIQYPEAKTRLAITKVMYKWVKQKPVVIPGYEGKTPLFQIFDKVKKEWDQFQNPVAVSFDTKAWDTQVTTGDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFSSKGVQILYEAGKPQKITEGDKMKVAYQFDDIEFCSHTPVQVRWSDNTSSYMPGRNTTTILAKMATRLDSSGERGTIAYEKAVAFSFLLMYSWNPLIRRICLLVLSTELQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELILARKPISNFEGTDRYNLGPIVNVVLRRLRVMMMALIGRGV

SEQ ID NO: 22 - protein, 3896 aa, Artificial Sequence
  polyprotein of QZ07-ddErnsHC-KARD
MELNHFELLYKTNKQKPMGVEEPVYDIAGRPLFGDPSEVHPQSTLKLPHDRGRGNIRTTLKDLPRKGDCRSGNHLGPVSGIYVKPGPVFYQDYMGPVYHRAPLEFFGEAQFCEVTKRIGRVTGSDGKLYHIYVCIDGCILLKLAKRGAPRSLKWTRNFTDCPLWVTSCSDDGTGDSKDKKPDRMNKGKLKIAPKEHEKDSKTKPPDATIVVEGVKYQVKKKGKVKGKNTQDGLYHNKNKPPESRKKLEKALLAWAVIAIVLYQPVAAENITQWNLSDNGTSGIQQAMYLRGVNRSLHGIWPEKICKGVPTHLATDTELTEIRGMMDASERTNYTCCRLQRHEWNKGWCNWYNIDPWIQLMNRTQANLTEGPPDKECAVTCRYDKNTDVNVVTQARNRPTTLTGCKKGKNFSFAGTVIEGPCNFNVSVEDILYGDHEGSLFQDTALYLLDGMTNTIEKARQGAARVTSWLGRQLRTTGKKLERGSKTWFGAYALSPYCNVTRKIGYIWYTNNCTPACLPKNTKIIGPGKFDTNAEDGKILHEMGGHLSEFLLLSLVVLSDFAPETASTLYLILHYAIPQSRDEPEVCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLSCKEDYRYAISKTNEIGPLAARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAGLQLGQGEVVLIGNLITHTDNEVVVYFLLLYLIIRDEPIKKWILLLFHAMTNNPVKTMTVALLMISGVAKGGKTDGGWQRQPETNFDIQLALAVIVVVVMLLAKRDPTTFPLVITVATLRTAKITNGFSTDLAIATVSAALLTWTYISDYYKYKTWLQYLISTVTGIFLIRVLKGIGELDMHAPTLPSHRPLFYILVYLISTAVVTRWNLDVAGLLLQCVPTLLMVFTMWADILTLILVLPTYELAKLYYLKEVKIGTERGWLWKTNYKRVNDIYEVDQAGEGVYLFPSKQKTSAITSTMLPLIKAILISCISNKWQLIYLLYLIFEVSYYLHKKVIDEIAGGTNFVSRLVAALIEVNWAFDNEEVKGLKKFFLLSSRVKELVIKHKVRNEVVARWFGDEEIYGMPKLIGLVKAATLSKNKHCILCTVCEDRDWRGETCPKCGRFGPPVICGMTLADFEEKHYKRIFIREDQSDGPLREERAGYLQYRARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNDDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMKEGHMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDTYTFINARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAMTNYARGGIQFMKSQALQVRETPTYKETMNTVADYVKKFIEALSDSKEDILKYGLWGVHTALYKSIGARLGHETAFATLAVKWLAFGGESIADHIKQAATDLVVYYIINRPQFPGDTETQQEGRNFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDSIKSSVREIAISWAPAPFSCDWTPTDDRIGLPHNNYLQMETRCPCGYRMKAVKTCAGELRLLEEGGSFLCRNKFGRGSRNYRVTKYYDDNLSEIKPVIRMEGHVELYYKGATIKLDFNNSKTVLATDKWEVDHSTLVRALKRHTGAGYQGAYMGEKPNHKHLIERDCATITKDKVYFIKMKRGCAFTYDLSLHNLTRLIELVHKNDLEDREIPAVTVTTWLAYTFVNEDIGTIKPVFGEKVTPEKQEEVALQPAVVVDTTDVAVTVVGETSTMTTGETPTAFTSLGSDSKVQQVLKLGVDEGQYPGPSQQRASLLDAIQGVDERPSVLILGSDKATSNRVKTAKNVKIFRSRDPLELREMMRRGKILVIALCKVDTALLKFVDYKGTFLTRETLEALSLGKPKKKNITKTEAQWLLCLENQIEELPDWFAAEEPVFLEANIKRDKYHLVGDIATIKEKAKQLGATDSTKISKEVGAKVYSMKLSNWVIQEENKQGSLAPLFEELLQQCPPGGQNKTTHMVSAYQLAQGNWMPVGCHVFMGTIPARRTKTHPYEAYVKLRELVDEYKMKTLCGGSGLSKHNEWVIRKIKHQGNLRTKHMLNPGKVAEQLLREGHRHNVYNKTIGSVMTATGIRLEKLPVVRAQTDTTNFHQAIRDKIDKEENLQTPGLHKKLMEVFNALKRPDLEASYDAVEWEELEKGINRKGAAGFFEHKNIGEVLDSEKNKVEEIIDSLRKGRSIRYYETAIPKNEKRDVNDDWTAGDFVDEKKPRVIQYPEAKTRLAITKVMYKWVKQKPVVIPGYEGKTPLFQIFDKVKKEWDQFQNPVAVSFDTKAWDTQVTTGDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFSSKGVQILYEAGKPQKITEGDKMKVAYQFDDIEFCSHTPVQVRWSDNTSSYMPGRNTTTILAKMATRLDSSGERGTIAYEKAVAFSFLLMYSWNPLIRRICLLVLSTELQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELILARKPISNFEGTDRYNLGPIVNVVLRRLRVMMMALIGRGV

SEQ ID NO: 23 - protein, 3896 aa, Artificial Sequence
  polyprotein of GD18-ddErnsHC-KARD
MELNHFELLYKTNKQKPIGVEEPVYDITGRPFFGDPSEVHPQSTLKLPHDRGRGNIRTTLKNLPRKGDCRSGNHLGPVSGIYVKPGPVFYQDYMGPVYHRAPLEFFEEAQLCEVTKRIGRVTGSDGKLYHIYVCIDGCILLKLAKRGTPRSLKWTRNFTDCPLWVTSCSDDGAGGSKDKKPDRMNKGKLKIAPKEHEKDSKTKPPDATIVVEGVKYQVKKKGKVKGKNTQDGLYHNKNKPPESRKKLEKALLAWAVITIVLYQPVAAENITQWNLSDNGTSGIQQAMYLRGVNRSLHGIWPEKICKGVPTHLATDTELTEIRGMMDASERTNYTCCRLQRHEWNKGWCNWYNIDPWIQLMNRTQANLTEGPPDKECAVTCRYDKNADVNVVTQARNRPTTLTGCKKGKNFSFAGTIIEGPCNFNVSVEDILYGDHEGSLFQDTALYLLDGMTNTIEKARQGAARVTSWLGRQLRTTGKKLERGSKTWFGAYALSPYCNVTRKVGYIWYTNNCTPACLPKNTKIIGPGKFDTNAEDGKILHEMGGHLSEFLLLSLVILSDFAPETASTLYLILHYAIPQSHEEPEGCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLTCKEDYRYAISKTNEIGPLAARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAGLQLGQGEVVLIGNLITHMDNEVVVYFLLLYLIIRDEPIKKWILLLFHAMTNNPVKTMTVALLMISGVAKGGKIDGGWQRQPETNFDIQFALAVIIVVVMLLAKRDPTTFPLVITVATLRTAKITSGFSTDLAIATVSATLLTWTYISDYYKYKTWLQYLISTVTGIFLIRVLKGVGELDLHAPTLPSHRPLFYILVYLISTAVVTRWNLDVAGILLQCAPTLLMVFTMWADILTLILILPTYELTKLYYLKEVKIGTERGWLWKTNYKRVNDIYEVDQAGEGVYLFPSKQKTGAITSTVLPLIKAILISCISNKWQFIYLLYLIFEVTYYLHKKIIDEIAGGTNFVSRLVAALIEVNWALDNEEVKGLKKFFLLSSRVKELVIKHKVRNEVMVRWFEDEEIYGMPKLIGLVKAATLSKNKHCILCTVCEDRDWRGETCPKCGRFGPPVICGMTLADFEEKHYKRIFIREDQSDGPLREEHAGYLQYKARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMEEGYMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDNYTFLNARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAVANYASEGIQFIKSQALKVRETPTYKETMNTVADYVKKFIEALSDSKEDILKYGLWGVHTALYKSIGARLGHETAFATLVVKWLAFGGESVTDHIKQAATDLVVYYIINRPQFPGDTETQQEGRKFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDDIKSSVREIAISWAPAPFSCDWTPTDDRIGLPHDNYLQMETRCPCGYRMKAVKTCAGELRLLEEGGSFLCRNKFGRGSRNYRVTKYYDDNLSEIKPVIRMEGHVELYHKGATFKLDFDNSKTVLATDRWEVDHSTLIRVLKRHTGAGFQGAYLGEKPNHKHLIERDCATITKDKVCFIKMKRGCAFTYDLSLHNLTRLIELVHKNNLDDREIPAVTVTTWLAYTFVNEDIGTIKPVFGEKVTPEKQEEVALQPAVVVDTTDVAVTVVGETSTMTTGETPTIFTSLGSDSKFQQVLKLGVDEGQYPGPRQQRASLLEAVQGVDERPSVLILGSDKATSNRVKTAKNVKIFRSRDPLELREMMRRGKILIIALCKVDTALLKFVDYKGTFLTRETLEALSLGKPKKRDITKTEARWLLCLEGQLEELPDWFAAKEPIFLEANIKRDKYHLVGDIATIKEKAKQLGATDSTKISKEVGAKVYSMKLSNWVIQEENKQGSLTPLFEELLQQCPPGGQNKTTHMASAYQLAQGNWMPVGCHVFMGTIPARRTKTHPYEAYVKLRELVEEHKMKTSCGGSGLCKHNEWVIRKIKHQGNLRTRHMLNPGKIAEQLDREGHRHNVYNKTIGSVMTATGIRLEKLPVVRAQTDTTNFHQAIRDKIDKEENLQTPGLHKKLMEVFNALKRPELEASYDAVEWEELERGINRKGAAGFFERKNIGEVLDSEKYKVEEIIDSLKKGRSIKYYETAIPKNEKRDVNDDWTAGDFVDEKKPRVIQYPEAKTRLAITKVMYKWVKQKPVVIPGYEGKTPLFQIFDKVKKEWNQFQNPVAVSFDTKAWDTQVTTRDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFASRGVQILYEAGKPQKITEGDNMKVAYQFDDIEFCSHTPVQVRWSDNTSSYMPGRNTTTILAKMATRLDSSGERGTIAYEKAVAFSFLLMYSWNPLIRRICLLVLSTEMQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELVLARKPTSNFEGTDRYNLGPIVNVVLRRLRVMMMALIGRGV

SEQ ID NO: 24 - protein, 3733 aa, Artificial Sequence
  polyprotein of GD18-ddNproErnsH-KARD
MELNSDDGAGGSKDKKPDRMNKGKLKIAPKEHEKDSKTKPPDATIVVEGVKYQVKKKGKVKGKNTQDGLYHNKNKPPESRKKLEKALLAWAVITIVLYQPVAAENITQWNLSDNGTSGIQQAMYLRGVNRSLHGIWPEKICKGVPTHLATDTELTEIRGMMDASERTNYTCCRLQRHEWNKGWCNWYNIDPWIQLMNRTQANLTEGPPDKECAVTCRYDKNADVNVVTQARNRPTTLTGCKKGKNFSFAGTIIEGPCNFNVSVEDILYGDHECGSLFQDTALYLLDGMTNTIEKARQGAARVTSWLGRQLRTTGKKLERGSKTWFGAYALSPYCNVTRKVGYIWYTNNCTPACLPKNTKIIGPGKFDTNAEDGKILHEMGGHLSEFLLLSLVILSDFAPETASTLYLILHYAIPQSHEEPEGCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLTCKEDYRYAISKTNEIGPLAARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAGLQLGQGEVVLIGNLITHMDNEVVVYFLLLYLIIRDEPIKKWILLLFHAMTNNPVKTMTVALLMISGVAKGGKIDGGWQRQPETNFDIQFALAVIIVVVMLLAKRDPTTFPLVITVATLRTAKITSGFSTDLAIATVSATLLTWTYISDYYKYKTWLQYLISTVTGIFLIRVLKGVGELDLHAPTLPSHRPLFYILVYLISTAVVTRWNLDVAGILLQCAPTLLMVFTMWADILTLILILPTYELTKLYYLKEVKIGTERGWLWKTNYKRVNDIYEVDQAGEGVYLFPSKQKTGAITSTVLPLIKAILISCISNKWQFIYLLYLIFEVSYYLHKKIIDEIAGGTNFVSRLVAALIEVNWALDNEEVKGLKKFFLLSSRVKELVIKHKVRNEVMVRWFEDEEIYGMPKLIGLVKAATLSKNKHCILCTVCEDRDWRGETCPKCGRFGPPVICGMTLADFEEKHYKRIFIREDQSDGPLREEHAGYLQYKARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMEEGYMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDNYTFLNARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAVANYASEGIQFIKSQALKVRETPTYKETMNTVADYVKKFIEALSDSKEDILKYGLWGVHTALYKSIGARLGHETAFATLVVKWLAFGGESVTDHIKQAATDLVVYYIINRPQFPGDTETQQEGRKFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDDIKSSVREIAISWAPAPFSCDWTPTDDRIGLPHDNYLQMETRCPCGYRMKAVKTCAGELRLLEEGGSFLCRNKFGRGSRNYRVTKYYDDNLSEIKPVIRMEGHVELYHKGATFKLDFDNSKTVLATDRWEVDHSTLIRVLKRHTGAGFQGAYLGEKPNHKHLIERDCATITKDKVCFIKMKRGCAFTYDLSLHNLTRLIELVHKNNLDDREIPAVTVTTWLAYTFVNEDIGTIKPVFGEKVTPEKQEEVALQPAVVVDTTDVAVTVVGETSTMTTGETPTIFTSLGSDSKFQQVLKLGVDEGQYPGPRQQRASLLEAVQGVDERPSVLILGSDKATSNRVKTAKNVKIFRSRDPLELREMMRRGKILIIALCKVDTALLKFVDYKGTFLTRETLEALSLGKPKKRDITKTEARWLLCLEGQLEELPDWFAAKEPIFLEANIKRDKYHLVGDIATIKEKAKQLGATDSTKISKEVGAKVYSMKLSNWVIQEENKQGSLTPLFEELLQQCPPGGQNKTTHMASAYQLAQGNWMPVGCHVFMGTIPARRTKTHPYEAYVKLRELVEEHKMKTSCGGSGLCKHNEWVIRKIKHQGNLRTRHMLNPGKIAEQLDREGHRHNVYNKTIGSVMTATGIRLEKLPVVRAQTDTTNFHQAIRDKIDKEENLQTPGLHKKLMEVFNALKRPELEASYDAVEWEELERGINRKGAAGFFERKNIGEVLDSEKYKVEEIIDSLKKGRSIKYYETAIPKNEKRDVNDDWTAGDFVDEKKPRVIQYPEAKTRLAITKVMYKWVKQKPVVIPGYEGKTPLFQIFDKVKKEWNQFQNPVAVSFDTKAWDTQVTTRDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFASRGVQILYEAGKPQKITEGDNMKVAYQFDDIEFCSHTPVQVRWSDNTSSYMPGRNTTTILAKMATRLDSSGERGTIAYEKAVAFSFLLMYSWNPLIRRICLLVLSTEMQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELVLARKPTSNFEGTDRYNLGPIVNVVLRRLRVMMMALIGRGV

SEQ ID NO: 25 - protein, 5 aa, Artificial Sequence
  VH CDR1
SFGMH

SEQ ID NO: 26 - protein, 17 aa, Artificial Sequence
  VH CDR2
YISSDSFTIYYADTMKG

SEQ ID NO: 27 - protein, 7 aa, Artificial Sequence
  VH CDR3
GDLPFAY

SEQ ID NO: 28 - protein, 11 aa, Artificial Sequence
  VL CDR1
KASQAVGTAVA

SEQ ID NO: 29 - protein, 7 aa, Artificial Sequence
  VL CDR2
WASTRHT

SEQ ID NO: 30 - protein, 9 aa, Artificial Sequence
  VL CDR3
HQYSSYPLT

SEQ ID NO: 31 - protein, 12 aa, Artificial Sequence
  mutated 6B8 epitope with RD for GD18 or QZ07 or GD191
STNEIGPLGARD

SEQ ID NO: 32 - protein, 12 aa, Artificial Sequence
  mutated 6B8 epitope with RD for C-strain
STDEIGLLGARD

SEQ ID NO: 33 - protein, 12 aa, Artificial Sequence
  mutated 6B8 epitope with KRD for C-strain
KTDEIGLLGARD

SEQ ID NO: 34 - protein, 12 aa, Artificial Sequence
  mutated 6B8 epitope with KARD for C-strain
KTDEIGLLAARD

SEQ ID NO: 35 - protein, 373 aa, Artificial Sequence
  C strain-E2
RLACKEDYRYAISSTDEIGLLGAGGLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG